(19) United States
(12) Patent Application Publication
(10) Pub. No.: US 2024/0125803 A1
(43) Pub. Date: Apr. 18, 2024

(54) NOVEL DIAGNOSTIC AND METHOD OF SCREENING THERAPEUTICS FOR FIBROTIC CONDITIONS

(71) Applicant: BLR-Bio, LLC, Kenosha, WI (US)

(72) Inventor: Bruce L. Riser, Kenosha, WI (US)

(21) Appl. No.: 18/466,680

(22) Filed: Sep. 13, 2023

Related U.S. Application Data
(60) Provisional application No. 63/375,445, filed on Sep. 13, 2022.

(51) Int. Cl. G01N 33/68; C12Q 1/6883

(52) U.S. Cl. CPC G01N 33/6893; C12Q 1/6883; C12Q 2600/112; C12Q 2600/136; C12Q 2600/158; G01N 2500/10; G01N 2800/20; G01N 2800/56

(57) ABSTRACT

BLR-200 prevented and treated fibrosis in bleomycin-induced SSc fibrosis, as indicated by impairment of skin thickening, collagen deposition, and myofibroblast activation. BLR-200 treatment prevented bleomycin-induced CCN1 and CCN2 expression. Through single-cell RNA-sequencing analysis, and spatial gene analysis of tissue, specific populations of myofibroblasts could be identified that were responsible for driving the disease initiation and progression not previously identified. BLR-200 impaired the ability of collagen-expressing fibroblasts to respond to bleomycin-induced inflammatory-driven fibrosis, including the creation and expansion of critical sub populations. BLR-200 prevented overexpression of pro-inflammatory genes including Il6, Cxcl2, and NLRP3 inflammasome markers and activation of epithelial cell markers and the Wnt pathway in these populations. CCN proteins play an important role in dermal fibrosis, and other forms of fibrosis including cancer. Targeting the pro-fibrotic activity of CCN1 and CCN2 using endogenously derived CCN3-based peptides, and the creation of myofibroblasts, can prevent multiple pro-fibrotic changes and represents a novel therapeutic approach for treatment of SSc fibrosis. This invention provides for the novel treatment and diagnosis of other inflammatory and fibrosis-related diseases including cancer. And a new method for creating and screening drugs and new diagnostic kits for a variety of diseases is disclosed herein.

### CROSS-REFERENCE TO RELATED APPLICATIONS

The present application claims priority to U.S. provisional application Ser. No. 63/375,445, filed Sep. 13, 2022, herein incorporated by reference in its entirety.

### SEQUENCE LISTING

The present application contains a Sequence Listing which is submitted electronically in XML format and is incorporated by reference in its entirety. Said XML file was created on Jan. 8, 2024, is named XML Sequence BLR-101-US.xml and 18,533 bytes in size.

### BACKGROUND

The invention generally relates to the treatment, diagnosis, and/or prevention of fibrotic and inflammatory diseases and diagnostic systems for the same.

Fibrotic conditions, including those associated with significant inflammation, such as the often-fatal disease systemic sclerosis (SSc), are characterized by a loss of extracellular matrix (ECM) homeostasis and dysregulation of the normal wound healing process, leading to the excessive deposition of ECM components, such as collagen and fibronectin, resulting in pathological scarring that can result in organ failure and death (Leask, 2015; Schulz et al., 2018). In patients with SSc, a number of tissues are affected by fibrosis. In its diffuse form, patients may develop excessive fibrosis in their skin, liver, kidney, oral cavity, and lungs. Symptoms can range in severity and often include ulcers, tight and itchy skin, loss of mobility, and pulmonary hypertension (Allanore et al., 2015; Belloli et al., 2008; Czirják et al., 2008; Lóránd et al., 2014). Currently, there is no approved treatment available for SSc patients, and diagnosis or determination of disease stage, prediction of remission or progression is unavailable. This issue is due, in great part, to the lack of suitable, specific targets, as well as a lack of unbiased, reliable, non-invasive methods for tracking disease progression (Varga & Abraham, 2007). These observations highlight the need to characterize the disease, as well as determine a suitable targets on which to base therapeutic intervention.

In fibrotic lesions, resident fibroblasts within the ECM become activated and differentiate into myofibroblasts (Hinz, 2010). A critical indicator that distinguishes clinically involved from clinically uninvolved tissue in SSc patients is the presence of myofibroblasts (Rajkumar et al., 2005). Recent evidence suggests that the myofibroblast population in fibrotic lesions are derived, at least in part, from collagen-lineage resident dermal fibroblasts that become activated through a progenitor cell-like intermediate (Liu et al., 2014; Tsang et al., 2019). These myofibroblasts intrinsically possess abnormally active adhesive signalling and will excessively synthesize, adhere to and contract ECM. Activated myofibroblasts are characterized by excessive collagen secretion and overexpression of the highly contractile protein α-smooth muscle actin (αSMA) which contributes to the increased mechanical tension of fibrotic tissue (Hinz, 2015). Increased mechanical tension in the ECM can lead to further activation of resident fibroblasts into active myofibroblasts (Hinz, 2019). Thus, fibrosis is maintained by an autocrine, pro-adhesive signaling loop. Broad targeting of this pathway may have deleterious effects on normal physiology. Thus, targeting specific downstream mediators of this feedback loop may represent a viable anti-fibrotic therapy.

A growing body of evidence has shown that the Cellular Communication Network (CCN) family of matricellular proteins is an important contributor to myofibroblast activation, and sustained adhesive and contractile signalling in fibrosis (Leask, 2013; Leask & Abraham, 2006; Perbal, 2004). The CCN family comprises six secreted proteins containing highly similar modular structure. Although originally thought to act as traditional growth factors, it is now known that the CCN proteins primarily function by modulating the signaling of other ECM molecules, such as transforming growth factor β (TGFβ) (Leask & Abraham, 2004). CCN proteins are known to play a role in development, inflammation, tissue repair, and a broad range of pathological processes including fibrosis and cancer (Chen & Lau, 2009; Perbal, 2013). The activity of CCN proteins is complex and highly context-dependent, with varying responses depending on the biological system. To date it has not been shown exactly how CCNs work. Thus, it is essential to study CCN protein function in vivo.

The most well studied member of the CCN family, CCN2 (formerly CTGF) is induced in differentiated myofibroblasts during wound healing and is overexpressed in fibrotic disease, as illustrated by the presence of CCN2 in the blister fluid of SSc patients (Holmes et al., 2011; Leask, 2013). CCN2 is a key, selective downstream mediator of fibrosis in multiple fibrotic models, including cardiac, renal and hepatic fibrosis (Dorn et al., 2018; Parapuram et al., 2015; Phanish et al., 2010; Rachfal & Brigstock, 2003). In fact, CCN2 expression by fibroblasts is required for fibrosis development in bleomycin-induced dermal fibrosis (Liu et al., 2011). Furthermore, CCN2 is required for recruitment and activation of collagen-lineage resident dermal fibroblasts into αSMA-expressing myofibroblasts (Liu et al., 2014, Tsang et al., 2019).

Another member of the CCN family, CCN1 (formerly CYR61), has structural and functional similarities to CCN2 in vitro and in vivo (Jun & Lau, 2011; Perbal, 2018). Although the role of CCN1 in pathological processes such as fibrosis is less known, CCN1 has been shown to play a context-dependent role in promoting inflammatory responses, including in SSc (Kubota & Takigawa, 2007; Quesnel, 2019). CCN1 is also induced by pro-fibrotic stimuli in vitro (Thompson et al., 2014). Like CCN2, CCN1 expression by fibroblasts is required for bleomycin-induced dermal fibrosis, and likely plays a crucial role in collagen deposition and accumulation in the dermis (Quesnel et al., 2019). Altogether, there is ample evidence that CCN1 and CCN2 represent viable anti-fibrotic targets.

Recent work has shown that another member of the CCN family, CCN3, is reciprocally regulated to CCN1 and CCN2 in several cell types, and also exhibits opposing functional behaviour (Abd El Kader et al., 2012; Lemaire et al., 2010; Perbal, 2018; Riser et al., 2009). Furthermore, treatment with recombinant human CCN3 (rhCCN3) was able to block and reverse fibrosis in a mouse model of diabetic nephropathy (Riser et al., 2014). Taken together, these results suggest that CCN3 may act as an endogenous antagonist of CCN1 and CCN2 activity. These discoveries led to the development of a proprietary CCN3-based peptide (BLR-200) based on regions of CCN3 responsible for its anti-fibrotic effects in vivo. Initial studies have shown that BLR-200 can block mesangial cell adhesion to CCN2 and inhibit the ability of pro-fibrotic agents to provoke collagen expression in dermal fibroblasts in vitro (U.S. Pat. No. 9,114,112B2, 2015).

Fibrotic conditions are characterized by the excessive deposition of extracellular matrix (ECM) components, often due to dysregulation of the normal wound healing process, leading to pathological scarring that can result in organ failure and, ultimately, death (Schulz et al., 2018; Walraven & Hinz, 2018). An example of such a fibrotic condition is the autoimmune connective tissue disorder systemic sclerosis (SSc). Patients with SSc often experience progressive fibrosis of the skin and internal organs, including the lung, heart, gastrointestinal tract, and kidneys (Leask, 2015). The mortality rates associated with SSc are frequently high, and patients will often die from lung fibrosis (Denton et al., 2018). There is currently no approved therapy for SSc, in part due to its unknown etiology, highlighting the need to further characterize the mechanism underlying disease. Since SSc shares similar features with other fibrotic disorders, understanding the molecular basis of SSc may reveal pertinent information regarding pathological fibrosis in general. This understanding these pathways and mechanisms can provide a platform for discovery and screening new drugs and for determining patients more likely to respond to therapy in clinical trials and beyond with eventual approved drugs. It can provide information on the critical markers, or combination of markers to stage disease as well as to determine a early response to therapy to optimize drug use and dose.

In SSc patients, a notable early pathological change in clinically involved connective tissue is vascular injury, which leads to infiltration of immune mediators, such as activated T-cells and macrophages (Allanore et al., 2015; Cutolo et al., 2019). Infiltration of these immune cells results in the release of several pro-inflammatory and pro-fibrotic cytokines, including interleukin-1 (IL-1), IL-6, and transforming growth factor β (TGFβ) isoforms (King et al., 2018; Muangchan & Pope, 2012). These cytokines promote a pro-fibrotic microenvironment which activates keratinocytes and fibroblasts, leading to fibrosis (King et al., 2018; Russo et al., 2021).

The critical responder cell type responsible for fibrogenesis, including in SSc, is a particular type of activated fibroblast, termed the myofibroblast (Chen et al., 2005; Gabbiani, 2003; Hinz, 2010, 2015). One of the defining features of activated myofibroblasts is the expression of the highly contractile protein α-smooth muscle actin (αSMA) (Gabbiani, 2003). Once activated, myofibroblasts will excessively synthesize ECM components such as type I collagen and fibronectin, as well as adhere to and contract the surrounding ECM (Hinz, 2015). Although myofibroblast differentiation is required for normal wound repair, in this physiological process myofibroblasts disappear, due to apoptosis, after wound closure (Desmouliere et al., 1995). Conversely, in pathological fibrosis, myofibroblasts persist. As immune infiltration causes fibroblasts to differentiate into myofibroblasts, studying how fibroblasts respond to the inflammatory microenvironment provide valuable information to our overall understanding of fibrogenesis.

The Cellular Communication Network (CCN) family of matricellular proteins is the subject of many recent studies, as their collective action has been shown to contribute to fibrogenesis (Leask, 2020b). In general, CCN proteins play a role in modulating cellular responses to the local microenvironment, it appears, by directly binding to adhesion receptors, such as integrins, as well as by acting as co-factors for other regulatory molecules (Lau, 2016; Leask, 2020b). The wide array of actions mediated by CCN proteins depends on the presence of the proteins and receptors with which they interact (Leask, 2020b). Therefore, their contribution to pathological conditions, such as fibrosis, is extremely context- and stage-specific, specifically integrating signals that are occurring in a specific location, at a specific point in time.

CCN2 is induced in differentiated myofibroblasts during wound healing and is accepted as a marker of myofibroblasts in pathological fibrosis (Perbal, 2018). Furthermore, CCN2 expression by fibroblasts is required for fibrosis in multiple mouse models of SSc (Liu et al., 2011, 2013). CCN1 expression by fibroblasts is also required for dermal fibrosis in mouse models, and likely plays an important role in collagen organization (Quesnel et al., 2019; Shi-wen et al., 2021). Of clinical importance, both CCN1 and CCN2 expression are increased in the fibrotic lesions of early onset diffuse SSc patients, providing evidence that these proteins may play crucial roles in SSc fibrosis (Holmes et al., 2011). Furthermore, CCN1 and CCN2 deletion from fibroblasts had no significant effects on the normal wound healing process (Liu et al., 2014; Quesnel et al., 2019). Considering these specific effects of CCN1 and CCN2 on fibrosis, targeting these proteins may represent a potential anti-fibrotic approach.

Another member of the CCN family, CCN3, is reciprocally regulated to CCN1 and CCN2 in several cell types, often exhibiting opposing functional behavior; thus, CCN3 may act as an endogenous antagonist of the pro-fibrotic activity of CCN1 and CCN2 (Abd El Kader et al., 2012; Lemaire et al., 2010; Perbal, 2018; Riser et al., 2015). For example, treatment with recombinant human CCN3 blocks and reverses fibrosis in a mouse model of diabetic nephropathy (Riser et al., 2009, 2014). These observations culminated in the development of a proprietary CCN3-derived peptide (BLR-200) which mimicked the in vivo anti-fibrotic ability of intact, full-length CCN3. According to an issued patent, BLR-200 blocks mesangial cell adhesion to CCN2 and inhibits the ability of pro-fibrotic agents to provoke collagen expression in dermal fibroblasts in vitro (U.S. Pat. No. 9,114,112B2, 2015). Therefore, BLR-200 may represent a novel therapeutic option for SSc.

BLR-200 treatment prevents progressive dermal fibrosis in the bleomycin-induced model of SSc. This model mimics the initial stages of SSc dermal fibrosis, histologically and biochemically, and thus is useful to study fibrogenesis (Yamamoto, 2017; Yamamoto et al., 1999). Repeated subcutaneous injection of bleomycin causes a short-lived inflammatory response that leads to progressive fibrosis after 3-4 weeks (Yamamoto, 2006). BLR-200 prevents fibrotic changes after 28 days. However, the effect of BLR-200 on the response of fibroblasts to inflammation, prior to the development of fibrosis, remains to be determined.

Previous studies employing standard analytical techniques aimed at understanding the central mechanisms that drive pathological fibrosis have shown limited progress. These limitations are largely due to the complexity and heterogeneity of tissue changes that occur during fibrogenesis. However, the use of unbiased single-cell RNA-sequencing (scRNA-seq) may offer the potential to overcome such limitations. Recent studies have shown that scRNA-seq analysis of fibroblast heterogeneity in fibrosis provides useful information on the diversity of aberrant cell populations present in fibrotic lesions (Deng et al., 2021; Vorstandlechner et al., 2020). Furthermore, unbiased transcriptomic analysis could further our understanding of key mechanistic mediators of fibrosis.

The present invention attempts to solve these problems, as well as others.

### SUMMARY OF THE INVENTION

Provided herein are systems, methods and compositions for preventing and treating inflammatory and fibrotic diseases and diagnostic systems for diagnosing, staging, and determining the response to therapy. BLR-200 prevents, blocks, and reverses progression of inflammatory-induced skin fibrosis, and reduces collagen deposition, myofibroblast formation, dermal progenitor cell activation and YAP1 expression, impaired response to inflammation in inflammatory subset of collagen-lineage fibroblasts, inflammasome signature collagen-lineage fibroblasts reduced, impaired induction of egr1 in synthetic subset of collagen-lineage fibroblasts, and mechanism of BLR-200 action involves several pathways known to be essential in SSc fibrogenesis

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIGS. 1A-1F are representative microscope images of skin stained for collagen deposition. FIGS. 1A and 1B show the skin of the normal healthy mice group (PBS). FIGS. 1C and 1D receiving bleomycin show marked thickening of the skin. The mouse group receiving bleomycin plus BLR-200 treatment (FIGS. 1E and 1F) are indistinguishable from the healthy control group (PBS). Skin thickness was determined by measuring the dermal layer (red arrow). Collagen deposition was measured by ImageJ. Masson's trichrome image was deconvolved using color deconvolution plugin in ImageJ. Green channel image represented the collagen fibers. FIGS. 1G-1H are bar graphs showing data is a quantitation of the measurement of the green channel wavelength in multiple samples for each of 5 animals per group and presented as mean±SEM, n=5. Statistical analysis was performed using one-way ANOVA followed by Tukey-Kramer test. *p≤0.05, **p≤0.01

FIGS. 2A-2I are microscope images of BLR-200 suppressing myofibroblast differentiation in bleomycin-induced fibrosis at day 21. Immunofluorescence was performed using αSMA antibody, a marker of myofibroblast. FIGS. 2A-2C are PBS treated control animals. FIGS. 2D-F are bleomycin treated animals. FIGS. 2G-2I are bleomycin plus BLR-200. FIG. 2J is a graph showing BLR-200 suppressed myofibroblast differentiation in bleomycin-induced fibrosis at d21 and the data was confirmed by quantification of % area of myofibroblasts. Data was presented as mean±SEM, n=5. Statistical analysis was performed using one-way ANOVA followed by Tukey-Kramer test.

FIGS. 3A-3D are graphs showing the changes in key genes in autoimmune/inflammatory SSc modeled skin disease and the positive drug effect of BLR-200 on the mRNA expression of fibrosis related genes determined by RT-PCR. The mRNA expression of mechanical sensing YAP1 (FIG. 3A), extracellular matrix glycoprotein tenasin C (TNC) (FIG. 3B), collagen crosslinking enzymes PLOD2 (FIG. 3C) and multipotential neural crest stem cell marker SOX2 (FIG. 3D). All these elements increased with disease and were blunted by BLR-200. Data was presented as mean±SEM, n=5. Statistical analysis was performed using one-way ANOVA followed by Tukey-Kramer test. *p≤0.05, **p≤0.01.

FIG. 4 is an mRNA visualization of cells (using transcriptomics) in tissues show the changes of key genes in disease, and that BLR-200 suppressed the expression of genes related to fibrosis at d21. Spatial gene expression was performed using 10× Genomics's Visium Spatial Gene Expression on formalin fixed paraffin embedded skin samples.

FIGS. 5A-5F are microscope images of treatment with Bleomycin plus BLR-200 (FIGS. 5E-5F) beginning at d14 post bleomycin injection, bleomycin alone for the entire 28 days (FIGS. 5C-5D), or control PBS given for 21 days (FIGS. 5A-5B). BLR-200 given 3× per week for the remainder of the experiment, significantly reversed excessive collagen deposition at d28. Collagen deposition analysis was performed using color deconvolution method in ImageJ. FIG. 5G is a graph showing the data was presented as mean±SEM, n=4. Statistical analysis was performed using one-way ANOVA followed by Tukey-Kramer test. *p≤0.05,

FIGS. 6A-6C are microscope images showing BLR-200 prevents the bleomycin-induced increase in skin thickness. Bleomycin sulfate plus a control scrambled peptide (FIG. 6B, 0.1 units/100 ml per injection) or PBS (FIG. 6A, 100 ml per injection) was injected subcutaneously into a single location on the flank of wild-type C57BL/6J mice once daily for 28 days. Another bleomycin-treated mice group were injected intraperitoneally 3 times per week with BLR-200 (FIG. 6C, BLR-200). At the conclusion of the experiment, dermal tissue was fixed and stained with H&E. Representative images are shown. FIG. 6D is a graph showing the dermal thickness measurements were taken at 3 different points along 2 depths of skin, averaged for each mouse, and compared among treatment groups (one-way ANOVA with Tukey's post hoc test; *p<0.005). Bleomycin increased skin thickness while BLR-200 returned it to the normal thickness of the healthy control mice.

FIGS. 7A-7C are microscope images of BLR-200 prevents bleomycin-induced collagen changes in the dermis. Wild-type C57BL/6J mice were either treated with PBS (FIG. 7A, n=6), bleomycin+a scrambled control peptide (FIG. 7B, BLM; n=6), or bleomycin+BLR-200 (FIG. 7C, BLR-200; n=6). Dermal tissue was stained with Masson's trichrome stain to examine histological changes in collagen organization. Images were taken at 3 different points along 2 different depths of skin. Representative images are shown. FIG. 7D is a graph showing the percent area of the dermal layer stained for trichrome was determined using ImageJ (one-way ANOVA with Tukey's post hoc test; **p<0.002; *p<0.05). FIGS. 7E-7F are graphs showing the total RNA was obtained from skin and subjected to SYBR Green real-time PCR. Primers were used to detect collagen cross-linking genes Plod2 (FIG. 7E) and Lox (FIG. 7F). Relative gene expression, compared to the housekeeping gene Rn18s, was determined using the ΔΔCt method (one-way ANOVA and Tukey's post-host test; **p<0.002; *p<0.05). FIGS. 7G-7I are microscope images of skin sections were stained with picrosirius red and collagen birefringence properties were analyzed to investigate newly synthesized collagen and fiber density. FIG. 7J is a graph showing the one-way ANOVA and Tukey's post-host test were used to analyze the data (*p<0.05).

FIGS. 8A-8C are microscope images showing BLR-200 impairs bleomycin-induced increase in αSMA-expressing myofibroblasts. Wildtype C57BL/6J mice were either treated with PBS (FIG. 8A, n=6), bleomycin+scrambled peptide (FIG. 8B, BLM; n=6), or bleomycin+BLR-200 (FIG. 8C, BLR-200; n=6). Fixed dermal tissue sections were incubated with an anti-αSMA antibody and antibody localization was visualized using DAB chromogen. Representative images are shown. FIG. 8D is a graph showing the percentage of fibroblasts positive for αSMA was determined using ImageJ (one-way ANOVA and Tukey's post hoc test; **p<0.002; *p<0.05).

FIG. 8E is a graph showing the total RNA was extracted from the skin and subjected to SYBR Green real-time PCR using primers detecting Acta2 (murine gene encoding αSMA). Relative Acta2 expression, compared to the housekeeping gene Rn18s, was determined using the ΔΔCt method (one-way ANOVA and Tukey's post-hoc test; *p<0.05).

FIGS. 9A-9B are graphs showing the BLR-200 prevents changes in protein expression patterns associated with bleomycin-induced dermal fibrosis. Wildtype C57BL/6J mice were either treated with PBS (n=6), bleomycin+scrambled peptide (BLM; n=6), or bleomycin+BLR-200 (BLR-200; n=6). Total protein was extracted from the skin and subjected to tandem mass-tag mass spectrometry. Relative protein expression for BLM mice and BLR-200 mice was normalized to the control PBS group, generating fold change differences for all detected proteins. A list of upregulated proteins in each experimental group was generated using a 1.8-fold cut-off (p<0.05). (FIGS. 9A-9B) Venn diagram and table showing that 124 proteins that were induced in BLM mice are prevented by BLR-200 treatment. (FIG. 9C) Reactome pathway analysis of bleomycin-induced proteins that were prevented by BLR-200 treatment shows that the many of the top pathways prevented include ECM-associated processes, including keratinization, extracellular matrix organization, collagen formation, and laminin interactions. FIGS. 9D-9J are graphs examining fold change differences of individual proteins revealed that specific laminins (a3, a5, b3, c2) and fibrosis-related proteins (nectin-2, pecam-1, fibromodulin) were prevented by BLR-200 treatment. A student's t-test was used to analyze differences in fold change (*p<0.05).

FIGS. 10A-10T are microscopic images and graphs showing that BLR-200 treatment prevents bleomycin-induced changes in CCN1 and CCN2. Wildtype C57BL/6J mice were either treated with PBS (FIGS. 10A-10C, 10K-10M, 10U-10W, n=6), bleomycin+scrambled peptide (FIGS. 10D-10F, 10N-10P, 10X-10Z, BLM; n=6), or bleomycin+BLR-200 (FIGS. 10G-10I, 10Q-10S, 10AA-10CC, BLR-200; n=6). Dermal tissue was fixed, stained and visualized by immunohistochemistry with primary antibodies against CCN1, CCN2, and CCN3. Representative images are shown. Total RNA was also extracted from the skin and subjected to SYBR Green real-time PCR using primers detecting Ccn1, Ccn2 and Ccn3. FIGS. 10J, 10T, 10DD are graphs showing the relative mRNA expression, compared to the housekeeping gene Rn18s, was determined using the ΔΔCt method (one-way ANOVA and Tukey's post-hoc test; *p<0.05). BLR-200 prevents bleomycin-induced increase in (FIGS. 10B, 10E, 10H) CCN1 and (FIGS. 10L, 100, 10R) CCN2 protein and mRNA expression. FIGS. 10V, 10Y, 10B, 10DD show no significant changes were observed with CCN3 expression.

FIGS. 11A-11I are microscope images of BLR-200 treatment prevents expression of the plasticity marker SOX2. Wildtype C57BL/6J mice were either treated with PBS (FIGS. 11A-11C, n=6), bleomycin+scrambled peptide (FIGS. 11D-11F, BLM; n=6), or bleomycin+BLR-200 (FIGS. 11G-11I, BLR-200; n=6). (FIGS. 11A-11I) Dermal tissue was fixed, stained and visualized by immunohistochemistry with primary antibodies against SOX2. Representative images are shown. FIGS. 11J-11K are graphs showing the total RNA was extracted from the skin and subjected to real-time PCR using primers detecting Sox2 and ItgA11. Relative Sox2 and Itga11 expression, compared to the housekeeping gene Rn18s, was determined using the ΔΔCt method (one-way ANOVA and Tukey's post-hoc test; *p<0.05).

FIGS. 12A-12C are microscope images BLR-200 treatment did not significantly affect bleomycin-induced changes in YAP1 expression in the dermal layer under these conditions. Wildtype C57BL/6J mice were either treated with PBS (FIG. 12A, n=6), bleomycin+scrambled peptide (FIG. 12B, BLM; n=6), or bleomycin+BLR-200 (FIG. 12C, BLR-200; n=6). FIGS. 12A-12C show fixed dermal tissue sections were incubated with an anti-YAP1 antibody and antibody localization was visualized using DAB chromogen. Representative images are shown.

FIG. 12D-12E are graphs showing the percentage of fibroblasts positive for YAP1 was determined using ImageJ (one-way ANOVA with Tukey's post hoc test; *p<0.002; *p<0.05). FIGS. 12E-12B are graphs showing the total RNA was extracted from the skin and subjected to SYBR Green real-time PCR using a primer for Yap1. Relative Yap1 expression, compared to the housekeeping gene Rn18s, was determined using the ΔΔCt method (one-way ANOVA and Tukey's post-hoc test; *p<0.05). FIGS. 12F-12L are graphs showing the BLR-200 Blocks Bleomycin-induced mRNA Expression. Mice were subjected, for 21 days, to bleomycin-induced skin fibrosis in the presence or absence of BLR-200 or scrambled peptide. mRNAs were extracted and mRNA expression [cadherin 11 (FIG. 12F, CDH11), Smad3 (FIG. 12G), tenascin-C (FIG. 12H, TNC), YAP1 (FIG. 12I), Sox2 (not shown), WNT4 (FIG. 12J), frizzled 6 (FZD6, FIG. 12K) and PLOD2 (FIG. 1L) was evaluated by real-time polymerase chain (PCR), relative to b-actin control, using the DDCt method.

FIG. 13A-13B are microscope images showing BLR-200 does not have any significant effect on normal healthy tissue. Wild-type C57BL/6J mice were treated with PBS+scrambled peptide (FIG. 13A, n=6) or PBS+BLR-200 (FIG. 13B, n=6). At the conclusion of the experiment, dermal tissue was fixed and stained with H&E. Representative images are shown. FIG. 13E is a graph showing the dermal thickness measurements were taken at 3-6 random points in each section, averaged for each mouse, and compared (students t-test; *p<0.05). FIGS. 13C-13D are microscope images showing the dermal tissue was stained with Masson's trichrome stain to examine histological changes in collagen organization. Images were taken at 3-6 random points in each section. Representative images are shown. FIG. 13F is a graph showing the percent area of the dermal layer stained for trichrome was determined using ImageJ (student's t-test; *p<0.05). FIGS. 13G-13K are graphs showing the total RNA was extracted from the skin and subjected to SYBR Green real-time PCR using primers detecting Ccn1, Ccn2, Ccn3, Lox, and Plod2. Relative mRNA expression, compared to the housekeeping gene Rn18s, was determined using the ΔΔCt method (one-way ANOVA and Tukey's post-hoc test; *p<0.05).

FIGS. 14A-14C are tables showing BLR-200 prevents early fibrotic changes in the proteome of bleomycin-induced dermal fibrosis. Mice were treated with either PBS (n=3), bleomycin+scrambled peptide (BLM; n=3) or bleomycin+BLR-200 (BLR-200; n=3). Total protein was extracted from the skin and subjected to tandem mass-tag mass spectrometry. Relative protein expression for BLM mice and BLR-200 mice was normalized to the control PBS group, generating fold change differences for all detected proteins. A list of upregulated proteins in each experimental group was generated using a 1.8-fold cut-off. FIG. 14A is a Venn diagram showing that 50 out of 106 proteins induced in BLM mice are prevented by BLR-200 treatment. FIG. 14C Reactome pathways analysis of bleomycin-induced proteins that were prevented by BLR-200 treatment shows that the top pathways prevented involve keratinocyte activation and smooth muscle contraction. FIGS. 14D-14E are tables showing the analysis of fold change differences for proteins that were prevented by BLR-200 treatment. Markers of epithelial activation (FIG. 14D, keratin 16, keratin 6, keratin 5, keratin 14, keratin 1, fillagrin, and hornerin), and FIG. 14E shows the markers of early fibrogenesis (fibrillin-1 and dermatopontin) and myofibroblast activation (myosin light chain 6b and tropomyosin 3) were prevented by BLR-200 treatment.

FIG. 15 is a schematic showing the isolation of collagen-lineage fibroblasts from the fibrotic lesion of mice subjected to bleomycin-induced dermal fibrosis. Illustration of workflow for labeling and isolation of collagen-lineage fibroblasts for scRNA-seq analysis. Collagen-lineage fibroblasts were labeled with GFP using a fibroblast specific collagen promoter/enhancer. Briefly, experimental mice were hemizygous for tamoxifen-dependent Cre recombinase under the control of the fibroblast specific proa2 collagen promoter (Col1a2-Cre(ER)-T; Denton et al., 2001) and homozygous for a double fluorescent reporter transgene (mT/mG) integrated into the Gt(ROSA)26Sor locus (GT(ROSA)26mTmG; Muzumdar et al., 2007). At 3 weeks of age, mice were injected with tamoxifen to induce GFP expression. After two weeks, bleomycin sulfate (0.1 units/100 μl per injection) or PBS (100 μl per injection) was injected subcutaneously into a single location on the flank of the mice once daily for 10 days. Bleomycin-treated mice were further divided into two treatment groups, which were injected intraperitoneally 3 times per week with either 10 μg/kg scrambled peptide or 10 μg/kg BLR-200. At the conclusion of the experiment, dermal tissue from the fibrotic lesion was collected. Fibroblasts were extracted from the dermis and GFP-expressing cells were isolated by fluorescence activated cell sorting before being subjected to scRNA-seq analysis.

FIG. 16A-16I are graphs show the identification of collagen-lineage fibroblast subpopulations in bleomycin-induced dermal fibrosis. Mice were treated with either PBS (FIGS. 16D, 16G, n=3), bleomycin+scrambled peptide (FIGS. 16E, 16H, BLM; n=3) or bleomycin+BLR-200 (FIGS. 16F, 16I, BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. FIGS. 16A-16B are graphs of unsupervised UMAP clustering of cells in each treatment group yielding a total of 7 distinct clusters among the different treatments, comprising 2 inflammatory fibroblast clusters (IF1, IF2), 3 reticular-secretory fibroblast clusters (SC1, SC2, SC3), 1 mesenchymal fibroblast cluster (MES), and 1 unknown cluster. FIGS. 16A-16C Violin plots of inflammatory fibroblast marker genes for cell type identification. Inflammatory fibroblast markers include: Cxcl13, Csf2, Il33. FIGS. 16G-16I are violin plots of reticular-secretory fibroblast marker genes for cell type identification. Reticular-secretory fibroblast markers include: Mgp and Sparcl1.

FIGS. 17A-17I are graphs showing BLR-200 prevents the bleomycin-induced transcriptomic changes in the inflammatory fibroblast subpopulation. Mice were treated with either PBS (FIGS. 17C, 17G, n=3), bleomycin+scrambled peptide (FIGS. 17D, 17H, BLM; n=3) or bleomycin+BLR-200 (FIGS. 17D, 17I, BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. FIGS. 17A-17C are unsupervised UMAP clustering of cells reveals that BLM mice have a shift in the inflammatory subpopulation. The inflammatory subpopulation of BLM mice expresses higher levels of the pro-fibrotic, pro-inflammatory Il6. Cells are colored based on Log 2(Il6 expression). FIGS. 17D-17I are violin plots showing Log 2 expression of Il6 and Cxcl2. BLR-200 prevents the bleomycin-induced increase in gene expression of Il6 and Cxcl2 in the inflammatory fibroblast subpopulation.

FIGS. 18A-18O show that BLR-200 prevents expression of an NLRP3 inflammasome-related gene signature. Mice were treated with either PBS (FIGS. 18A, 18D, 18G, 18J, 18M, n=3), bleomycin+scrambled peptide (FIGS. 18B, 18E, 18H, 18K, 18N, BLM; n=3) or bleomycin+BLR-200 (FIGS. 18C, 18F, 18I, 18L, 18O, BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. FIGS. 18A-18C Unsupervised UMAP clustering of cells reveals that expression of NLRP3 inflammasome markers Il18 and Tir2 is lower in BLR-200 treated mice. Cells are colored based on Log 2(Il18 and Tlr2 expression). Violin plots also reveal that BLR-200 impairs expression of inflammasome-related markers (FIGS. 18D-18F) Il18, (FIGS. 18G-18I) Tlr2, (FIGS. 18J-18L) Il1r1, and (FIGS. 18M-18O) Il1r2.

FIGS. 19A-19I show that BLR-200 has specific effects on pro-fibrotic transcription factors. Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. (FIGS. 19A-19C) Violin plot showing Log 2 expression of Egr1. BLR-200 prevents the bleomycin-induced increase in gene expression of Egr-1 in the Secretory-reticular (SC2) subpopulation. Violin plots of Activator protein (AP1) transcription factor complex components, showing Log 2 expression of (FIGS. 19D-19F) Fosb and (FIGS. 19H-19I) Junb.

FIGS. 20A-20R show that BLR-200 prevents the appearance of a mesenchymal fibroblast subpopulations. Mice were treated with either PBS (FIGS. 20A, 20D, 20G, 20J, 20M, 20P, n=3), bleomycin+scrambled peptide (FIGS. 20B, 20E, 20H, 20K, 20N, 20Q, BLM; n=3) or bleomycin+BLR-200 (FIGS. 20C, 20F, 20I, 20L, 20O, 20R, BLR-200; n=3) for 10 days. At the conclusion of the experiment, collagen-lineage fibroblasts were extracted and isolated by FACS.

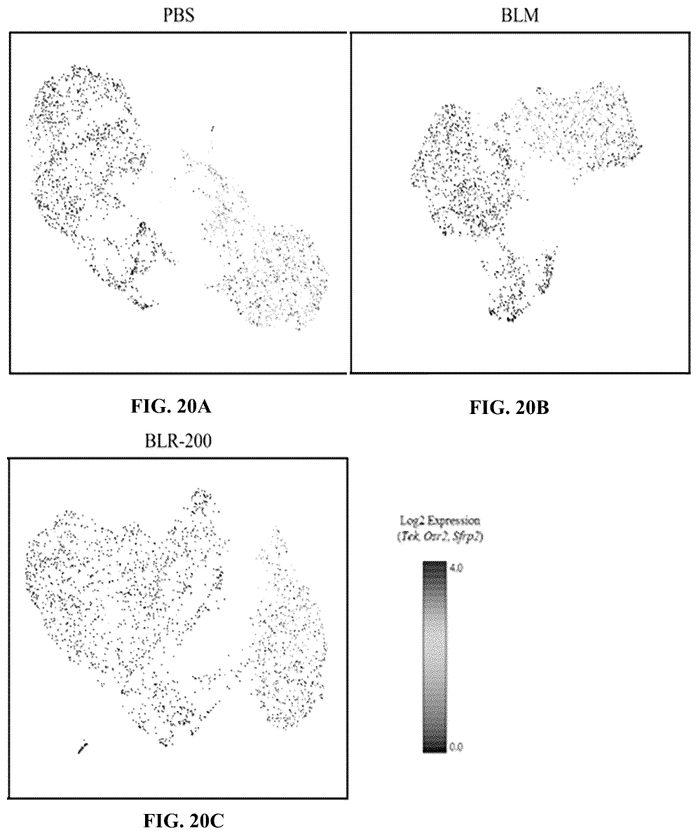

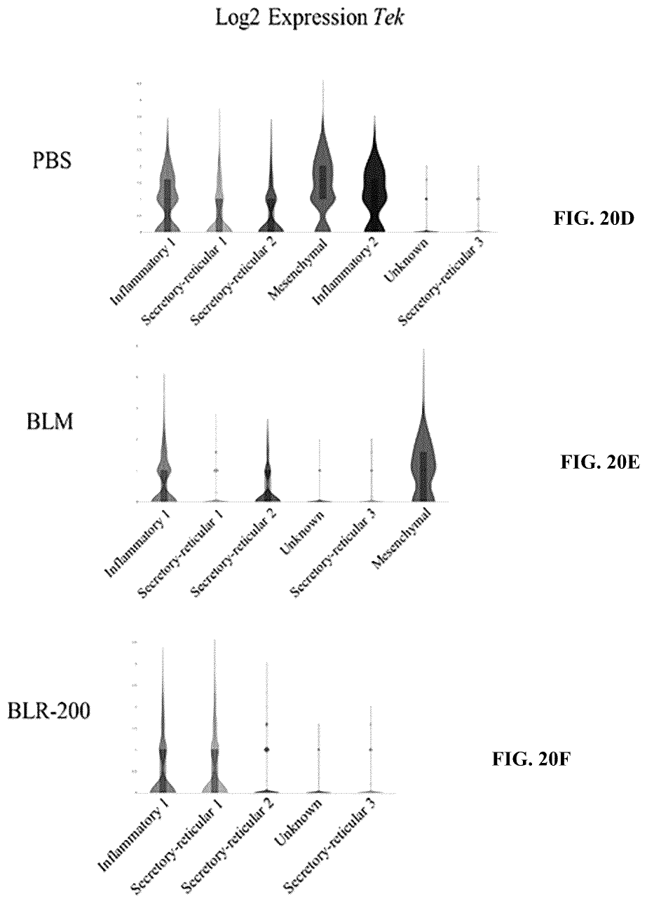

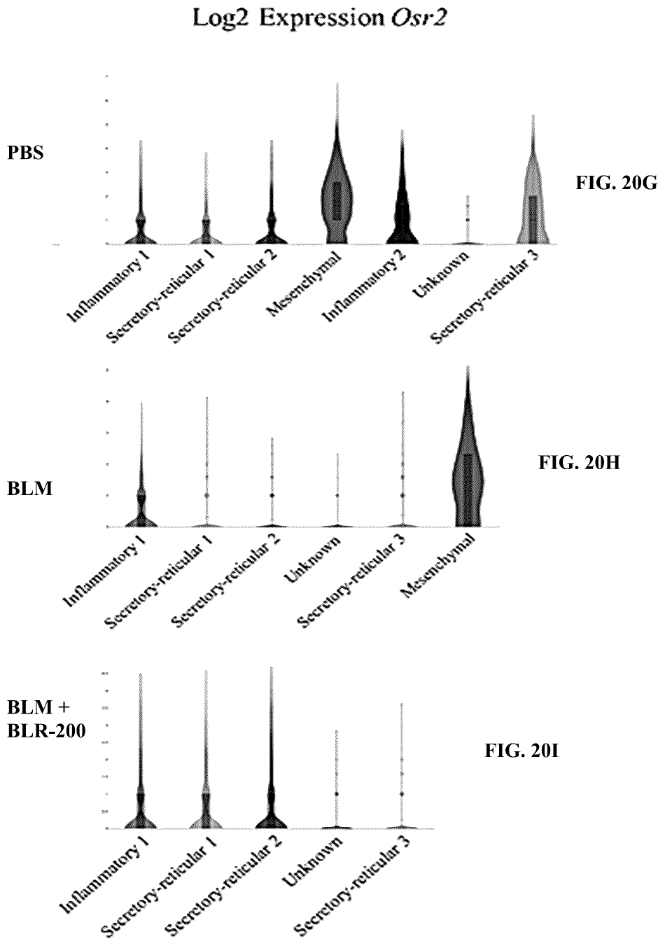

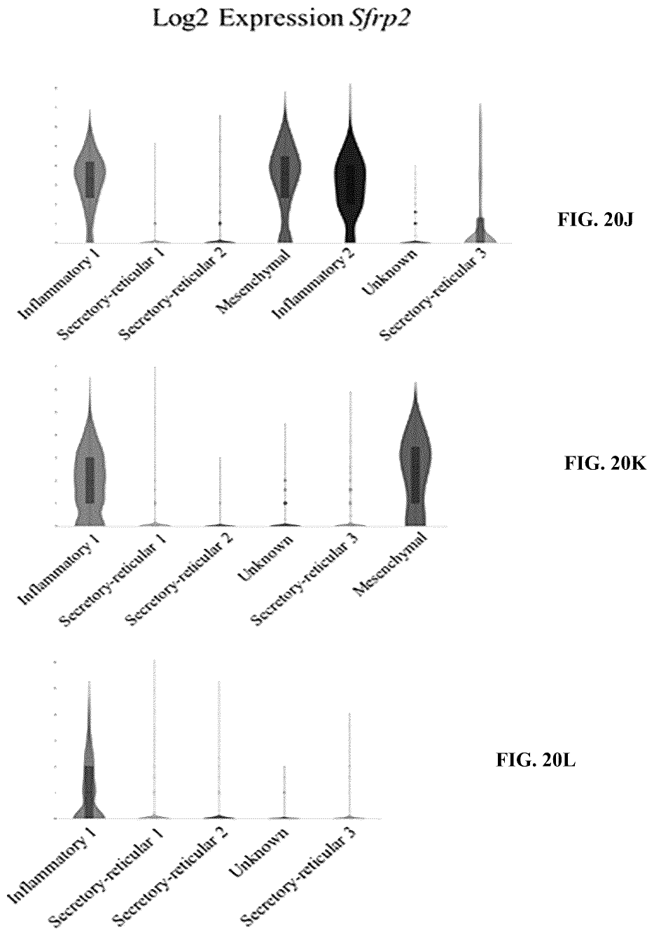

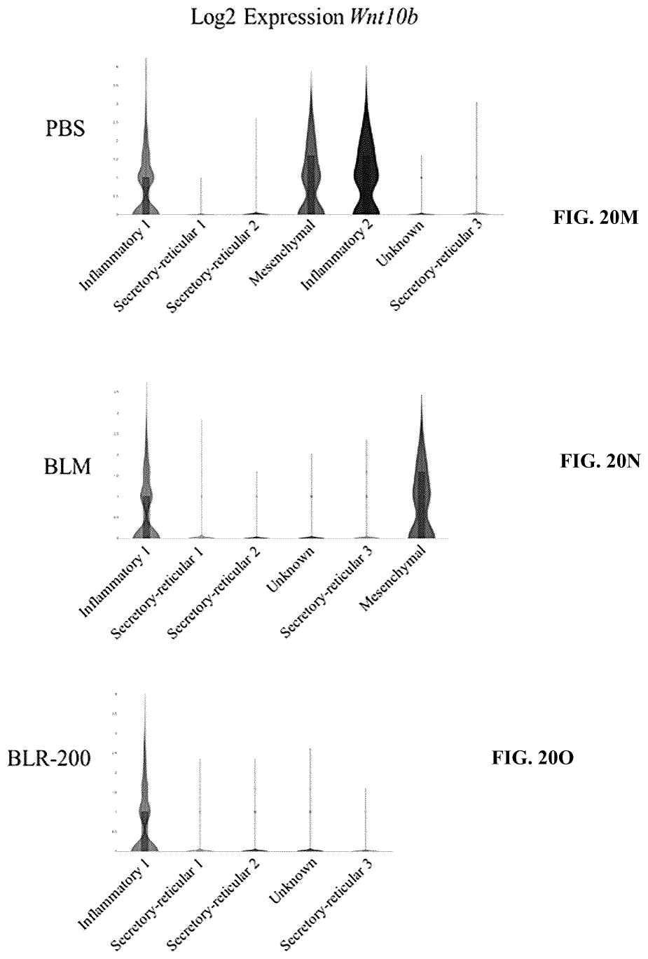

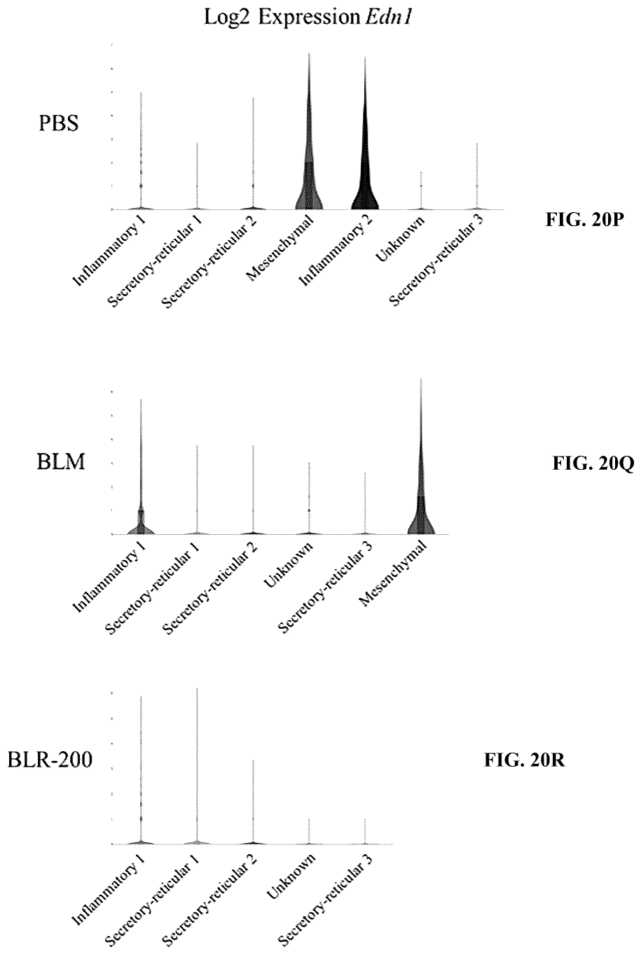

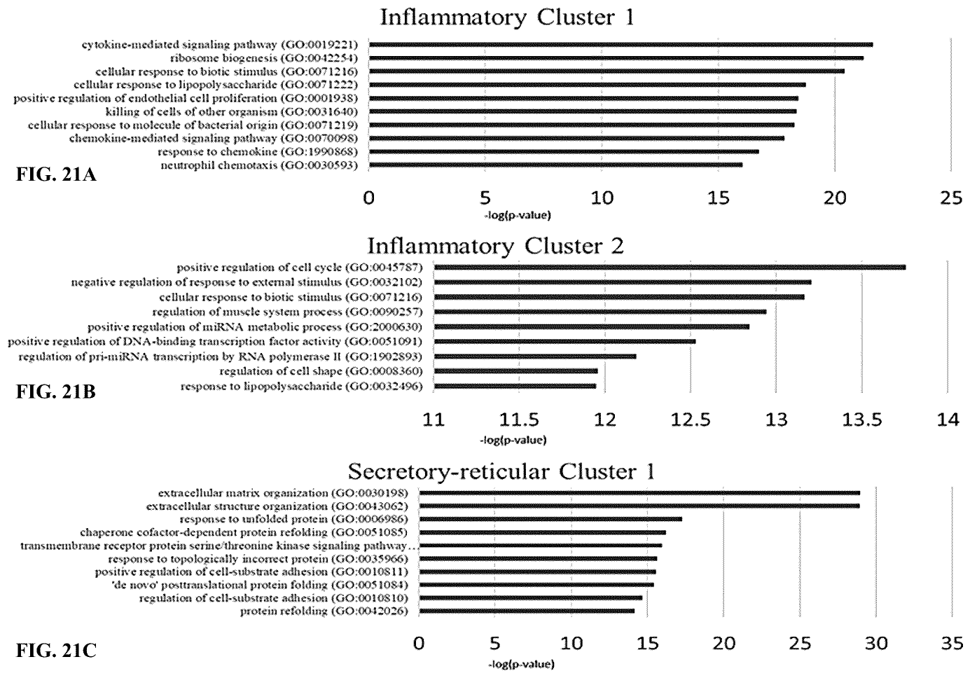

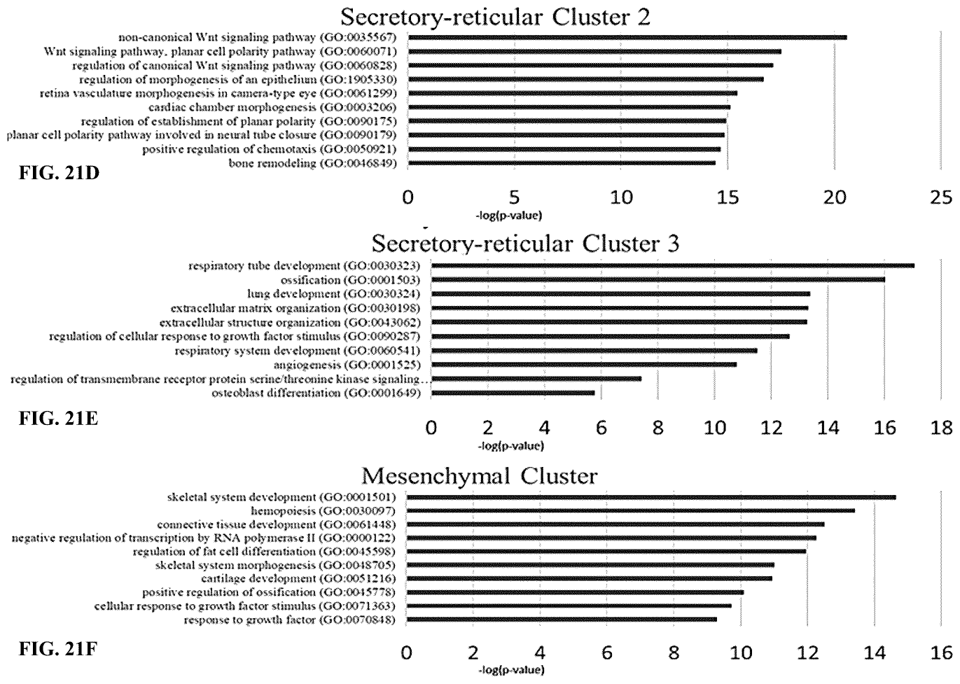

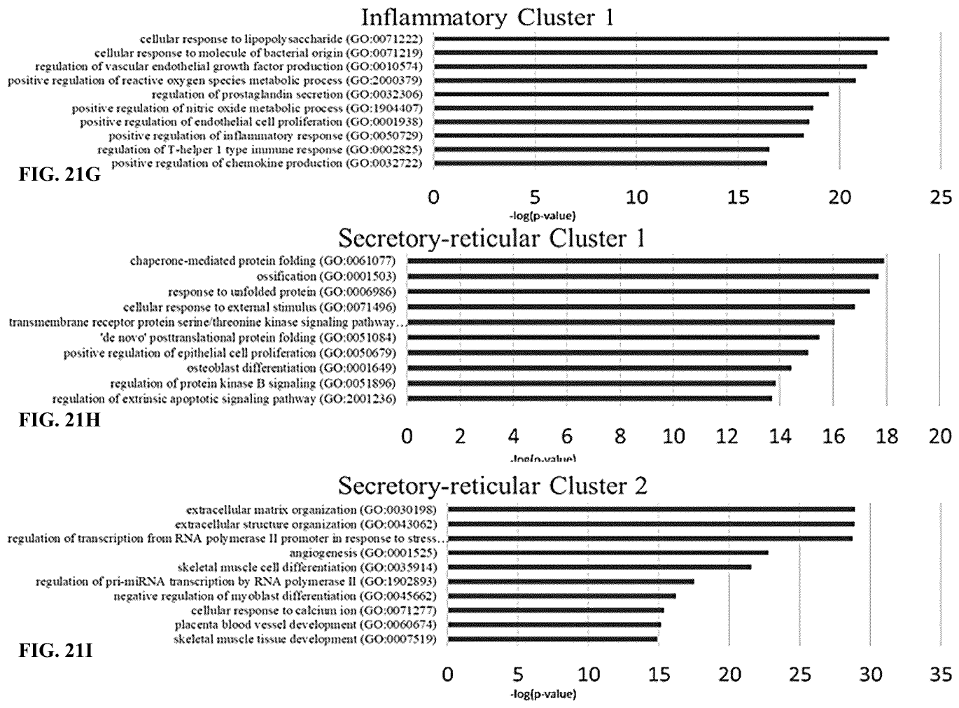

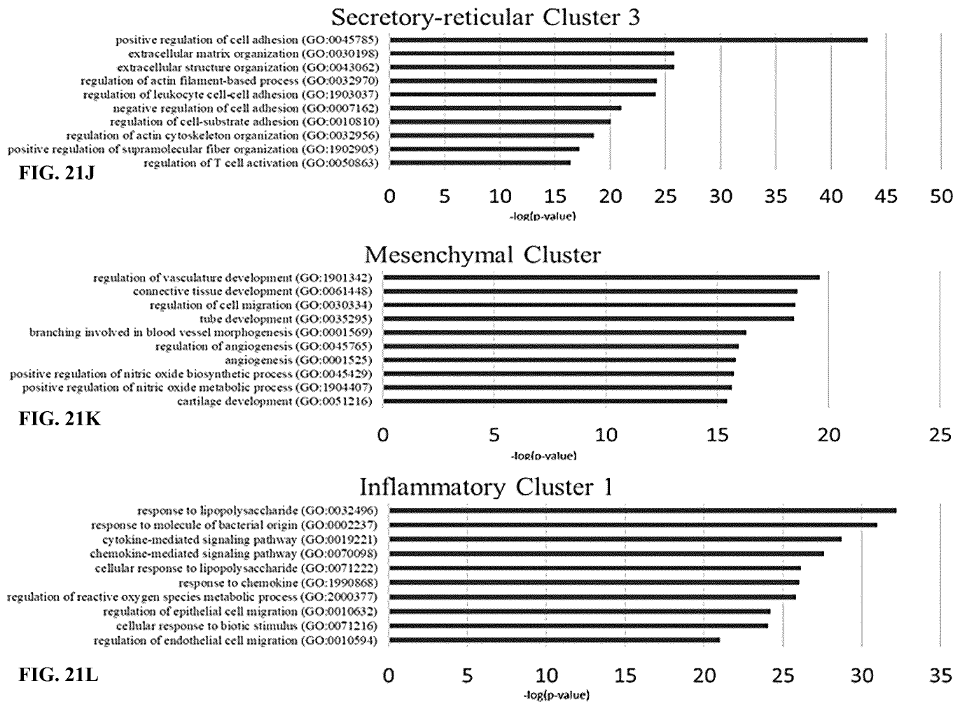

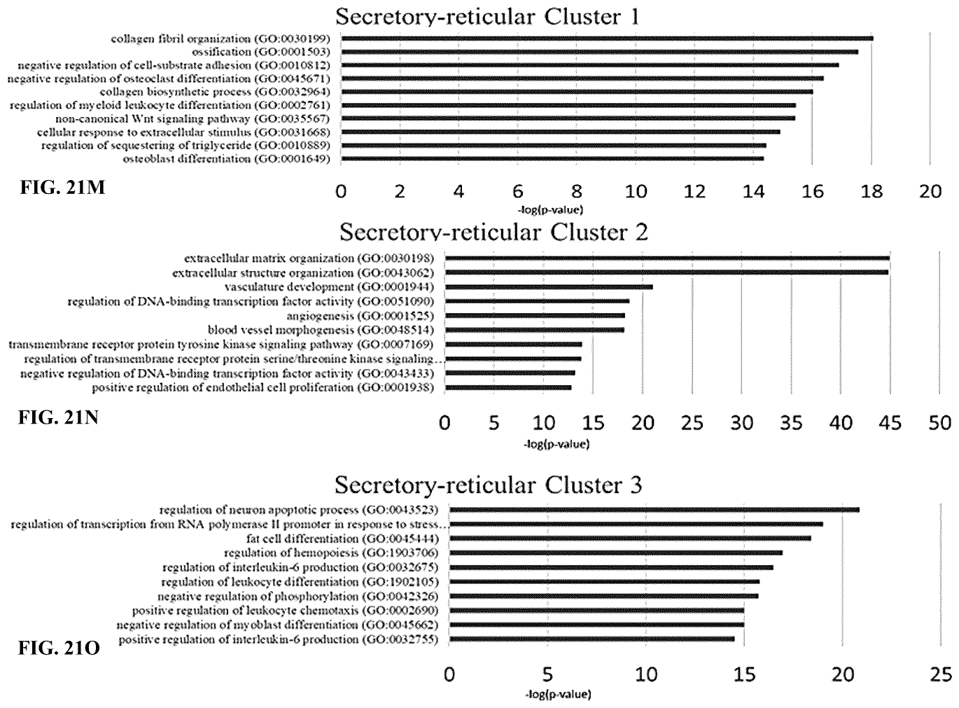

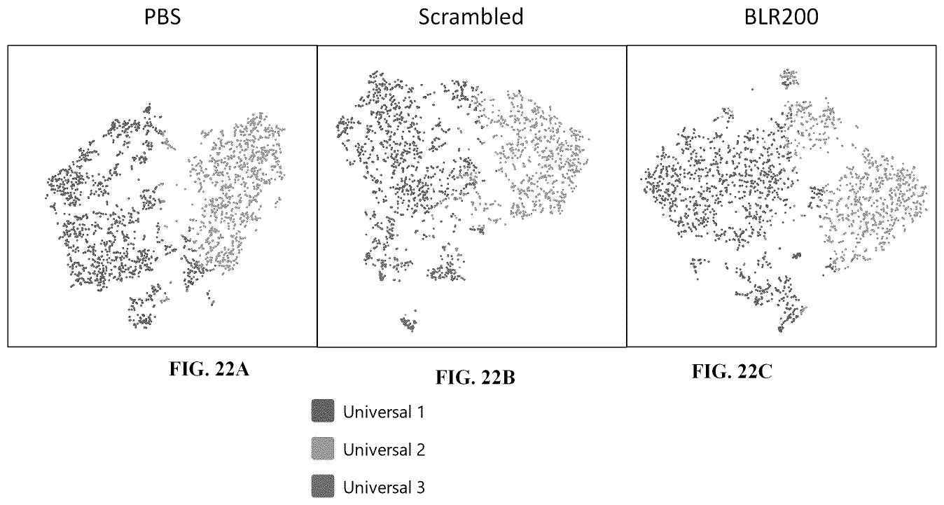

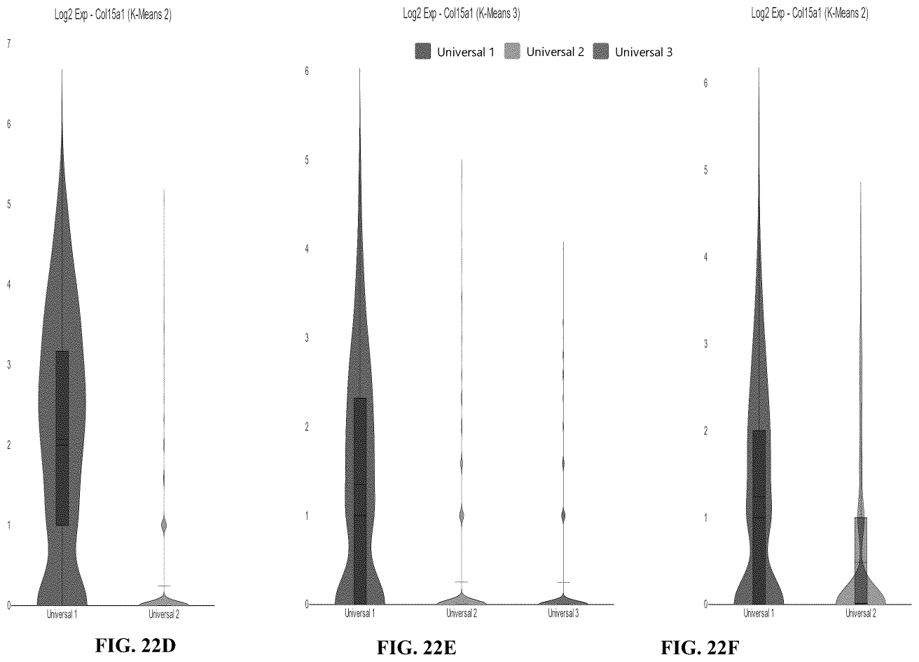

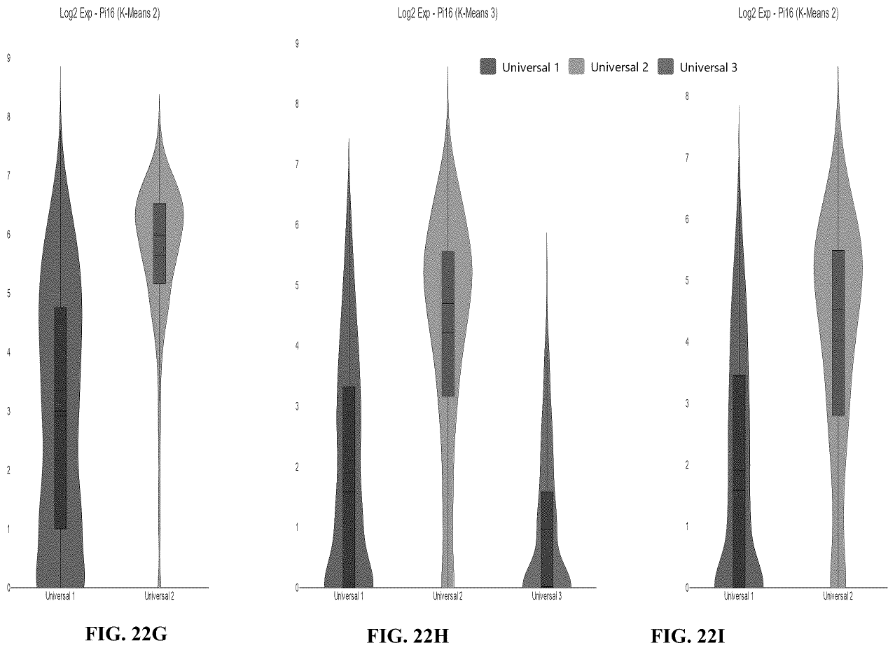

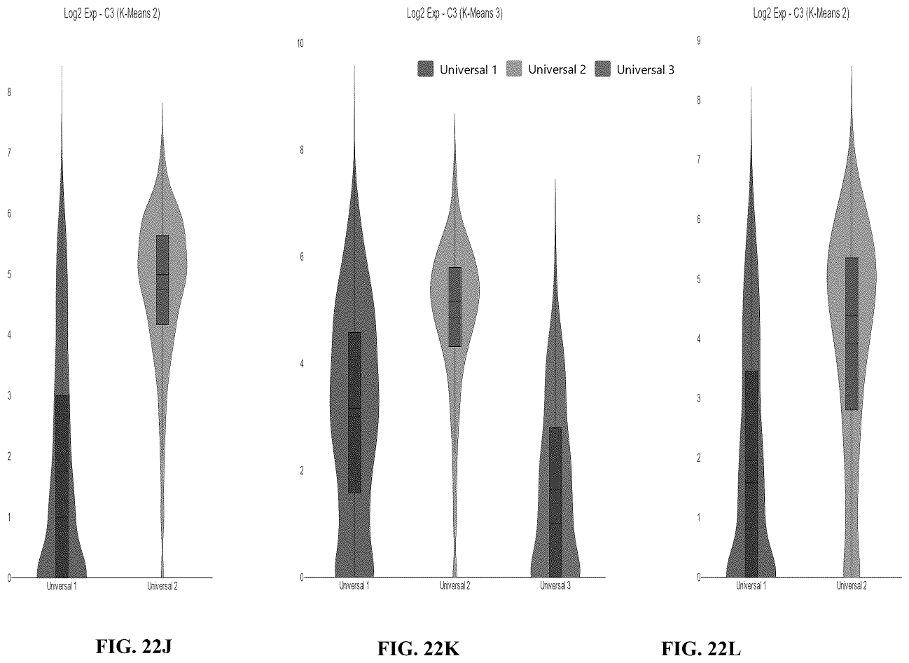

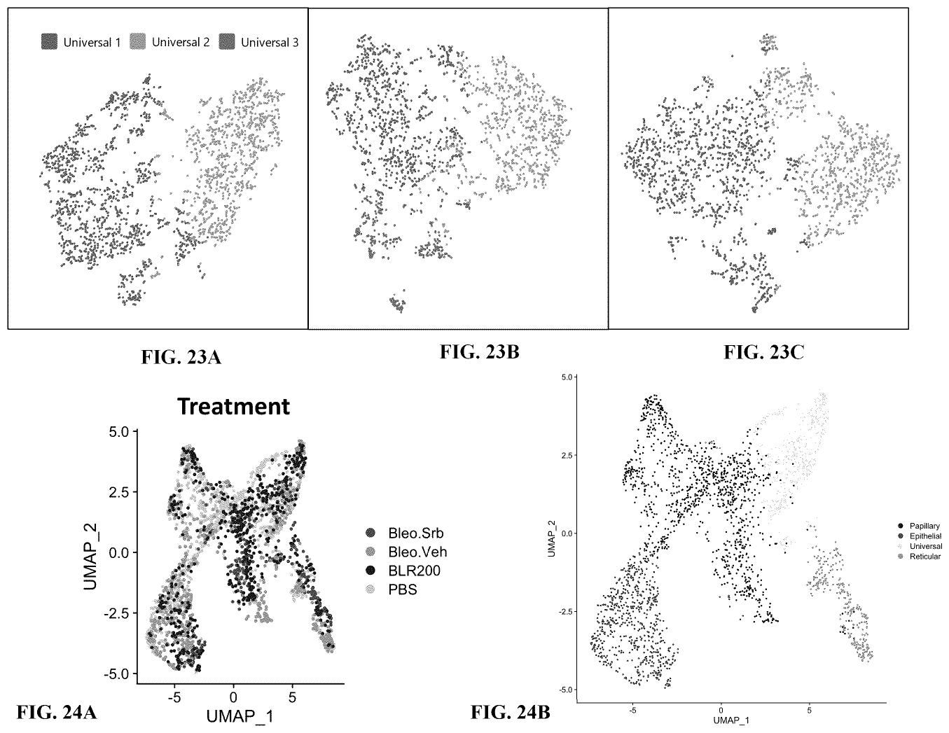

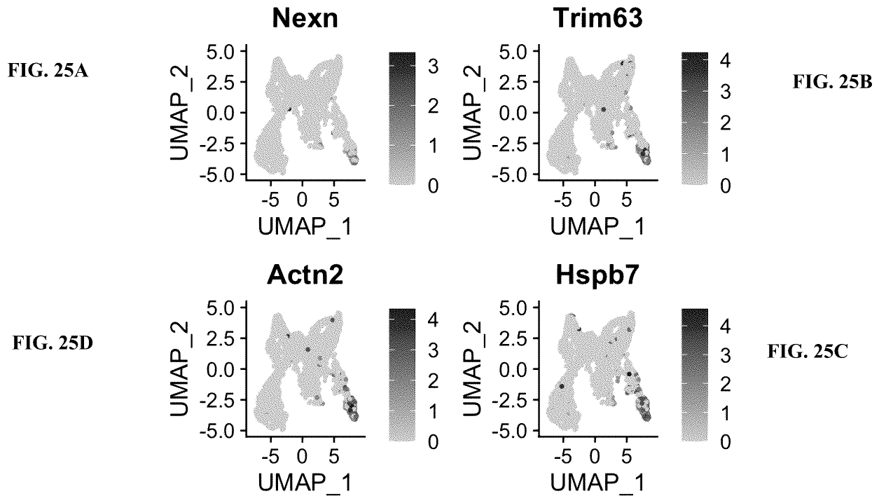

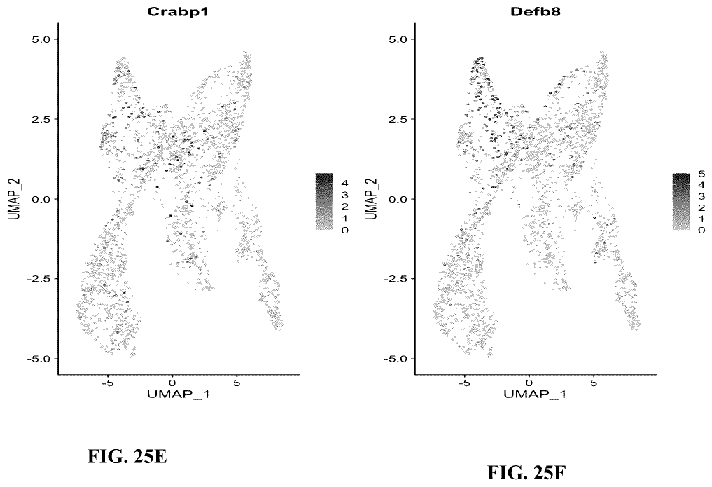

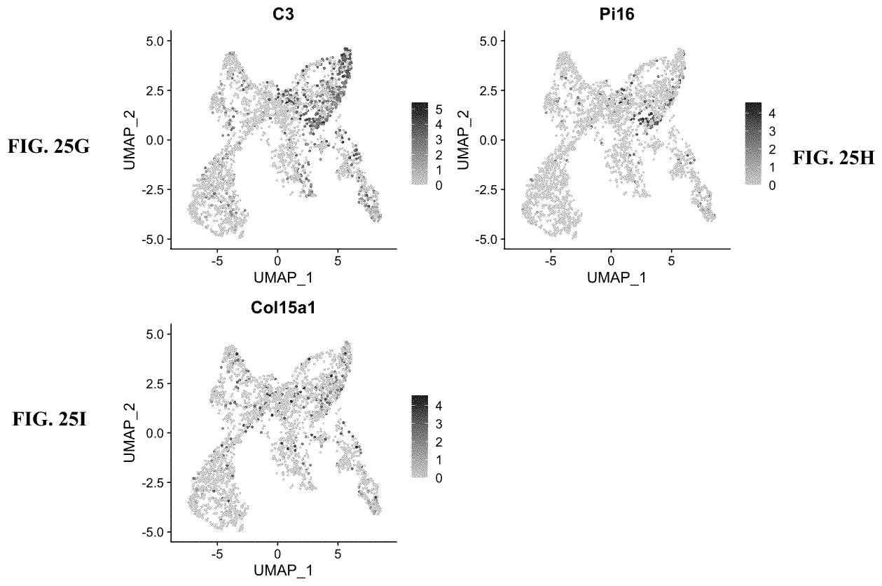

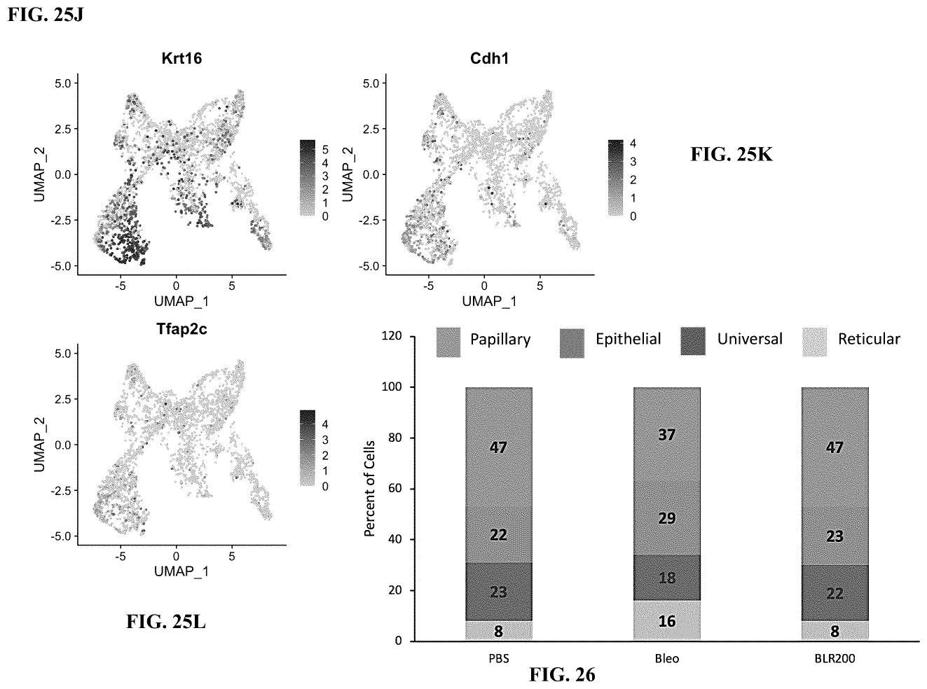

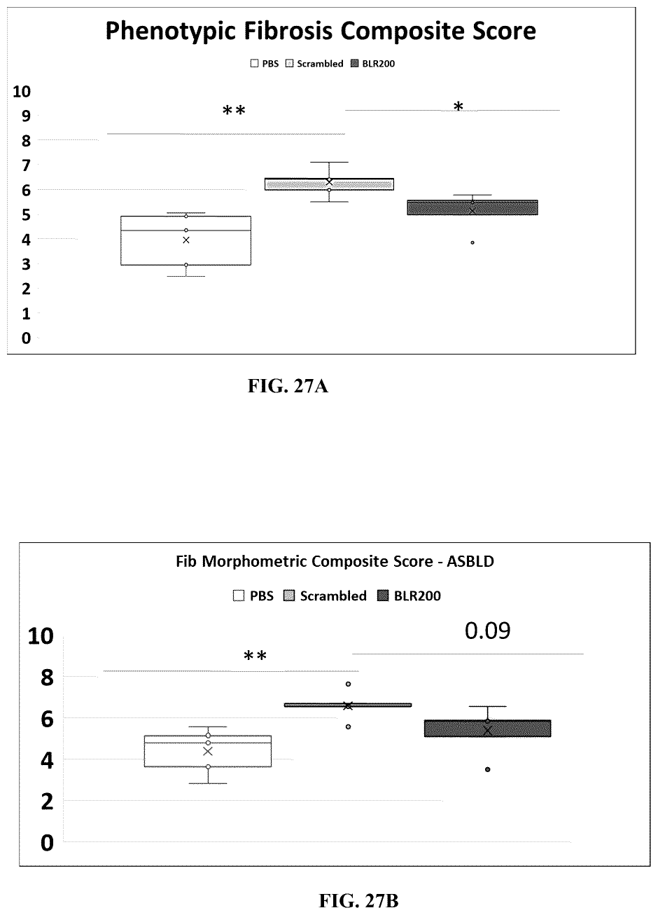

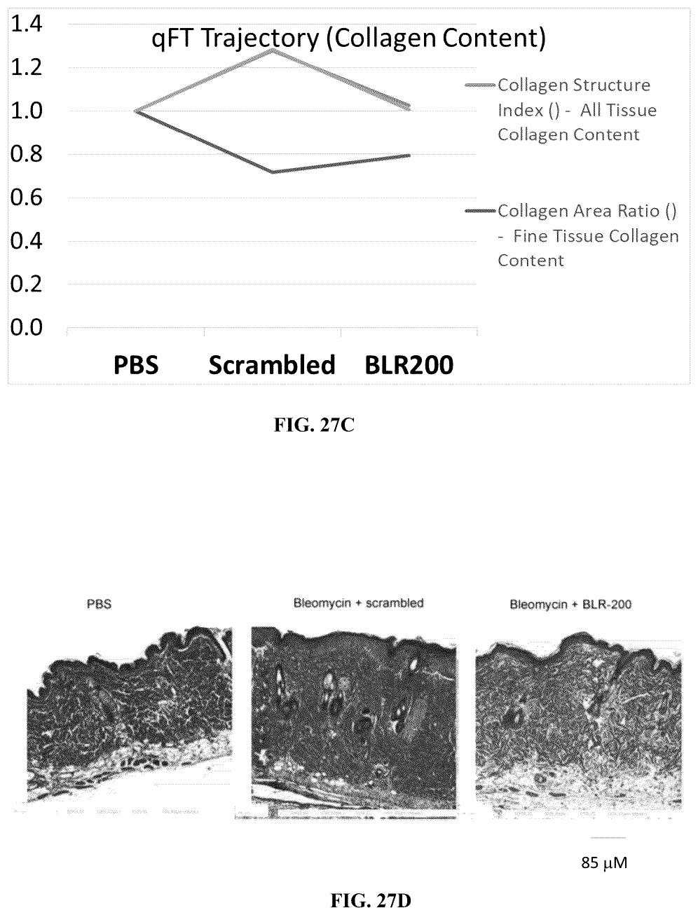

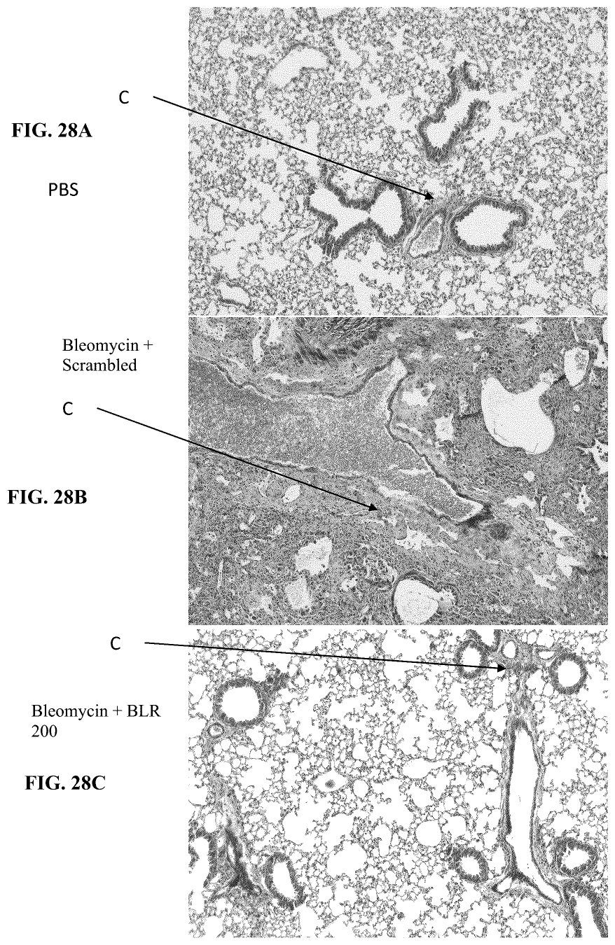

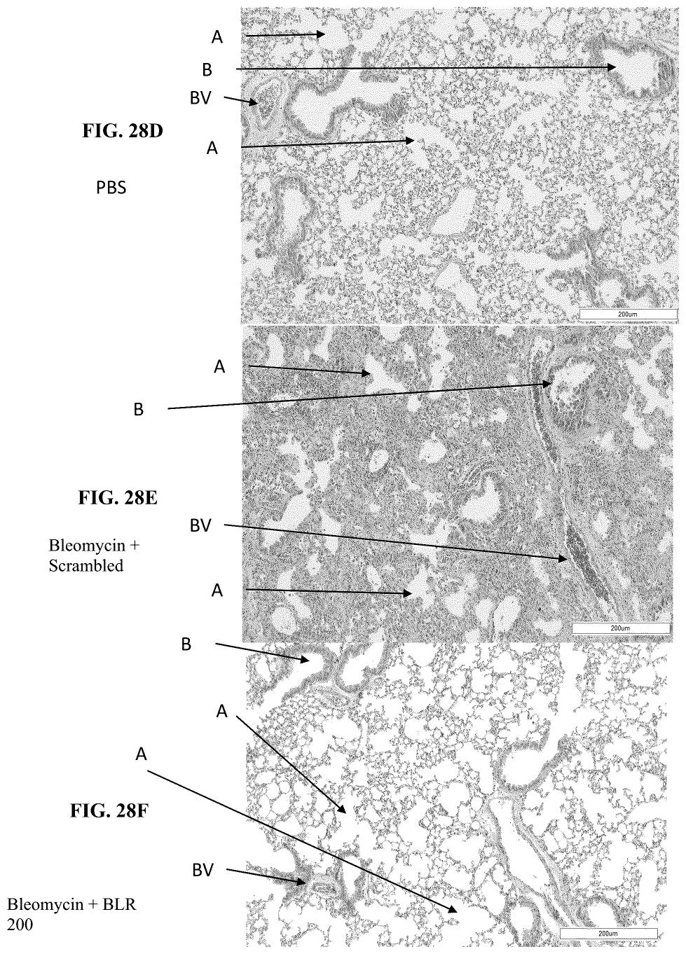

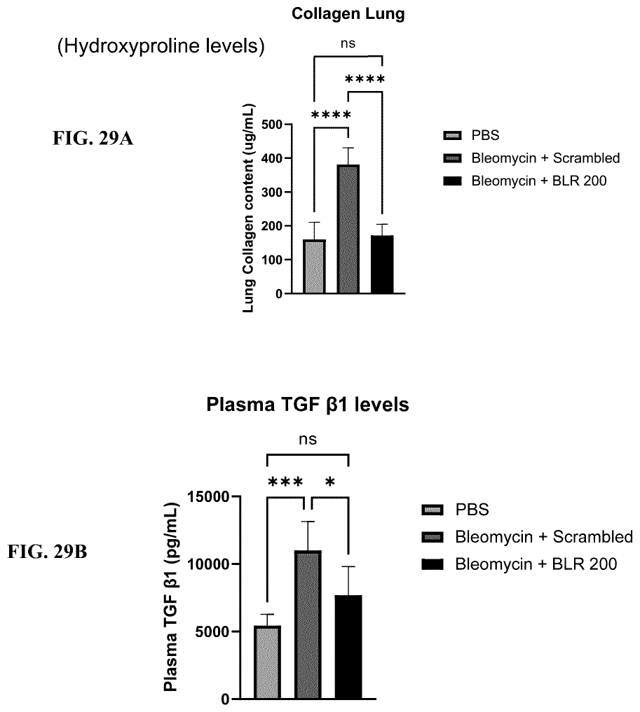

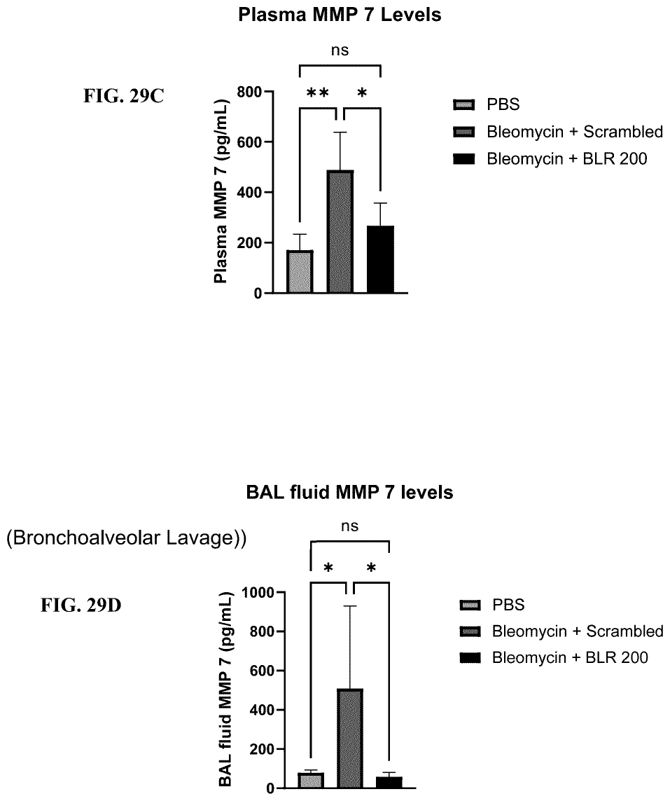

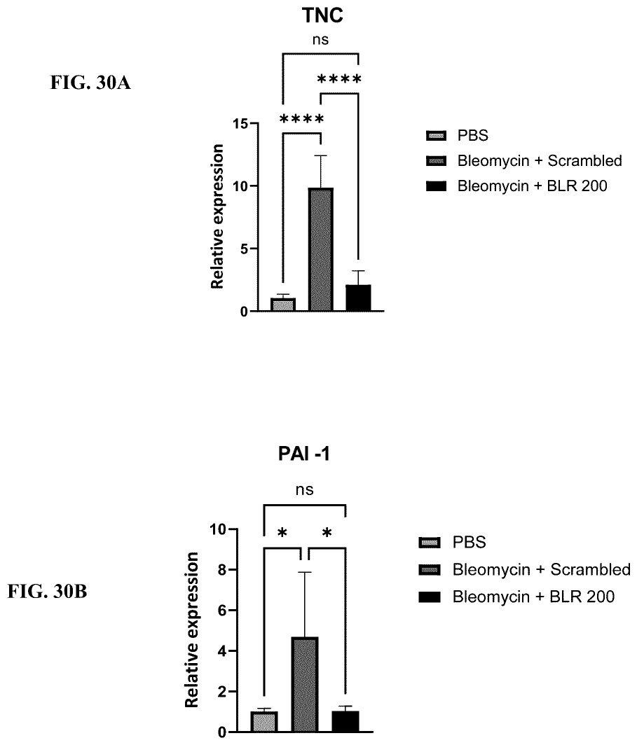

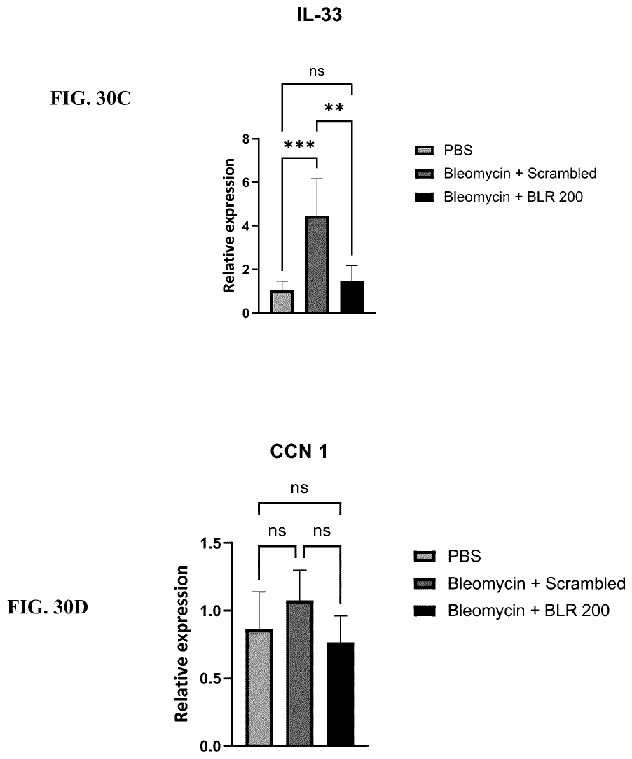

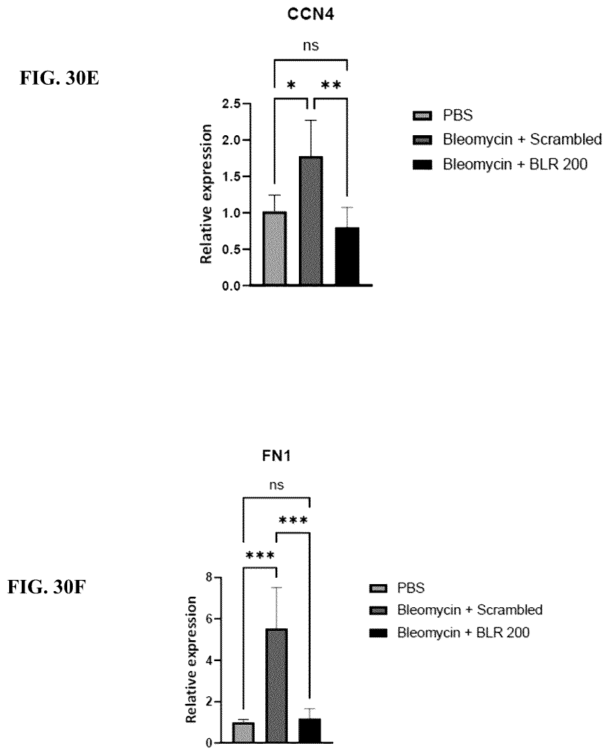

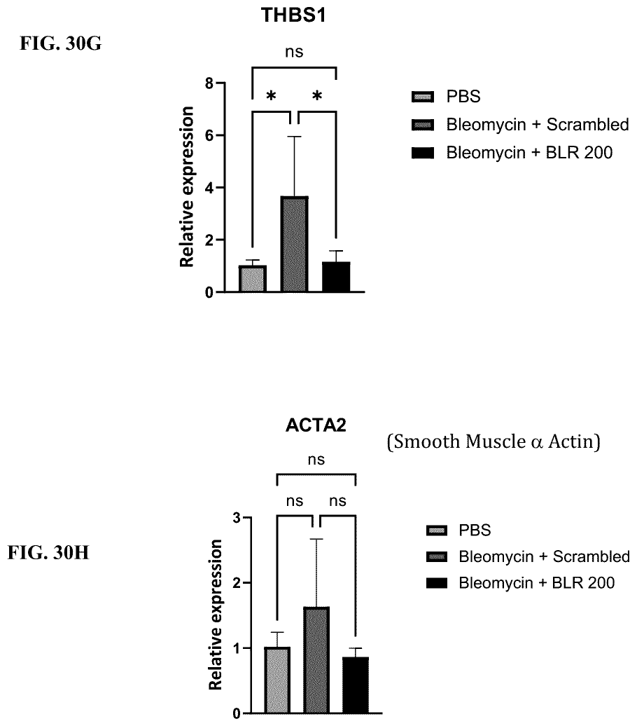

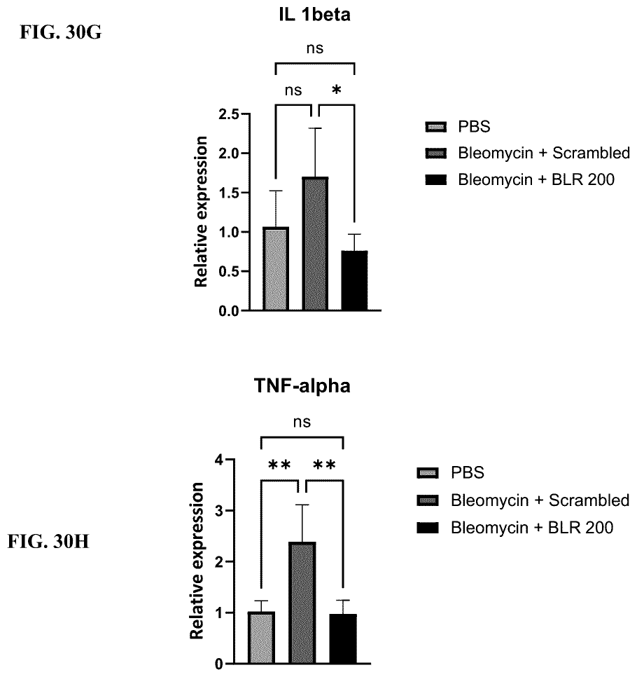

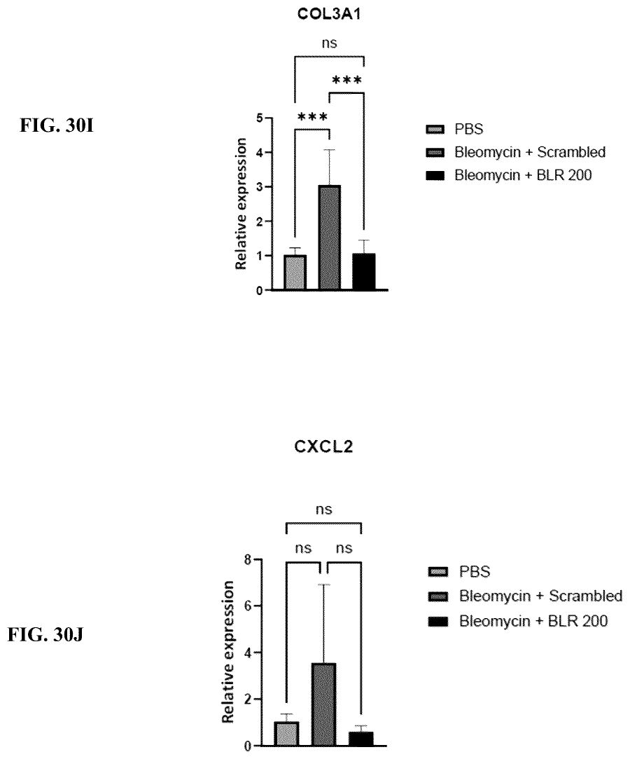

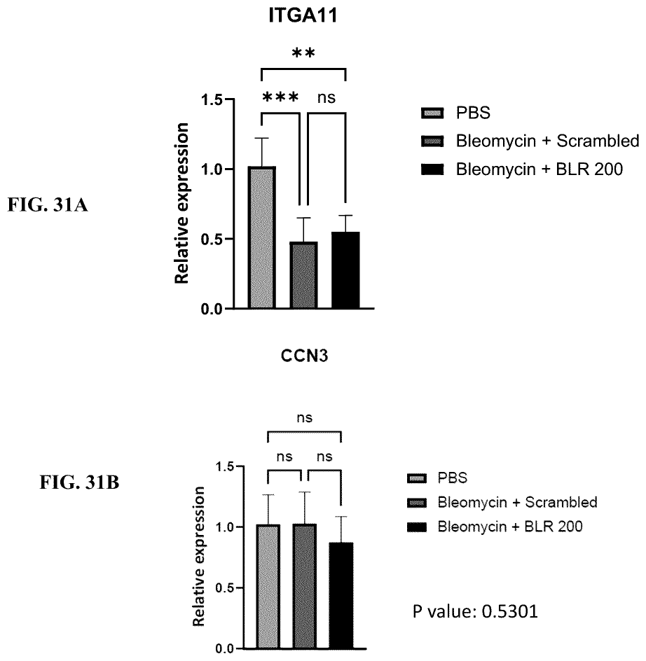

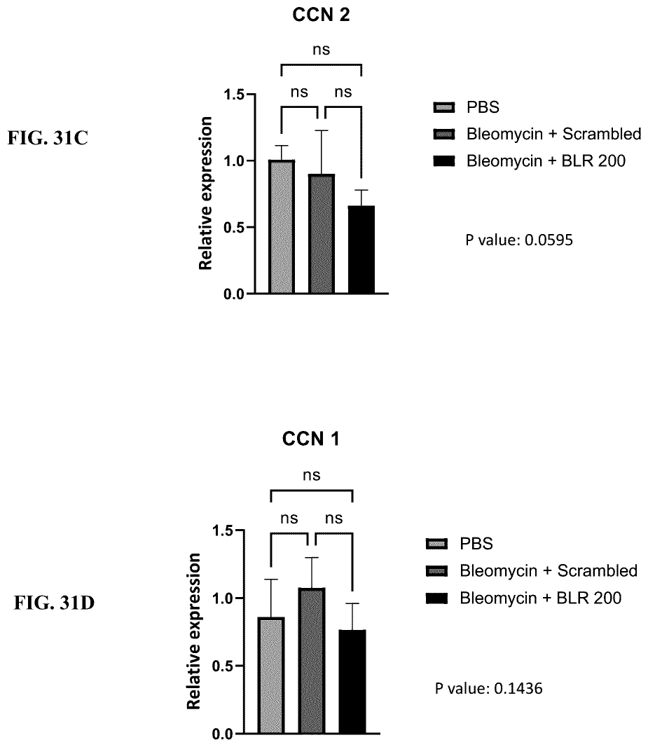

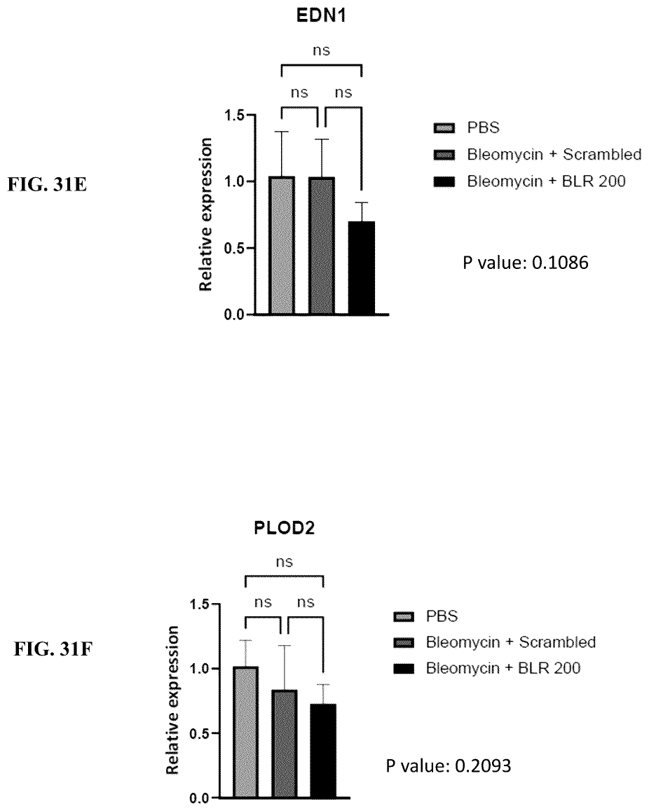

Isolated cells were subjected to scRNA-seq analysis and approximately 2000 cells from each treatment were sequenced. (FIGS. 20A-20C) Unsupervised UMAP clustering of cells reveals that expression of mesenchymal markers Tek, Osr2, and Sfrp2 are not appreciably present in BLR-200 treated mice. Cells are colored based on Log 2 expression of Tek, Osr2, and Sfrp2. (FIGS. 20D-20L) Violin plots showing Log 2 expression of Tek, Osr2 and Sfrp2. BLR-200 treated mice do not appreciably express these mesenchymal markers in any fibroblast subpopulations. Violin plots showing Log 2 expression of (FIGS. 20M-20O) Wnt10b and (FIGS. 20P-20R) Edn1. BLR-200 treatment prevents expression of these pro-fibrotic genes.

FIGS. 21A-210. Functional pathway assessment of fibroblast subpopulations identified in scRNA-seq analysis Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. Graphs showing the top enriched Gene Ontology (GO) terms in each fibroblast subpopulation, sorted by p-value. (FIGS. 21A-21F) PBS, (FIGS. 21G-21K) BLM, and (FIGS. 21L-210) BLR-200.

FIGS. 22A-220 show that BLR-200 impairs activation of collagen lineage fibroblasts. Mice were treated with either PBS (FIGS. 22A, 22D, 22G, 22J, n=3), bleomycin+scrambled peptide (FIGS. 22B, 22E, 22H, 22K, BLM; n=3) or bleomycin+BLR-200 (FIGS. 22C, 22F, 22I, 22L, BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. FIGS. 22A-22C Unsupervised UMAP clustering of cells reveals that expression of collagen lineage fibroblasts COL15A1 and PI16 is impaired in BLR-200 treated mice. Cells are colored based on Log 2(COL15A1 and PI16 expression). Violin plots also reveal that BLR-200 impairs activation of collagen-lineage fibroblasts COL15A1, (FIG. 22F) PI16, (FIG. 22I) C3, (FIG. 22L). Collagen-lineage fibroblasts are universal fibroblasts. 10-day bleomycin/BLR200 collagen 1A2 (GFP)-lineage fibroblast scRNAseq. All 3 clusters are “universal fibroblast”. N=3, 2000 cells (pooled), 30-50K reads/cell.

FIGS. 23A-23C show Unsupervised UMAP clustering of cells reveals of 10-day bleomycin/BLR200 GFP-lineage fibroblast scRNAseq tSNE plot Fibrotic markers TGFB1, ITGAM, ITGB2, ADAM8, SDC3, CCL3/CCL4/CCL6/CCL9, CD14/CD74/IL1b.

FIGS. 24A-24B are UMAP graphs of PBS+Veh.Srb+BLR200 (8 integrated samples).

FIGS. 25A-25K are UMAP graphs of the Cluster Identity detected by established markers, Reticular markers Nexn (FIG. 25A), Trim63 (FIG. 25B), Actn2 (FIG. 25C), Hspb7 (FIG. 25D); Papillary markers: Crabp1 (FIG. 25E), Defb8 (FIG. 25F); Universal markers C3 (FIG. 25G), Pi16 (FIG. 25KH), Col15ai (FIG. 25I); and Epithelial markers: Krt16 (FIG. 25J), Cdh1 (FIG. 25K), Tfap2c (FIG. 25L)

FIG. 26 is a graph showing the Expansion of epithelial and reticular cell populations that are blocked by BLR200 (8 samples).

FIG. 27A is a graph showing the Pharmanest digital analysis of trichrome (study of the structure of collagen in the animal model. FIG. 27B is a graph showing the Fib Morphometric Composite Score—ASBLD. Collagen structure tends to be more uniform in scrambled; reversed in BLR-200 (kurtosity). FIG. 27C is a graph showing the qFT Trajectory (Collagen Content). FIG. 27D is a graph showing the Assembled collagen in PBS, Bleomycin+scrambled, and Bleomycin+BLR-200.

FIGS. 28A-28F are graphs showing the intra-tracheal Bleomycin Study to Determine the Effect of BLR-200 on Lung Fibrosis. Intra-tracheal bleomycin injections on Animals—C57 BL/6 J mice (Male), Age—6 weeks were conducted. BLR 200 and Scrambled, negative control peptide injections (intraperitoneal) were conducted on day 1 through day 20 in three- or two-day intervals. On day 22, Blood, BAL fluid and Lung collection and were Trichrome stained. FIGS. 28A-28C show the Effect of BLR-200 on Tissue Histology, PBS (FIG. 28A), Bleomycin+Scrambled (FIG. 28B), Bleomycin+BLR 200 (FIG. 28C), showing C: Collagen deposition. FIGS. 28D-28F show the Effect of BLR-200 on Tissue H&E staining in PBS (FIG. 28D), Bleomycin+Scrambled (FIG. 28E) and Bleomycin+BLR200 (FIG. 28F) showing A: Air spaces of alveolus, B: Respiratory Bronchiole, BV: Blood vessel.

FIGS. 29A-29D are graphs showing the Effects of BLR-200 Treatment on Biomarkers of IPF/ILD, Collagen Lung (FIG. 29A, hydroxyproline levels), Plasma TGF-B1 levels (FIG. 29B), plasma MMP 7 levels (FIG. 29C), and BAL fluid MMP 7 levels (FIG. 29D, Bronchoalveolar Lavage).

FIGS. 30A-30J are graphs showing the lung Tissue Changes in IPF Biomarkers by qPCR (mRNA Levels): Effect of BLR200. Relative expression of TNC, PAI-1, IL-33, CCN-1, CCN4

FN1, THBS1, ACAT2, IL 1 beta, TNF-alpha, COL3A1, CXCL2 (FIGS. 30A-30J).

FIGS. 31A-31F are graphs showing the Gene Expression Unaltered by Disease or by BLR-200.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### List of Abbreviations

| | |
|---|---|
| ACTB | Beta actin |
| ACTA2 | Actin alpha 2, smooth muscle (Alpha smooth muscle actin) |
| AKT | Protein kinase B family |
| ALK5 | Activin receptor like kinase 5 (Transforming growth factor |
| | beta receptor 1) |
| AP1 | Activator protein 1 |
| BLM | Bleomycin |
| CCN | Cellular (or central) communication network |
| CDNA | Complementary DNA |
| COL1A2 | Collagen type I alpha 2 chain |
| CSF | Colony stimulating factor |
| CT | C-Terminal |
| CXCL | C-X-C motif chemokine ligand |
| DAB | 3,3′-Diaminobenzidine |
| dsSSc | Diffuse cutaneous systemic sclerosis |
| ECM | Extracellular matrix |
| EDN1 | Endothelin 1 |
| EGR1 | Early growth response 1 |
| ERK | Mitogen-activated protein kinase 1 |
| FACS | Fluorescence activated cell sorting |
| FAK | Focal adhesion kinase (Protein tyrosine kinase 2) |
| FAP | Fibro-adipogenic progenitor |
| HSPG | Heparan sulfate proteoglycan |
| IGFBP | Insulin like growth factor binding protein |
| IL | Interleukin |
| IPF | Idiopathic pulmonary fibrosis |
| ITGA11 | Integrin subunit alpha 11 |
| lcSSc | Limited cutaneous systemic sclerosis |
| LOX | Lysyl oxidase |
| MEK1 | Mitogen-activated protein kinase kinase 1 |
| MGP | Matrix Gla protein |
| mRNA | Messenger ribonucleic acid |
| OSR2 | Odd-skipped related transcription factor 2 |
| P4HA1 | Prolyl 4-hydroxylase subunit alpha 1 |
| PAI-1 | Plasminogen activator inhibitor 1 |
| PI3K | Phosphatidylinositol-4,5-bisphosphate 3-kinase |
| qPCR | Quantitative polymerase chain reaction |
| RAC1 | Ras-related C3 botulinum toxin substrate 1 |
| Rna18s | RNA 18s ribosomal 5 |
| scRNA-seq | Single-cell RNA-sequencing |
| SFRP2 | Secreted frizzled related protein 2 |
| SOX2 | SRY-box transcription factor 2 |
| SPARCL1 | Secreted protein acidic and cysteine rich like 1 |
| SRC | Sarcoma proto-oncogene, non-receptor tyrosine kinase |
| SSc | Systemic Sclerosis |
| TAK1 | Mitogen-activated protein kinase kinase kinase 7 |
| TAZ | Transcriptional coactivator with PDZ-binding motif |
| TEK | TEK receptor tyrosine kinase, endothelial |
| TGFβ | Transforming growth factor beta |
| TSP | Thrombospondin |
| VWC | Von Willebrand Factor C Domain Containing |
| WNT10B | Wnt (Wingless-type MMTV integration site) family |
| | member 10B |
| YAP1 | Yes associated protein 1 |
| αSMA | Alpha smooth muscle actin |

### DESCRIPTION OF EMBODIMENTS

Generally speaking, the present disclosure provides innovative methodology and related systems and kits for diagnosing or monitoring various diseases associated with abnormal cell proliferation, inflammation, and fibrosis. Without being bound by theory, there are important biomarkers, expression levels, and precursors for fibrotic conditions. The present disclosure also provides methods for diagnosing, monitoring, staging or detecting new therapeutic agents that treat fibrotic conditions and may mimic or are therapeutically similar to BLR-200.

The term “diagnosis” is used herein to refer to the identification or classification of a molecular or pathological state, disease or condition. For example, “diagnosis” may refer to the identification of a particular type of fibrotic condition associated with a disease. “Diagnosis” is, for example, by pathological or biomarker criteria, or molecular features (e.g., one or a combination of specific genes or subtypes characterized by expression of proteins encoded by said genes) and may refer to the classification of a specific subtype of fibrotic condition.

The term “treat” means decrease, suppress, attenuate, diminish, arrest, or stabilize the development or progression of a disease/disorder associated with a fibrotic condition.

The diagnostic can also be used for patient selection for clinical trials in regards to the stage of the disease and response to therapy. The duration to treat fibrotic condition may be a long period of time, measuring serum and nucleic acid measurements that could be upregulated. Using the diagnostic and screening methods disclosed herein improves the efficiency of clinical trials.

A method of diagnosing a disease state or condition comprises using a gene expression pattern or a protein expression pattern in a fibroblast cell, a progenitor cell, a stem cell, a myofibroblast cell, or a specific myofibroblast population with specific markers to determine early onset of a diseased state, a progression of the disease state, or a regression of the disease state in response to a therapeutic treatment; whereby the disease state is cancer, fibrosis, autoimmune, scleroderma, or systemic fibrosis. The gene expression or protein expression pattern is in cxcl13, Csf2, Il33, IL6, Cxcl2 and the inflammasome NLRP-3 including IL-18, tLr-2, Il1r1, Il1r2, wnt10b or Edn1.

A method of screening a drug or a therapeutic for a disease, comprises: using a gene expression pattern or a protein expression pattern in a fibroblast cell, a progenitor cell, a stem cell, a myofibroblast cell, or a specific defined population of myofibroblasts carrying specific markers; examining the drug or the therapeutic on the protein expression changes and the gene expression changes over a period of time; and selecting the drug or therapeutic to treat the disease based upon a threshold change in the protein expression or gene expression. The drug or therapeutic suppresses myofibroblast differentiation.

The source of material for measurement, diagnosis, or determination of the identified markers can be from tissue, or separated cells for example by biopsy, and or body fluids including whole blood, serum, or plasma, lung (bronchoalveolar lavage), or other body cavity washes. The methods can consist of immunoassays (e.g., ELISA), RT-PCR, single cell RNA sequencing, proteomics, transcriptomics, metabolomics, and others.

One embodiment this technology provide a much needed better determination of patient selection for clinical trials and the likelihood of success for that patient; in those patients in clinical trials and others receiving approved drugs, it will provide early surrogate markers of the response to a given therapy, allowing determination of success vs failure and/or adjustment of drug dose for improved response. Part of this is the ability to better stage disease progression, and/or determine the prognosis for further disease progression versus resolution.

The diagnosis for fibrotic conditions comprises the expression and contribution of CCN proteins in dermal fibrosis, representing or modeling scleroderma (Ssc) and systemic fibrosis,) and determining if therapeutic peptides based on CCN3 represent a novel anti-fibrotic strategy. Since their discovery in the early 1990s, CCN proteins have been shown to play a role in a wide range of cellular processes including: cell adhesion, migration, proliferation, differentiation, survival, and apoptosis (Lau, 2016; Perbal, 2004). Thus, it is not surprising that they have been implicated in several pathological conditions, including: inflammation, retinopathy, cancer, and fibrotic conditions such SSc (Leask, 2020b; Riser et al., 2015). SSc is a chronic disease characterized by vascular abnormalities, dysregulated inflammation, and diffuse fibrosis of the skin and internal organs (Varga & Abraham, 2007). Although much progress has been made in deciphering the complex pathophysiology of SSc, there is still no viable disease-modifying treatment, and patients often experience significant morbidity and mortality (Cutolo et al., 2019). In SSc, CCN1 and CCN2 are upregulated in fibrotic lesions of early-onset patients with diffuse cutaneous disease (Holmes et al., 2003; Quesnel et al., 2019). CCN1 likely plays a role in mediating the formation of organized collagen fibres and may contribute to early inflammatory changes (Quesnel et al., 2019). Fibroblast-specific expression of CCN2 plays a role in promoting a pro-fibrotic microenvironment through the recruitment and sustained activation of myofibroblasts (Liu et al., 2011; Tsang et al., 2019). On the other hand, CCN3 is reciprocally regulated to CCN1 and CCN2 in multiple cell types, and has anti-fibrotic effects opposing CCN2 in renal fibrosis (Riser et al., 2014). This prompted an early stage drug development company, to develop a proprietary CCN3-based peptide, BLR-200, that mirrors the anti-fibrotic effects of full length CCN3 (U.S. Pat. No. 9,114,112B2, 2015). Thus, targeting the activity and regulation of CCN1 and CCN2 indirectly, for example, by using BLR-200, may represent a novel therapeutic and diagnostic approach for SSc.

BLR-200 Includes a Peptide Sequence Found in CCN3

Despite substantial available evidence of CCN protein involvement in fibrosis, there is still a considerable gap in knowledge. The exact mechanisms by which CCN proteins are regulated, and function in vivo remains elusive. Many studies on CCN protein activity have been conducted in vitro using 2-dimensional cell culture models. Although these studies can be useful to decipher simple regulatory mechanisms, they often lack significance in a biological context, as CCN protein activity is crucially dependent on the 3-dimensional composition of the cellular microenvironment (Leask, 2020b). It is imperative to use animal models as well as the latest techniques of analysis to truly understand the physiological function of this unique family of matricellular proteins. Furthermore, few studies have focused on the specific anti-fibrotic role of CCN3 in dermal fibrosis. This diagnostic for fibrotic conditions fills some of those gaps in knowledge and serves to create new inventions to prevent, treat and diagnose disease, SSc and other.

Regulation of CCN Proteins in Dermal Fibroblasts

The first embodiment comprises the reciprocal regulation of CCN1, CCN2, and CCN3 in human dermal fibroblasts in an in vitro model of fibrosis. In this series of embodiments, TGFβ1 was used to stimulate human dermal fibroblasts, and simultaneously monitored the mRNA and protein expression of CCN1, CCN2, and CCN3. Also used were chemical signal transduction inhibitors to identify common pathways mediating the effects of TGFβ1 on CCN1, CCN2, and CCN3 expression. The embodiment comprises mRNA expression of CCN1 and CCN2 are reciprocally regulated to CCN3 in response to TGFβ1 in human dermal fibroblasts. Furthermore, the embodiment comprises CCN1/CCN2 mRNA expression and CCN1/CCN2 protein levels are induced via a similar pro-adhesive pathway involving FAK, MEK1, TAK1, and YAP1. Conversely, CCN3 mRNA is reduced by TGFβ1 through a divergent pathway, that does not involve MEK1, YAP1, or TAK1. These embodiments are consistent with literature showing that CCN1 and CCN2 are expressed in a yin/yang fashion opposite to CCN3 (Abd El Kader et al., 2012; Riser et al., 2010, 2015). These embodiments contribute to the overall understanding of CCN protein regulation and provide implications for CCN protein regulation in dermal fibrosis in vivo. These embodiments are also consistent with the notion that non-canonical pro-adhesive TGFβ signaling promotes a fibrogenic response in fibroblasts (Chagour, 2020; Leask, 2020a). Moreover, the ability of a YAP1 inhibitor to prevent TGFβ1-induced CCN1 and CCN2 expression also sheds new light on the involvement of YAP1 in mediating the pro-fibrotic effects of TGFβ in dermal fibroblasts.

An unexpected problem encountered during this embodiment was the inability to detect CCN3 protein by Western blot, either in the presence or absence of TGFβ1. However, CCN3 protein was detectable in NCI-H295R cells, a tumor cell line that expresses CCN3 (Gazdar et al., 1990; Kyurkchiev et al., 2004), indicating the validity of the methods. Based on this, and previous literature, CCN3 protein is not basally expressed in proliferating human dermal fibroblasts. Previous studies have shown that low levels of intracellular CCN3 are produced in fibroblasts in cultures, however, in growing cells the expression of CCN3 protein is quickly downregulated (Scholz et al., 1996). To examine the potential role of CCN3 in regulating fibrogenic responses, it was necessary, therefore, to generate CCN3-overexpressing human dermal fibroblasts using lentiviral transduction.

In CCN3-overexpressing human dermal fibroblasts, the ability of TGFβ1 to induce CCN2 protein expression was significantly impaired. Consistent with previous reports showing that CCN2-deficient dermal fibroblasts retained TGFβ-responsiveness in vitro (Liu et al., 2011), CCN3 overexpression had no significant effect on the ability of TGFβ1 to induce mRNA expression of the pro-fibrotic biomarkers endothelin-1 and integrin alpha 11. Since in vivo loss or inhibition of CCN2 expression severely impairs fibrogenesis, including myofibroblast activation, in a TGFβ-independent manner (Chen et al., 2006; Holmes et al., 2001), studying the full activity of CCN proteins in animal models is imperative.

In this embodiment, the data from this embodiment provide valuable insights into the coordinated and opposite regulation of CCN1, CCN2, and CCN3 in human dermal fibroblasts. Furthermore, it was shown that CCN3 comprises inhibitory effects on CCN2, thus supporting in vivo testing of the anti-fibrotic effects of CCN3, for example, with CCN3-based peptides.

CCN3-Based Peptides in an Animal Model of SSc Dermal Fibrosis

One embodiment of the most representative murine models of SSc is the bleomycin-induced model. In the bleomycin-induced model, daily subcutaneous bleomycin injection induces an early inflammatory response characterized by the presence of mononuclear and mast cells, resembling early inflammatory changes in SSc (Yamamoto et al., 1999). Early immune infiltration in the lesional dermis results in an inflammatory microenvironment that drives fibrosis. This inflammatory response, however, is short-lived and diminishes after 1-2 weeks of daily injection, at which point, progressive fibrosis occurs in the dermis. After 3-4 weeks of daily injection, dermal thickness increases, with excessive accumulation of collagen and appearance of αSMA-expressing myofibroblasts, mimicking the fibrotic features of lesional SSc fibrosis (Yamamoto, 2017). Thus, the bleomycin-induced model provides a valuable tool to investigate fibrotic changes at multiple stages that resemble those seen in patients with SSc.

In this embodiment, the effect of the CCN3-based peptide, BLR-200, in the bleomycin-induced model at two separate timepoints. The first embodiment comprises the ability of BLR-200 to prevent progressive dermal fibrosis 28 days post-initiation of bleomycin injection. This embodiment comprises the overall anti-fibrotic ability of BLR-200. The second embodiment comprises the ability of BLR-200 to prevent inflammatory changes 10 days post-initiation of bleomycin injection. At this timepoint, the effect of BLR-200 on pro-inflammatory and pro-fibrotic responses to bleomycin-induced fibrosis are assessed. Both timepoints comprise anti-fibrotic activity of CCN3-based peptides in SSc dermal fibrosis.

CCN3-Based Peptides Prevent Bleomycin-Induced Fibrosis

In this embodiment, bleomycin or PBS was injected subcutaneously into the flank of mice once daily for 28 days. Bleomycin-treated mice were further divided into two treatment groups, which received intraperitoneal injections of either BLR-200 or a scrambled peptide. After 28 days, dermal tissue was collected and analyzed by histology, qPCR, and proteomics. This embodiment comprises BLR-200 treatment prevented the bleomycin-induced increase in skin thickness and collagen deposition. Furthermore, qPCR analysis comprises BLR-200 prevented expression of mRNAs encoding the collagen the cross-linking genes Plod2 and Lox. Plod2 and Lox genes are involved in promoting stably cross-linked collagen strands and are upregulated in multiple fibrotic disorders (Chen et al., 2018; van der Slot et al., 2003). Next analyzed was the expression of αSMA, the prototypical marker of activated myofibroblasts (Desmouliere et al., 1993). Based on histological and qPCR analysis, BLR-200 treatment prevented the bleomycin-induced increase in αSMA-expressing myofibroblasts in the dermis.

Next embodiment comprises the CCN1 and CCN2 expression via histology and qPCR. The embodiment indicated that BLR-200 effectively prevented the bleomycin-induced increase in Ccn1 and Ccn2 mRNA expression. Similarly, the number of CCN1- and CCN2-positive cells in the dermis were prevented. This embodiment is consistent with our previous finding that CCN3 overexpression inhibited CCN2 protein expression in dermal fibroblasts. Moreover, this embodiment indicates that, in vivo, CCN3 may also have inhibitory effects on CCN1 expression. The embodiment is also consistent with literature showing that loss of CCN2 impairs skin thickening, collagen deposition, and appearance of αSMA-expressing cells in bleomycin-induced fibrosis (Liu et al., 2011, 2014). This embodiment is the first to show that, in an in vivo model of dermal fibrosis, that CCN3-based treatment can regulate CCN1 and CCN2 expression. It is not known whether this effect is direct or indirect, thus mechanistic studies may be employed in the future. For one embodiment, future studies investigating the potential binding partners of BLR-200 might be useful in deciphering this issue. Protein binding assays, including library screening of potential binding partners for BLR-200 could provide valuable information. These experiments would determine if BLR-200 acts by direct binding to CCN1 and/or CCN2, or by binding to other factors that prevent CCN1 and/or CCN2 expression and activity. It is also possible that BLR-200 acts through a completely different anti-fibrotic mechanism.

Collectively, the embodiment comprises that BLR-200 potently impairs bleomycin-induced skin fibrosis, as demonstrated by prevention of dermal thickening, collagen deposition, activated αSMA-expressing myofibroblasts, and expression of CCN1 and CCN2.

The Proteomic Resource Facility (PRF) was used to establish proteomic analysis of skin samples using mass spectrometry-based Tandem Mass Tag. Proteomic analysis of whole skin samples revealed that BLR-200 prevented several proteins involved in ECM organization (fibromodulin, nectin-2, pecam-1) and pro-fibrotic laminin interactions (lama5, lama3, lamb3, lamc2). The use of this embodiment offered valuable unbiased analysis of the dermal proteome and provides further evidence of the anti-fibrotic effect of BLR-200.

Also, this embodiment comprises the expression of the progenitor cell marker SOX2. Previous studies have shown that collagen-lineage dermal fibroblasts, through a SOX2-expressing progenitor cell-like intermediate, contribute to the activated αSMA-expressing myofibroblast population in bleomycin-induced fibrosis (Liu et al., 2014; Tsang et al., 2019). Furthermore, CCN2 is required for recruitment and activation of this SOX2-positive myofibroblast population (Tsang & Leask, 2014). This embodiment indicates that BLR-200 prevented the bleomycin-induced increase in Sox2 mRNA expression and reduced the number of SOX2-positive cells in the fibrotic reticular dermis. This embodiment is consistent with the observation that BLR-200 can prevent activated αSMA-expressing myofibroblasts in the fibrotic lesion. Thus, this embodiment comprises BLR-200 treatment impairs the CCN2-dependent recruitment and activation of collagen-lineage dermal fibroblasts in bleomycin-induced dermal fibrosis.

While BLR-200 did not have a statistically significant effect on the expression of the mechanosensitive transcriptional cofactor YAP1, BLR-200 comprises a biologically relevant effect, as it is repeatable and very nearly statistically significant by standard methods. YAP1 has been shown to respond to and mediate the pro-adhesive signaling pathway in active myofibroblasts (Shi-wen et al., 2021). Since the data also show that expression of YAP1 target genes including Ccn1, Ccn2, and Acta2 (αSMA) were prevented by BLR-200, it further supports and effect of BLR-200 on YAP.

The overall results of the second embodiment demonstrate that therapeutic treatment with a CCN3-based peptide prevents fibrotic changes in the bleomycin-induced model of SSc dermal fibrosis. This embodiment is the first study demonstrating that targeting CCN proteins using a CCN3-based therapeutic approach prevents fibrotic changes in an animal model of dermal fibrosis. These results represent an exciting discovery in the field and warrant further studies to determine the exact anti-fibrotic mechanisms of CCN3 in SSc dermal fibrosis.

For one embodiment, an alternative pre-clinical method to investigate BLR-200's ability to prevent SSc fibrosis comprises testing the peptides and other drugs or pharmaceuticals in a 3-dimensional cell culture model of human SSc. Advancements in 3-dimensional cell culture technologies have made it possible to develop advanced physiologically relevant 3-dimensional models of SSc in vitro (De Pieri et al., 2021). A 3D model examining the response of patient-derived SSc fibroblasts to BLR-200 treatment would provide ample valuable information. This experiment would allow examination of the direct effect of BLR-200 on SSc fibroblasts. For instance, one could determine if BLR-200 treatment results trans-differentiation, deactivation, or potentially apoptosis of these fibroblasts. Furthermore, one could test if the effects of BLR-200 on SSc fibroblasts are transient or permanent. All of these insights would have significant clinical impact.

CCN3-Based Peptides Impair Fibroblast Response to the Inflammatory Microenvironment

The final embodiment comprises using unbiased proteomic and scRNA-seq analysis to investigate the ability of BLR-200 to suppress specific early inflammatory and pro-fibrotic responses in bleomycin-induced fibrosis after 10 days. The animal experiments were performed as previously reported, except tissue samples were collected 10 days post-initiation of bleomycin injection.

Analyzed were the overall proteomic changes using mass spectrometry. BLR-200 prevented the bleomycin-induced increase in markers of keratinocyte activation including keratin1, keratin6, keratin14, and keratin15. Keratinocyte activation represents an early inflammatory response in the epidermis of fibrotic lesions, including in SSc (Aden et al., 2008; Nikitorowicz-Buniak et al., 2014). BLR-200 prevented markers of myofibroblast activation including fibrillin-1, dermatopontin, tropomyosin, and myosin light chain 6b (Fujimura et al., 2011; Kissin et al., 2002; Malmstrom et al., 2004; Okamoto & Fujiwara, 2009). Thus, this embodiment comprises BLR-200 inhibits inflammation-driven pro-fibrotic protein expression observed 10 days post-initiation of bleomycin injection. Considering that, in SSc, keratinocyte activation precedes fibrotic changes in the microenvironment and promote myofibroblast activation (Aden et al., 2010; McCoy et al., 2017), it is likely that fibroblasts in the fibrotic lesion are in the early stages of activation after 10 days of bleomycin injection. Given this observation, next embodiment comprises the transcriptomic changes occurring in the activating fibroblasts at this point, and the effect of BLR-200 on these changes.

Thus, the ability of BLR-200 to prevent early inflammatory and pro-fibrotic responses in bleomycin-induced fibrosis, scRNA-seq was used to investigate transcriptomic changes in the activating collagen-lineage fibroblast population. The use of scRNA-seq provides an unbiased approach to decipher fibroblast heterogeneity and identify gene signatures capable of predicting biological activity (Haque et al., 2017). In this, a transgenic mouse line was used to postnatally label Col1a2-expressing resident dermal fibroblasts with membrane-targeted GFP (Bou-Gharios et al., 1996; Denton et al., 2001). This population is known to become activated and contribute to the myofibroblast population in bleomycin-induced fibrosis (Tsang et al., 2019). Thus, labelling these “pre-myofibroblasts” allows for investigation of cellular changes in cells undergoing myofibroblast activation in response to the inflammatory microenvironment. At the conclusion of the experiment, fibroblasts were isolated from the dorsal skin of the mice, and collagen-lineage (GFP+) cells were sorted by FACS. These cells were then subjected to scRNA-seq. As expected, the initial scRNA-seq analysis revealed that collagen-lineage myofibroblasts exhibited considerable heterogeneity in bleomycin-induced fibrosis. Among the treatment groups, identified was 7 different fibroblast subpopulations that were roughly classified into 3 major groups, including an inflammatory subset, a secretory-reticular subset, and a mesenchymal-like subset.

Next, this embodiment comprises the effects of BLR-200 on these fibroblast subpopulations. BLR-200 impaired a bleomycin-induced transcriptomic shift in the inflammatory fibroblast subpopulation (IF1). Upon investigation of the genes responsible for this shift, BLR-200 impaired the bleomycin-induced increase in Il6 and Cxcl2, both of which have been implicated in early fibrogenesis in SSc (Feghali et al., 1992; Johnson et al., 2015). BLR-200 treatment also prevented the bleomycin-induced overexpression of NLRP3 inflammasome markers, including Il18, Tlr2, Il1r1, and Ilr2. Inflammasome activation is an early inflammatory event in fibrogenesis in multiple organs, including the lung, kidney, liver, and dermis (Colak et al., 2021; Hsu et al., 2021; Rastrick & Birrell, 2014). Furthermore, an inflammasome-related gene signature is overexpressed in SSc and may play a role in myofibroblast activation and excessive collagen deposition (Artlett et al., 2011; Martinez-Godinez et al., 2015). Thus, the data supports and enables that BLR-200 can impair early pro-inflammatory changes implicated in creating a pro-fibrotic microenvironment.

Another embodiment comprises BLR-200 preventing the bleomycin-induced increase in Egr1 expression in the secretory-reticular (SC2) fibroblast subpopulation. EGR1, a transcription factor that mediates early cellular responses to injury, is also overexpressed in the fibrotic tissue of SSc patients (Bhattacharyya, Wu, et al., 2011). Moreover, EGR1 overexpression is implicated in an inflammatory subset of SSc patients, indicating its potential role in mediating early inflammatory events in fibrogenesis (Bhattacharyya, Sargent, et al., 2011). Interestingly, BLR-200 treatment failed to prevent the bleomycin-induced increase in expression of other pro-fibrotic transcription factors, including Fosb and Junb (Avouac et al., 2012). This embodiment is consistent with the notion that BLR-200 has very specific anti-fibrotic effects.

Finally, scRNA-seq analysis supported and enabled that BLR-200 prevented the appearance of a mesenchymal fibroblast subpopulation. This mesenchymal subpopulation was characterized by enrichment of functional pathways involved in development and differentiation (Solé-Boldo et al., 2020). Previous studies have reported that a mesenchymal-like fibroblast subpopulation contributes to dermal fibrosis, and this population may actually be primed to over-respond to fibrotic stimuli (Deng et al., 2021; Taki et al., 2020). Therefore, BLR-200 prevents the appearance of a highly pro-fibrotic subpopulation.

To date, scRNA-seq studies have mainly focussed on dissecting fibroblast heterogeneity in dermal fibrosis (Ascensión et al., 2021; Deng et al., 2021; Philippeos et al., 2018; Solé-Boldo et al., 2020). While this provides useful information, very few studies have investigated this heterogeneity in response to a pro-inflammatory, pro-fibrotic microenvironment. These embodiments contribute to reducing this gap in knowledge. The results also emphasize the important contribution of collagen-expressing dermal fibroblasts to the heterogenous fibroblast subpopulation in bleomycin-induced dermal fibrosis. Furthermore, it supports and enables therapeutic peptides (BLR-200) based on CCN3 impair early pro-inflammatory and pro-fibrotic responses in this collagen-lineage fibroblast population.

The bleomycin-induced model of SSc, while one of the more useful tools for studying SSc fibrosis, is an animal model and is a simplification of a much more complex human disorder. Nevertheless, the findings still provide novel information on the pathogenic mechanisms of fibrosis and will potentially contribute to the overall understanding of mechanisms underlying human SSc.

The data generated represent a valuable starting point for an extensive study aimed at full assessment of the ability of BLR-200 to prevent fibrosis. Since fibrogenesis is a highly spatially and temporally regulated process, investigating multiple different timepoints in the bleomycin-induced model provides greater insight into the exact anti-fibrotic effects of BLR-200. For example, investigating changes at 5 days post-initiation of bleomycin injection would allow us to determine the effect of BLR-200 on the robust inflammatory response that occurs immediately following vascular injury. Furthermore, investigating changes 15 days post-initiation of bleomycin injection would provide insight on BLR-200's effects during the transition from the inflammatory phase to the actively fibrotic phase of the model. A longitudinal study of this nature would likely reveal the contribution and fate of the observed mesenchymal-like fibroblast subpopulation. Moreover, while the scRNA-seq studies provide valuable, novel information on fibroblast heterogeneity in bleomycin-induced dermal fibrosis, investigation of multiple timepoints would greatly build upon characterization of this model. This would have a significant impact on our overall understanding of fibrosis and positively impact pre-clinical testing of other potential anti-fibrotic therapies.

Investigating the effect of BLR-200 on established fibrosis will also uncover valuable therapeutic information. It could focus on the ability of BLR-200 to prevent fibrosis and impair the early response to inflammation. This is clinically important, especially in early dcSSc, when fibrogenesis is actively occurring (Asano, 2018).

### SUMMARY

In the embodiments disclosed, it was shown that in dermal fibroblasts, CCN1 and CCN2 are reciprocally regulated to CCN3 through differential use of non-canonical TGFβ signaling pathways. Furthermore, CCN3-based peptides treat fibrosis in an animal model of SSc dermal fibrosis and impair the ability of collagen-expressing fibroblasts to respond to the inflammatory microenvironment. Thus, the embodiments comprise therapeutic treatment and diagnosis of CCN3-based peptides in treatment of fibrotic disorders, a finding that may have significant clinical impact.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Peptide BLR-200 Blocks and Reverses Bleomycin-Induced Fibrosis

BLR-200 as a novel, small synthetic peptide derived from CCN3 (a known endogenous antifibrotic protein). This example shows that BLR-200 suppresses bleomycin-induced skin fibrosis and to uncover its underlying mechanism.

Materials & Methods.

6-week-old C57BL/6J mice were subjected to bleomycin treatment (subcutaneous injection 0.1 U/100 μl/mouse daily) in the presence or absence of BLR200 (intraperitoneal injection 10 μg/kg every other day) for 21 days. For a reversal of fibrosis experiment, BLR200 treatment was started at d14 of bleomycin treatment and continued until d28. Fibrosis was assessed by histological, and unbiased bulk RNAseq, real-time polymerase chain reaction and spatial transcriptomic analyses.

Results

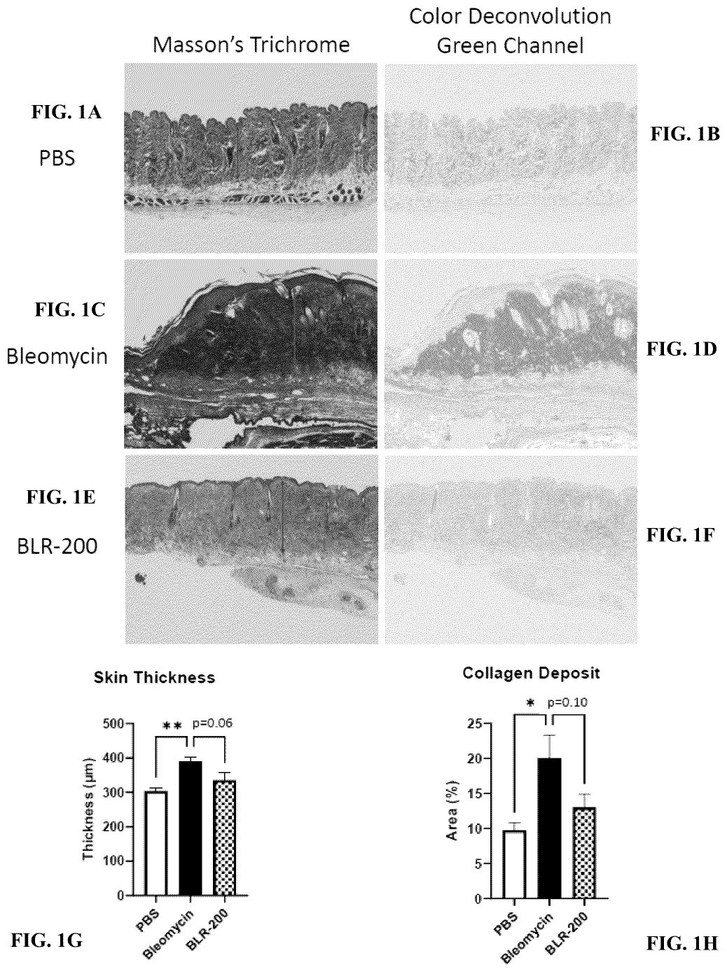

FIGS. 1A-1G show that collagen deposition and thickening of the skin were indistinguishable between BLR-200 treatment and control. Skin thickness was determined by measuring the dermal layer (red arrow). FIGS. 1F-1G show collagen deposition measured by ImageJ. FIGS. 1A, 1C, and 1D show the Masson's trichrome image was deconvolved using color deconvolution plugin in ImageJ. FIGS. 1B, 1F, and 1E show the green channel image represented the collagen fibers. Data was presented as mean±SEM, n=5. Statistical analysis was performed using one-way ANOVA followed by Tukey-Kramer test. *p≤0.05, **p≤0.01

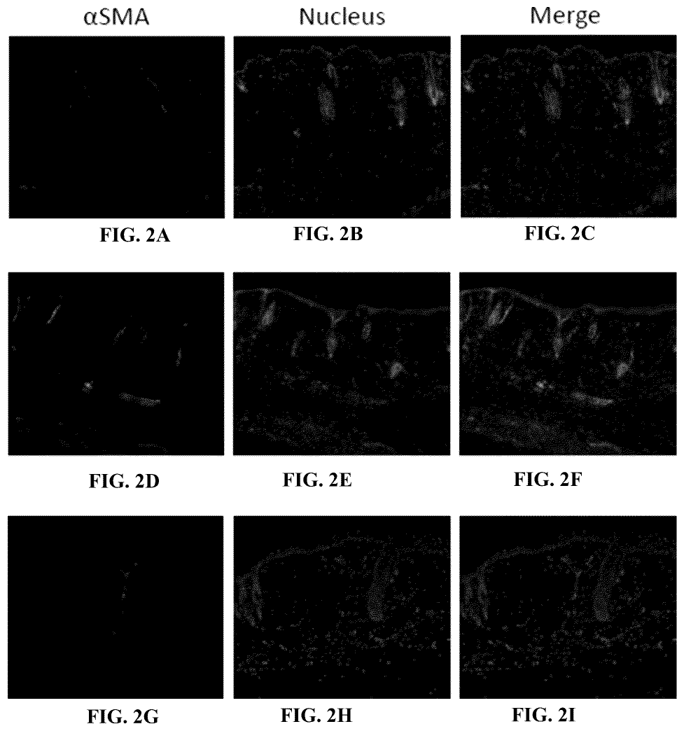

FIGS. 2A-2J show that BLR-200 suppressed myofibroblast differentiation in bleomycin-induced fibrosis at d21. Immunofluorescence was performed using αSMA antibody, a marker of myofibroblast. FIG. 2J is a graph showing the data was quantified by % area of myofibroblasts. Data was presented as mean±SEM, n=5. Statistical analysis was performed using one-way ANOVA followed by Tukey-Kramer test. **p≤0.0.1 FIGS. 2A-2C are healthy mice, FIGS. 2D-2F are diseased mice (i.e. with bleomycin), 2G-2I are bleomycin plus BLR-200).

Table 1 shows BLR-200 suppressed the expression of genes in fibrosis related-clusters induced by bleomycin at d21. Unbiased cluster analysis of bulk RNAseq data was performed using the Database for Annotation, Visualization and Integrated Discovery (DAVID). Clusters relate to fibrosis with the enrichment score and #of genes greater than 1.0 and 10, respectively, were chosen. n=2.

TABLE 1
BLR-200 suppressed myofibroblast differentiation
in bleomycin-induced fibrosis at d 21

| | | | |
|---|---|---|---|
| | Gene | Fold Chance | Fold Chance |
| Gene Name | Symbol | by Bleomycin | by BLR-200 |

Chromatin Binding, Enrichment Score: 2.45, # Genes: 37

| | | | |
|---|---|---|---|
| Yes-associated protein 1 | YAP1 | 8.61 | 2.32 |
| Cbp/300-interacting transactivator, with | CITED2 | 6.43 | 1.1 |
| Glu/Asp-rick carboxy-terminal domain, 2 | | | |
| ELK1, member of ETS oncogene family | ELK1 | 3.69 | −12.5 |
| SMAD family member 3 | SMAD3 | 3.07 | −4.17 |

Metalloprotease, Enrichment Score: 1.35, # Genes: 13

| | | | |
|---|---|---|---|
| A disintegrin and metallopeptidase | ADAM10 | 3.68 | −1 |
| domain 10 | | | |
| A disintegrin-like and metallopeptidase | ADAMTS20 | 11.3 | 1.92 |
| (reprolysin type) with thrombospondin | | | |
| type 1 motif, 20 | | | |
| A disintegrin-like and metallopeptidase | ADAMTS4 | 4.29 | 1.86 |
| (reprolysin type) with thrombospondin | | | |
| type 1 motif, 4 | | | |
| A disintegrin-like and metallopeptidase | ADAMTS9 | 4.46 | −1.19 |
| (reprolysin type) with thrombospondin | | | |
| type 1 motif, 9 | | | |

Cell-Cell Adhesion, Enrichment Score: 1.92, # Genes: 18

| | | | |
|---|---|---|---|
| Integrin alpha E, epithelial-associated | ITGAE | 3.68 | −1 |
| Cadherin 11 | CDH11 | 11.3 | 1.92 |
| Platelet/endothelial cell adhesion | PECAM1 | 4.29 | 1.86 |
| molecule 1 | | | |
| Integrin alpha 6 | ITGA6 | 4.46 | −1.19 |

Wnt Signaling Pathway, Enrichment Score: 1.85, # Genes: 18

| | | | |
|---|---|---|---|
| Frizzled class receptor 6 | FZD6 | 22.4 | −1.40 |
| Wingless-type MMTV integration site family, | WNT4 | 3.07 | 2.49 |
| member 4 | | | |
| Wingless-type MMTV integration site family, | WNT7a | 5.81 | 1.57 |
| member 7A | | | |

Fibronectin Type-III, Enrichment Score: 1.43, # Genes: 15

| | | | |
|---|---|---|---|
| Tenascin C | TNC | 3.13 | 2.39 |
| Fibronectin 1 | FN1 | 5.12 | 2.06 |

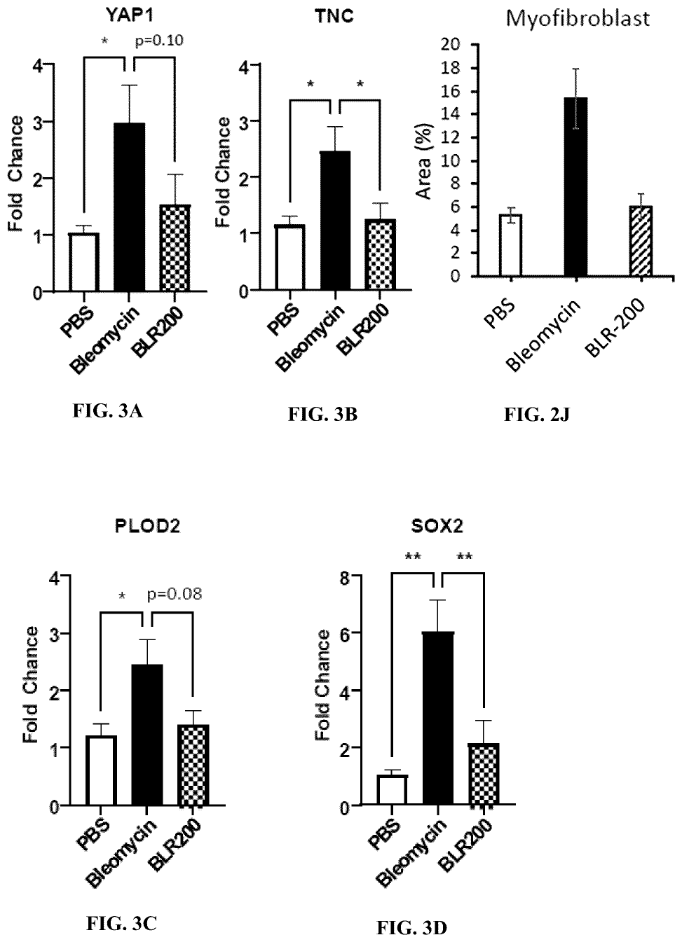

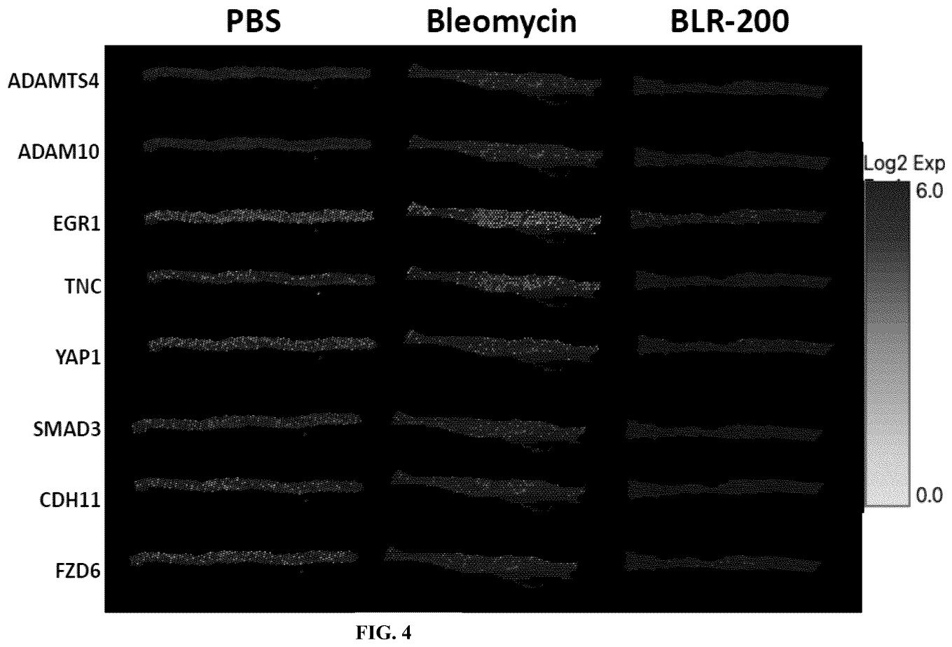

FIGS. 3A-3D show the effect of BLR-200 on the mRNA expression of fibrosis related genes determined by RT-PCR. The mRNA expression of mechanical sensing YAP1, extracellular matrix glycoprotein TNC, collagen crosslinking enzymes PLOD2 and multipotential neural crest stem cell marker SOX2 was indistinguishable between BLR-200 treatment and control. Data was presented as mean±SEM, n=5. Statistical analysis was performed using one-way ANOVA followed by Tukey-Kramer test. *p≤0.05, **p≤0.01.

FIG. 4: Visualization of BLR-200 suppressed the expression of genes related to fibrosis at d21. Spatial gene expression was performed using 10× Genomics's Visium Spatial Gene Expression on formalin fixed paraffin embedded skin samples.

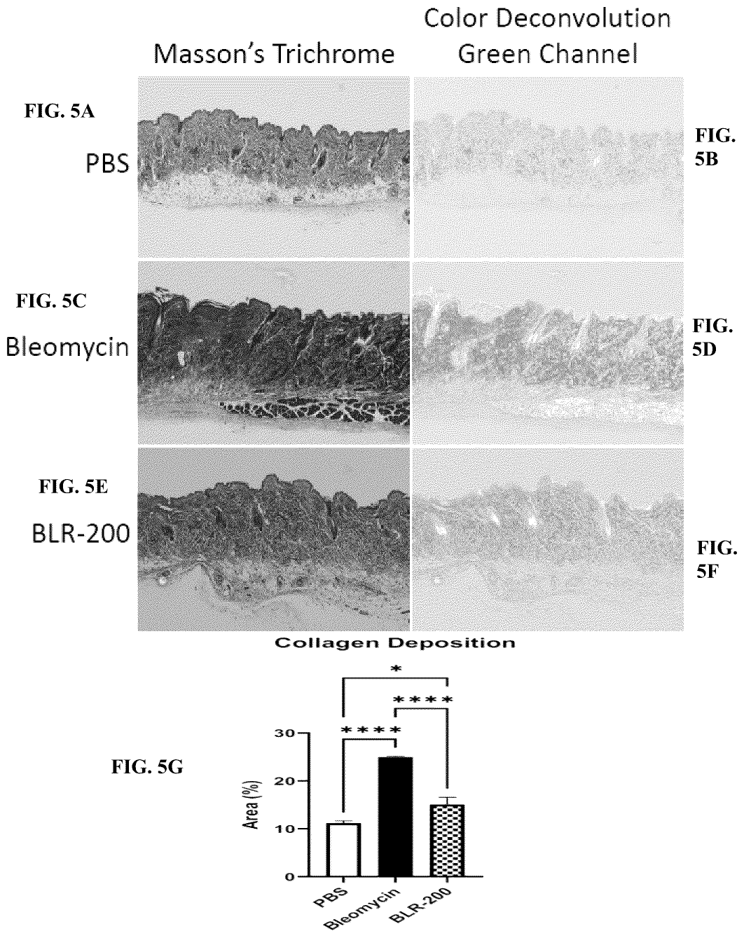

FIGS. 5A-5F show the treatment with BLR-200 at d14 post bleomycin injection partially reversed excessive collagen deposition at d28. Collagen deposition analysis was performed using color deconvolution method in ImageJ. FIG. 5J shows the data was presented as mean SEM, n=4. Statistical analysis was performed using one-way ANOVA followed by Tukey-Kramer test. *p≤0.05, *****p≤0.0001.

TABLE 2
Proteomic Analysis - Treatment/Reversal Experiment

| | Fold | |
|---|---|---|
| REACTOME Pathway | Enrichment | Genes |
| Formation of the cornified | 23.29 | DSP, CDSN, KRT2, KRT79, KRT5, |
| envelop | | DSG1A, STFA3, TGM1, KRT19, KRT17, |
| | | KRT16, KRT14, DSC1, RPTN, KRT6A |
| Integrin signaling | 18.40 | FGB, FGA, FGG, FN1 |
| MAP2K and MAPK activation | 12.74 | FGB, FGA, FGG, FN1 |
| Keratinization | 12.68 | DSP, CDSN, KRT2, KRT79, KRT5, |
| | | DSG1A, STFA3, TGM1, KRT19, KRT17, |
| | | KRT16, KRT14, DSC1, RPTN, KRT6A |
| Integrin cell surface | 9.41 | FGB, FGA, VTN, FGG, FN1 |
| interactions | | |
| Toll-like receptor cascades | 5.36 | FGB, FGA, CTSL, FGG, S100A9, |
| | | S100A8 |
| Innate immune system | 3.47 | C1QB, C1QA, CPB2, LYZ2, CFI, CFP, |
| | | C3, VTN, CTSL, BPIFA2, QSOX1, |
| | | CTSD, CAMP, DSP, FGB, FGA, ARG1, |
| | | FGG, GGH, DSG1A, MASP2, CALM4, |
| | | DSC1, S100A9, S100A8, LTF |
| Immune system | 2.23 | C1QB, C1QA, CPB2, LYZ2, CFI, |
| | | KEAP1, CFP, C3, VTN, CTSL, BPIFA2, |
| | | QSOX1, CTSD, CAMP, DSP, FGB, FGA, |
| | | ARG1, FGG, GGH, DSG1A, MASP2, |
| | | BLMH, CALM4, DSC1, S100A9, |
| | | S100A8, LTF |

Conclusion

Histology of the skin samples shows indistinguishable phenotype between BLR-200 treatment and control. Gene expression analyses show that BLR-200 prevents the expression of fibrosis-related genes induced by bleomycin. The results evidences that BLR-200 blocks bleomycin-induced fibrosis via suppressing collagen stability and the competency and commitment of progenitor cells to fibroblasts. BLR-200 demonstrates its ability to block and reverse bleomycin-induced fibrosis. The method of screening for drugs or diagnosing fibrosis will measure the gene expression

### Example 2: Therapeutic Peptides Based on CCN3 Prevent Fibrosis in a Mouse Model of Systemic Sclerosis

BLR-200 represents a novel therapeutic approach for treatment of dermal fibrosis. However, the effects of CCN3, or CCN3 derivatives, on dermal fibrosis have yet to be studied. This example uses the bleomycin-induced model of SSc dermal fibrosis, the anti-fibrotic potential of the CCN3-based peptide BLR-200 is investigated.

Methods

Bleomycin-Induced Model of Dermal Fibrosis

Bleomycin sulfate (0.1 units/100 ml per injection; Sigma) or vehicle (PBS, 100 ml per injection) was injected subcutaneously into a single location on the flank of wild-type C57BL/6J (Jackson Laboratories) mice once daily for 28 days. Bleomycin-treated mice were further divided into two treatment groups, which were injected intraperitoneally 3 times per week with either scrambled peptide (10 μg/kg) or BLR-200 (10 μg/kg). At the end of the treatment period, mice were sacrificed via CO₂ inhalation, and skin and RNA samples were collected for analysis. All animal protocols were approved by the Animal Care and Veterinary Services at Western University.

Histological Assessment of Skin Thickness

Skin samples were fixed in a 4% paraformaldehyde (Sigma) solution overnight at 4° C., and were subsequently processed, and embedded in paraffin wax. The embedded samples were then sectioned (5p m), using a Leica microtome, and collected on Superfrost Plus slides (Thermo Fisher). Skin sections were deparaffinized using Xylenes (Sigma) and rehydrated by successive immersion in descending concentrations of alcohol. Sections were then stained with haematoxylin (Sigma) for 1 minute, rinsed in water, and counterstained with eosin-y (Thermo-Fisher) for 7 minutes. Sections were visualized on a Zeiss Imager M2m microscope (Carl Zeiss, Jena, Germany). For each section, 4-6 images were taken at random. Two different depths were analyzed for each mouse. Dermal thickness of the sections was measured using Northern Eclipse software. Measurements were analyzed by one-way ANOVA with Tukey's post hoc test (p<0.05).

Histological Collagen Analysis

Skin samples were fixed, processed, embedded, and sectioned as described above. Skin sections were deparaffinized using Xylenes (Sigma) and rehydrated by successive immersion in descending concentrations of alcohol. The sections were then subjected to Masson's trichrome stain, as previously described (Quesnel et al., 2019). Sections were visualized on a Zeiss Imager M2m microscope. For each section, 4-6 images were taken at random. Collagen deposition and density were analyzed using ImageJ by measuring the percent area of the dermis stained with alanine blue. Measurements were analyzed by one-way ANOVA with Tukey's post-hoc test (p<0.05).

Skin sections were also stained with picrosirius red to further analyze collagen content. Briefly, skin sections were subjected to incubation in a solution of 0.1% Sirius red (Sigma) in saturated picric acid (Thermo Fisher) for 60 minutes, followed by two washes with 0.5% acetic acid (Sigma). Sections were visualized under circularly polarized light to assess collagen fibrillar hue through collagen birefringence properties. Images were taken at a constant light intensity, a fixed 450 angle to the polarizing filter, and the same analyzer was used to facilitate consistent comparisons. For each section, 4-6 images were taken at random. Collagen viewed using this method will appear different colors depending on fiber thickness and spatial orientation, with the color changing from blue to yellow to orange to red as fiber density increases. These properties were used to determine the proportion of different collagen colors within the dermis, a method which has been used to quantitatively analyze collagen content (Rich and Whittaker, 2005). The relative color content of the images can be obtained using ImageJ to separate the digital images into their hue, saturation, and value components, as previously described (Armstrong, 2019). Using this method, the relative amount of red, orange, yellow, and blue pixels within a given range can be expressed as a proportion of the total number of pixels representing collagen. The measurements were analyzed using a one-way ANOVA with Tukey's post hoc test (p<0.05).

3,3′-Diaminobenzidine (DAB) Immunostaining

Skin samples were fixed, processed, embedded, and sectioned as described above. Skin sections were deparaffinized using Xylenes (Sigma) and rehydrated by successive immersion in descending concentrations of alcohol. Sections were then stained using the Vectastain ABC Kit (Vector Laboratories). In brief, skin sections were subjected to sodium-citrate antigen retrieval for 30 minutes at 98° C. Non-specific binding was blocked by incubating slides with diluted 2.5% normal goat serum. Sections were then incubated with primary antibodies against αSMA (1:400; Abcam, ab5694) and YAP/TAZ (1:200; Cell Signaling, D24E4). This was followed by incubation with biotinylated secondary antibody, and then Vectastain ABC Reagent (Avidin DH, Biotinylated Horseradish Peroxidase H). Primary antibody binding was then visualized by incubation with ImmPact DAB peroxidase substrate (Vector Laboratories) and sections were counterstained with hematoxylin. Sections were visualized on a Zeiss Imager M2m using Zen Pro software. For each section, 4-6 images were taken at random and the proportion of positively stained cells per field of view were measured using ImageJ. Measurements were analyzed by one-way ANOVA with Tukey's post-hoc test (p<0.05).

Immunofluorescence

Skin samples were fixed, processed, embedded, and sectioned as described above. Skin sections were deparaffinized using Xylenes (Sigma) and rehydrated by successive immersion in descending concentrations of alcohol. The sections were then subjected to sodium-citrate antigen retrieval for 30 minutes at 98° C., followed by blocking in a solution of 10% normal serum, 0.1% Triton X-100 (Sigma) and PBS for 1 hour. Slides were then incubated with primary antibody diluted in blocking solution overnight at 4° C. The primary antibodies used were: anti-CCN1 (1:200; Millipore, ABC102), anti-CCN2 (1:100; Santa Cruz, Sc-365970), anti-CCN3 (1:1000, antibody used as described by Kyurkchiev et al., 2004), and anti-Sox2 (1:200; Santa Cruz, Sc-365823). After incubation with primary antibody, sections were rinsed and incubated with the appropriate secondary antibody conjugated to Alexa Fluor 647 (Thermo Fisher) for 1 hour. Sections were then counterstained with 4′,6-diamidino-2-phenylindole (DAPI; Thermo Fisher) and imaged on a Zeiss fluorescence microscope using Northern Eclipse software. For each section, 4-6 images were taken at random and the amount of positively stained cells in the dermis were assessed.

Quantitative Polymerase Chain Reaction (qPCR) Analysis

To collect RNA for qPCR analysis, skin tissue samples were first subjected to bead homogenization using a BeadBug homogenizer (Sigma). RNA was then purified from the samples using TRIzol reagent (Thermo Fisher) to solubilize biological material, followed by phenol-chloroform phase separation. The aqueous phase containing RNA was collected from the samples, and RNA was precipitated using isopropanol and collected by centrifugation at 12,000 rpm for 15 minutes at 4° C. The RNA pellet was then washed three times using 90% ethanol before resuspension in RNase-free water (Qiagen). RNA concentration and integrity were measured via Nanodrop and 1 μg of RNA was reverse transcribed using qScript cDNA SuperMix (Quantabio), producing a cDNA library. SYBR green real-time PCR was then performed by combining cDNA (7 ng/well), SYBR master mix (Thermo Fisher) and gene specific primers. Signal changes were detected using a ViiA7 Real-Time PCR System. The following gene-specific primers used: Acta2, Ccn1, Ccn2, Ccn3, Itga11, Sox2, Yap1, Plod2, and Lox. Samples were run in triplicate and expression values were standardized to control values from Rn18s primers using the ΔΔCt method. Statistical analysis was performed using one-way ANOVA with Tukey's post hoc test (p<0.05).

TABLE 2
Primers used for qPCR.

| | Forward | Reverse |
|---|---|---|
| Target | (5′ to 3′) | (5′ to 3′) |
| Rn18s | SEQ ID NO: 1 | SEQ ID NO: 2 |
| | GTAACCCGTTGA | CCATCCAATCGG |
| | ACCCATT | TAGTAGCG |
| Acta2 | SEQ ID NO: 3 | SEQ ID NO: 4 |
| | CATCCGACACTG | AGGTCTCAAACA |
| | CTGACA | TAATCTGGGTCA |
| Ccn1 | SEQ ID NO: 5 | SEQ ID NO: 6 |
| | TCTGCGCTAAAC | GCAGATCCCTTT |
| | AACTCAACGA | CAGAGCGG |
| Ccn2 | SEQ ID NO: 7 | SEQ ID NO: 8 |
| | TGCTGTGCATCC | CAGAGAGCGAGG |
| | TCCTACCG | AGCACCAA |
| Ccn3 | SEQ ID NO: 9 | SEQ ID NO: 10 |
| | GCGGGGAGAGTT | GTCTCCCTCTGG |
| | GTTCTGAG | AACCATGC |
| Itga11 | SEQ ID NO: 11 | SEQ ID NO: 12 |
| | CGCTGTGAATCA | CCCACAGGGCTA |
| | GACGAGGT | TTCTTGT |
| Sox2 | SEQ ID NO: 13 | SEQ ID NO: 14 |
| | CTCCGCAGCGAA | AGTCGGCATCAC |
| | ACGACAG | GGTTTTTG |
| Yap1 | SEQ ID NO: 15 | SEQ ID NO: 16 |
| | GAACTCGGCTTC | AGGGTCAAGCCT |
| | AGGTCCTC | TGGGTCTA |
| Plod2 | SEQ ID NO: 17 | SEQ ID NO: 18 |
| | CAGGAACATGGG | GACGTGTCACAA |
| | CATGGATTTC | GAGGAGCAA |
| Lox | SEQ ID NO: 19 | SEQ ID NO: 20 |
| | AAATGGGTATCC | GTGCGTTAGAGG |
| | AACAAATGGC | ACAACAGGA |

Proteomic Analysis

Proteomic analysis was carried out at the Proteomic Resource Facility (PRF) in the Department of Pathology at the University of Michigan, where mass spectrometry-based Tandem Mass Tag (TMT, Thermo Fisher) was employed. Full skin protein samples were homogenized and digested with trypsin. Protein fragments were individually labeled with one of ten isobaric mass tags following the manufacturer's protocol. After labelling, equal amounts of peptide from each condition were mixed. The labelled proteins were then fractionated by 2D-liquid chromatography, using basic pH reverse-phase separation followed by acidic pH reverse-phase. The samples were analyzed on a high-resolution, tribrid mass spectrometer (Orbitrap Fusion Tribrid, Thermo Fisher) using conditions optimized by the PRF. MultiNotch MS3 approach was employed to obtain accurate quantitation of the identified proteins. Data analysis was performed using Proteome Discoverer (v 2.3, Thermo Fisher). MS2 spectra were searched against the SwissProt reviewed mouse protein database (downloaded on 2019 Jun. 29) using the following search parameters: MS1 and MS2 tolerance were set to 10 ppm and 0.6 Da, respectively; carbamidomethylation of cysteines (57.02146 Da) and TMT labeling of lysine and N-termini of peptides (229.16293 Da) were considered static modifications; oxidation of methionine (15.9949 Da) and deamidation of asparagine and glutamine (0.98401 Da) were considered variable. Identified proteins and peptides were filtered to retain only those that passed ≤2% false-discovery rate (FDR) threshold of detection. Quantitation was performed using reporter ion intensity extracted from high-quality MS3 spectra within a ±10 PPM window centered on the theoretical m/z value of each reporter ion. Reporter ion intensities were corrected for isotopic impurities of different TMT reagents as specified by the manufacturer. Only those peptide reporter ion intensities with an average signal-to-noise ratio of 9 and <40% co-isolation interference were considered for quantification. Differential protein expression between conditions, normalizing to control (PBS) for each subject's specimens separately was established using edgeR (Robinson et al., 2010). Then, results for individual proteins from six mice per treatment were pooled. Fold change ratios were produced for either bleomycin+scrambled mice or bleomycin+BLR-200 mice, relative to PBS. Differentially expressed proteins were filtered based on a ±1.8-fold cut-off (p<0.05). Reactome V69 (reactome.org) was used for pathway enrichment analyses.

Results

BLR-200 Prevents Bleomycin-Induced Changes in Skin Thickness and Collagen Organization

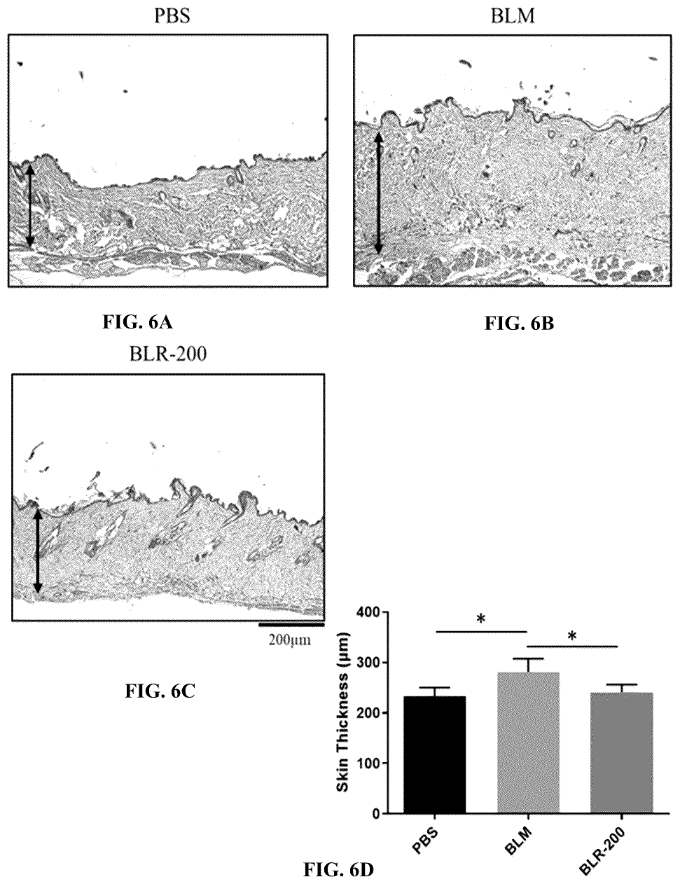

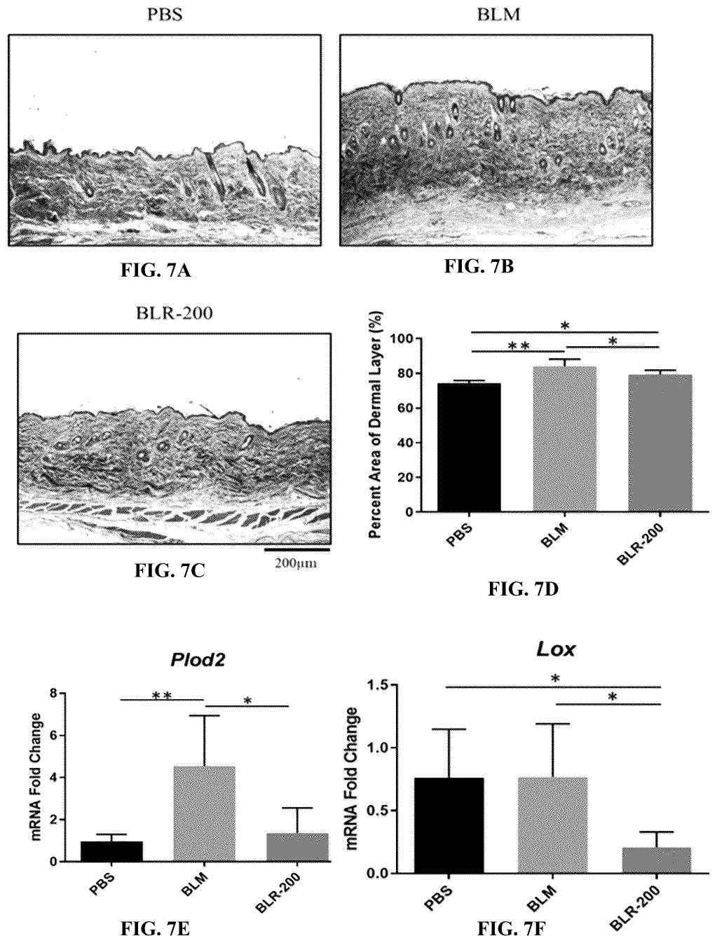

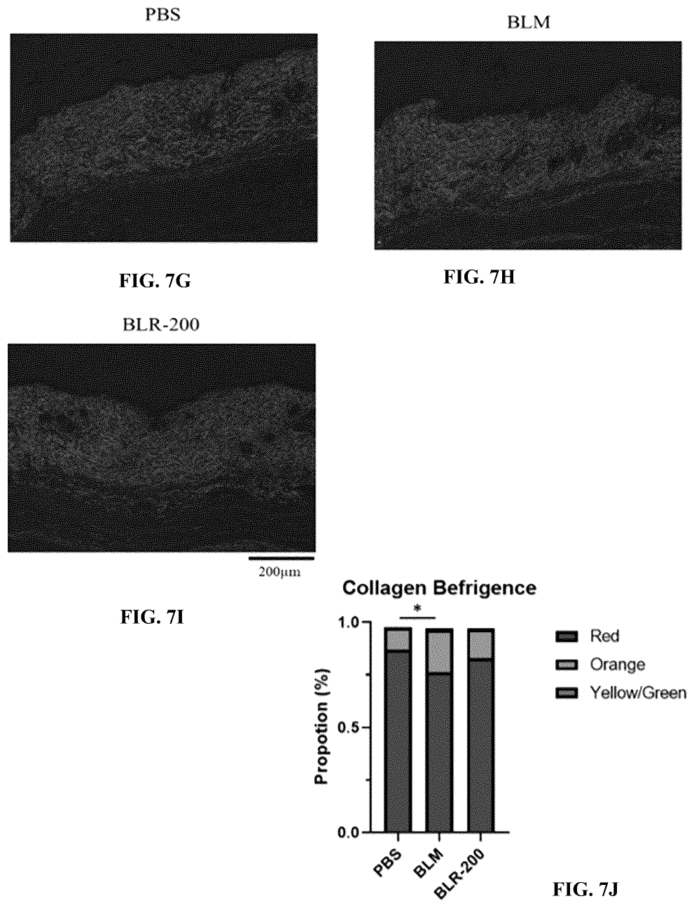

To begin to assess the effect of BLR-200 on bleomycin-induced dermal fibrosis, skin sections were histologically examined using H&E and trichrome staining. Skin thickness and collagen deposition are commonly the most observable fibrotic changes that occur in bleomycin-induced dermal fibrosis (Liu et al., 2011; Quesnel et al., 2019). In the scrambled peptide control group (BLM; bleomycin), bleomycin injections caused an increase in dermal thickness; a change that was impaired in mice receiving BLR-200 treatment (BLR-200) (FIG. 6A-6D). BLM mice also had increased collagen deposition in the dermis and exhibited consistently disorganized collagen organization (FIG. 7A-7D). These changes in collagen deposition and organization were diminished in BLR-200 mice.

To further assess fibrotic changes in collagen, qPCR analysis was employed to assess the mRNA expression of Plod2 and Lox, genes that encode proteins that promote collagen crosslinking. PLOD2 hydroxylates lysine residues of collagen telopeptides, thus increasing the amount of pyridinoline cross-links, which promote irreversible accumulation of collagen in fibrotic tissue (Gjaltema et al., 2015; van der Slot et al., 2003). LOX facilitates the accumulation of stable collagen by increasing the amount of covalent cross-linking in collagen strands (Chen et al., 2018). Both PLOD2 and LOX protein expression are increased in fibroblasts isolated from lesional areas of SSc patients (Meyringer et al., 2007; Nguyen et al., 2021; van der Slot et al., 2003). In this study, BLR-200 treatment prevented the bleomycin-induced increase in Plod2 and Lox mRNA expression (FIG. 7E-7F).

Finally, picrosirius red staining was used, in combination with circularly polarized light, as a highly sensitive means to visualize collagen fibers. Fibrillar hue was used to assess structural changes in collagen in mice treated with bleomycin+/−scrambled peptide (BLM) or BLR-200 (BLR-200). BLM mice contained a significantly higher proportion of yellow/orange-stained collagen, representing newly synthesized collagen (FIG. 7G-7J) (Armstrong, 2019). BLR-200 impaired this bleomycin-induced increase in newly synthesized collagen. Overall, these results suggest that BLR-200 treatment impairs general fibrotic changes associated with bleomycin-induced dermal fibrosis.

FIGS. 6A-6D. BLR-200 prevents the bleomycin-induced increase in skin thickness.

Bleomycin sulfate (0.1 units/100 ml per injection) or PBS (100 ml per injection) was injected subcutaneously into a single location on the flank of wild-type C57BL/6J mice once daily for 28 days. Bleomycin-treated mice were further divided into two treatment groups, which were injected intraperitoneally 3 times per week with either 10 μg/kg scrambled peptide (BLM) or 10 μg/kg BLR-200 (BLR-200). At the conclusion of the experiment, dermal tissue was fixed and stained with H&E. Representative images are shown. Dermal thickness measurements were taken at 3 different points along 2 depths of skin, averaged for each mouse, and compared among treatment groups (one-way ANOVA with Tukey's post hoc test; *p<0.005).

FIGS. 7A-7D show that BLR-200 prevents bleomycin-induced collagen changes in the dermis. Wild-type C57BL/6J mice were either treated with PBS (n=6), bleomycin+scrambled peptide (BLM; n=6), or bleomycin+BLR-200 (BLR-200; n=6) (FIGS. 7A-7D) Dermal tissue was stained with Masson's trichrome stain to examine histological changes in collagen organization. Images were taken at 3 different points along 2 different depths of skin. Representative images are shown. The percent area of the dermal layer stained for trichrome was determined using ImageJ (one-way ANOVA with Tukey's post hoc test; **p<0.002; *p<0.05). (FIGS. 7E-7F) Total RNA was obtained from skin and subjected to SYBR Green real-time PCR. Primers were used to detect collagen cross-linking genes Plod2 and Lox. Relative gene expression, compared to the housekeeping gene Rn18s, was determined using the ΔΔCt method (one-way ANOVA and Tukey's post-host test; **p<0.002; *p<0.05). (FIGS. 7G-7J) Skin sections were stained with picrosirius red and collagen birefringence properties were analyzed to investigate newly synthesized collagen and fiber density. A one-way ANOVA and Tukey's post-host test were used to analyze the data (*p<0.05).

BLR-200 Treatment Prevents the Bleomycin-Induced Increase in αSMA-Positive Myofibroblasts

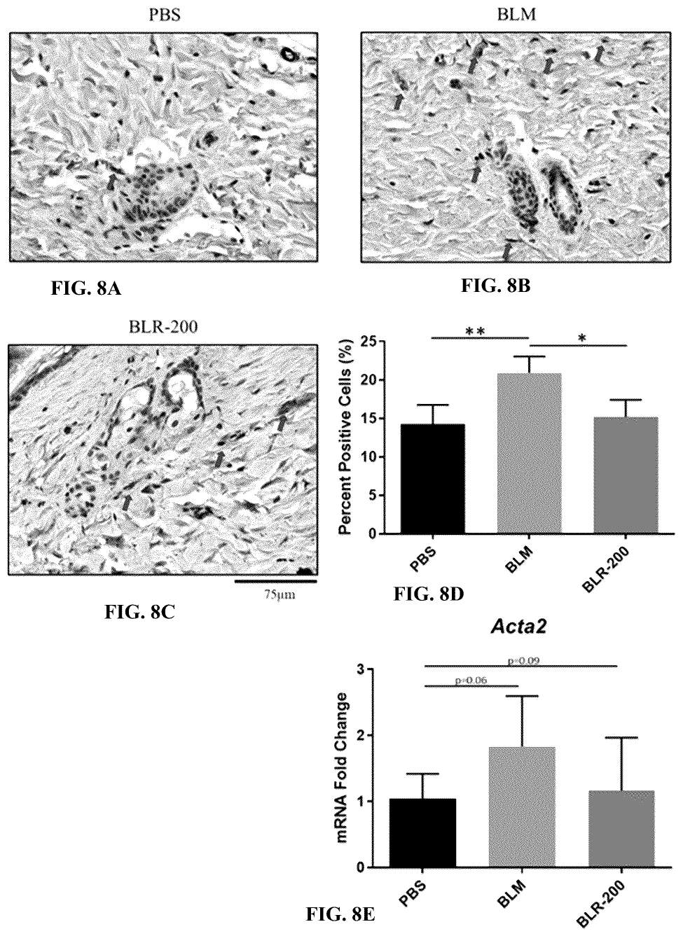

A hallmark of progressive fibrosis is the presence of activated myofibroblasts characterized by the expression of the highly contractile αSMA (Gabbiani, 2003). In order to assess the amount of myofibroblasts expressing αSMA, skin sections from the fibrotic lesion were examined using DAB immunostaining with an anti-αSMA antibody. Compared to PBS-injected control mice, mice subcutaneously injected with bleomycin showed, in the presence of scrambled peptide, a significant increase in the amount of αSMA-expressing myofibroblasts in the dermis. This increase in number of αSMA-expressing myofibroblasts in the presence of bleomycin was significantly decreased in the presence of BLR-200 (FIG. 8A-8D). Finally, RNA was extracted from whole skin and subjected to SYBR Green qPCR with primers detecting Acta2 (murine gene encoding αSMA) and showed similar trends (FIG. 8E). These results indicate that BLR-200 treatment is able to prevent activation of highly contractile myofibroblasts in the fibrotic lesion.

FIGS. 8A-8E. BLR-200 impairs bleomycin-induced increase in αSMA-expressing myofibroblasts. Wildtype C57BL/6J mice were either treated with PBS (n=6), bleomycin+scrambled peptide (BLM; n=6), or bleomycin+BLR-200 (BLR-200; n=6). (FIGS. 8A-8D) Fixed dermal tissue sections were incubated with an anti-αSMA antibody and antibody localization was visualized using DAB chromogen. Representative images are shown. The percentage of fibroblasts positive for αSMA was determined using ImageJ (one-way ANOVA and Tukey's post hoc test; **p<0.002; *p<0.05). (FIG. 8E) Total RNA was extracted from the skin and subjected to SYBR Green real-time PCR using primers detecting Acta2 (murine gene encoding αSMA). Relative Acta2 expression, compared to the housekeeping gene Rn18s, was determined using the ΔΔCt method (one-way ANOVA and Tukey's post-hoc test; *p<0.05).

Proteomic Analysis Reveals that BLR-200 Prevents the Bleomycin-Induced Increase in ECM-Related Proteins

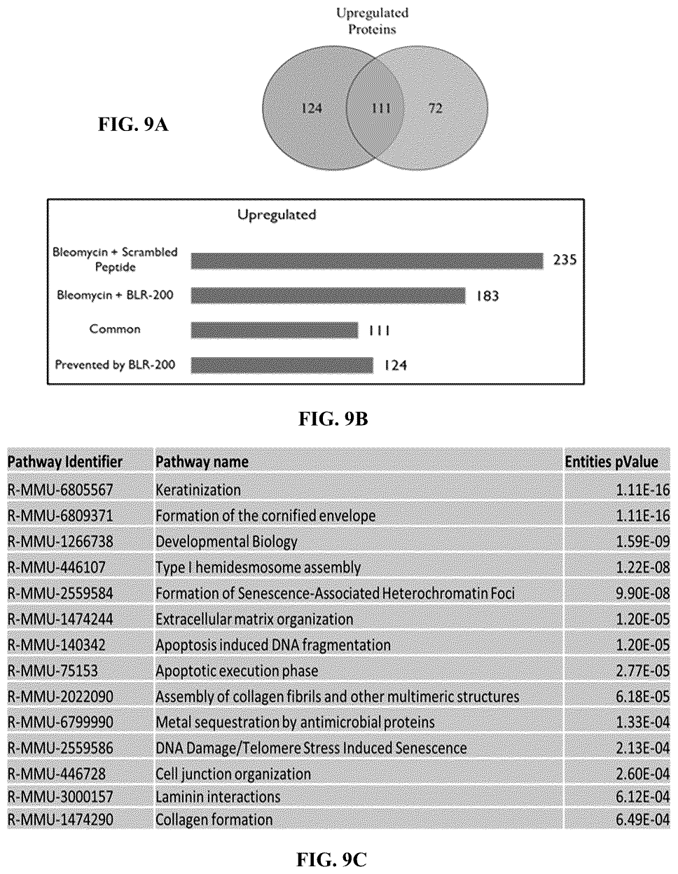

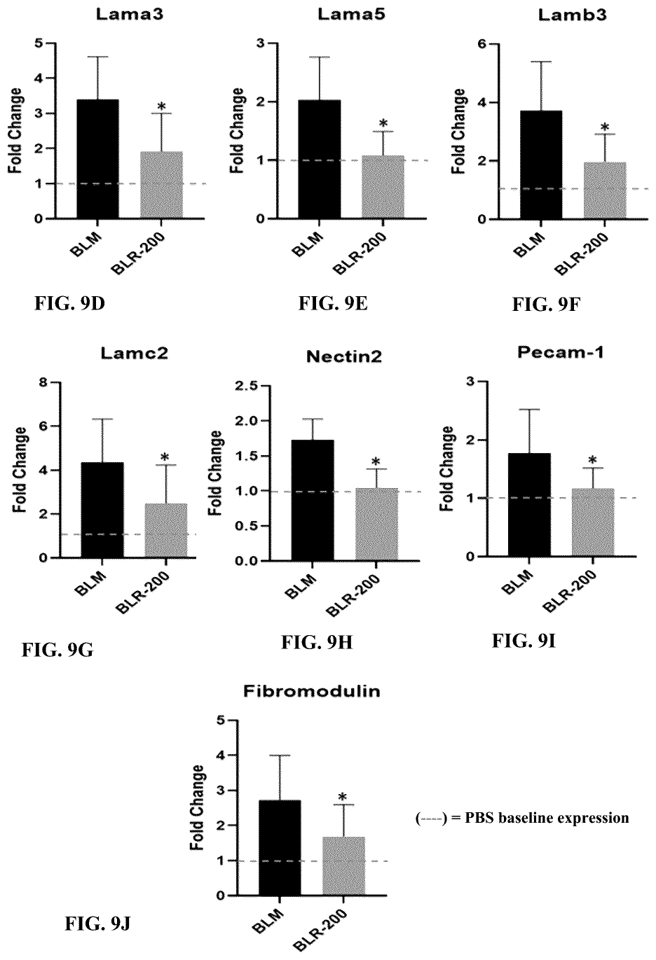

To further assess the effect of BLR-200 on bleomycin-induced dermal fibrosis, an unbiased proteomic approach was employed. Protein was extracted from full skin and subjected to tandem mass-tag mass spectrometry. Protein expression for BLM mice and BLR-200 mice was normalized to protein expression in the control PBS mice, producing fold change differences. A list of upregulated proteins in each experimental group was generated using a 1.8-fold cut-off (p<0.05). Out of the upregulated proteins in BLM mice, 124/235 were prevented by BLR-200 treatment (FIG. 9A-9B). Proteins that were induced in BLM mice, but not BLR-200 mice were analyzed using the Reactome pathway database. It was found that several ECM-related pathways were prevented by BLR-200 treatment, including extracellular matrix organization (R-MMU-1474244), assembly of collagen fibrils and other multimeric structures (R-MMU-2022090), laminin interactions (R-MMU-3000157) and collagen formation (R-MMU-1474290) (FIG. 9C). Further analysis revealed that BLR-200 treatment specifically prevents induction of several ECM-associated proteins, including laminin (lam)a5, lama3, lamb3, lamc2, nectin-2, pecam-1, and fibromodulin (FIG. 9D). Laminins, glycoproteins in the extracellular matrix, promote cell adhesion and migration; they may also play a role in fibroblast proliferation (Domogatskaya et al., 2012; Liu et al., 2020). Fibromodulin, a proteoglycan that plays a role in collagen fibril assembly (Kalamajski et al., 2016), has been linked to cardiac remodeling and liver fibrosis (Andenos et al., 2018; Mormone et al., 2012). Nectin-2 and Pecam-1, cell adhesion molecules, enhance inflammation, cell adhesion, and cell migration (Woodfin et al., 2007; Yamada et al., 2017). Collectively, these results provide further evidence that BLR-200 prevents bleomycin-induced skin fibrosis.

FIGS. 9A-9J. BLR-200 prevents changes in protein expression patterns associated with bleomycin-induced dermal fibrosis.

Wildtype C57BL/6J mice were either treated with PBS (n=6), bleomycin+scrambled peptide (BLM; n=6), or bleomycin+BLR-200 (BLR-200; n=6). Total protein was extracted from the skin and subjected to tandem mass-tag mass spectrometry. Relative protein expression for BLM mice and BLR-200 mice was normalized to the control PBS group, generating fold change differences for all detected proteins. A list of upregulated proteins in each experimental group was generated using a 1.8-fold cut-off (p<0.05). (FIGS. 9A-9B) Venn diagram showing that 124 proteins that were induced in BLM mice are prevented by BLR-200 treatment. (FIG. 9C) Reactome pathway analysis of bleomycin-induced proteins that were prevented by BLR-200 treatment shows that the many of the top pathways prevented include ECM-associated processes, including keratinization, extracellular matrix organization, collagen formation, and laminin interactions. (FIGS. 9D-9J) Examining fold change differences of individual proteins revealed that specific laminins (a3, a5, b3, c2) and fibrosis-related proteins (nectin-2, pecam-1, fibromodulin) were prevented by BLR-200 treatment. A student's t-test was used to analyze differences in fold change (*p<0.05).

BLR-200 Prevents the Bleomycin-Induced Increase in CCN1 and CCN2 Expression

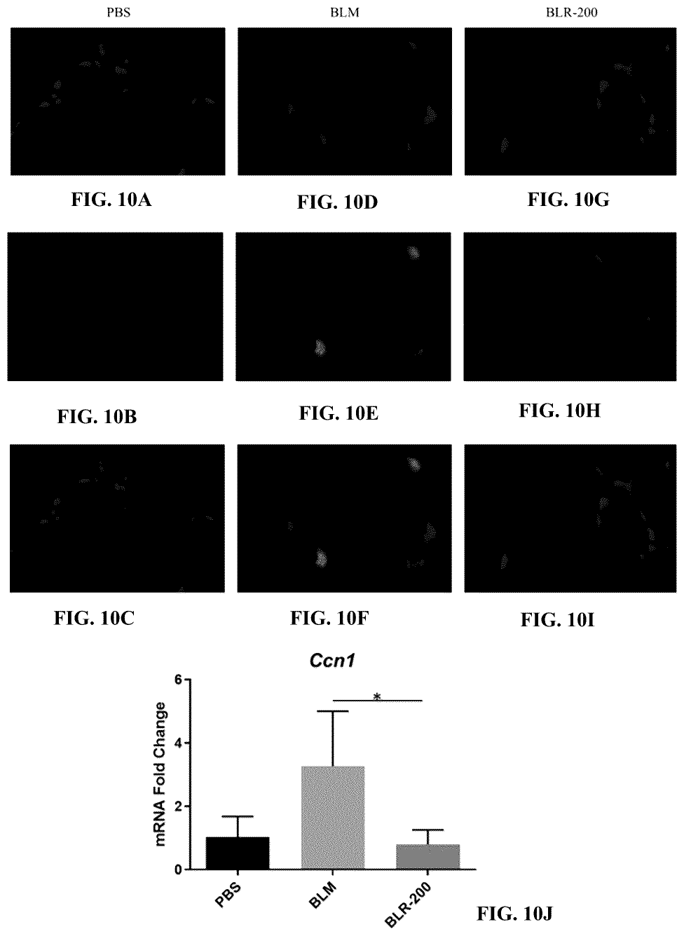

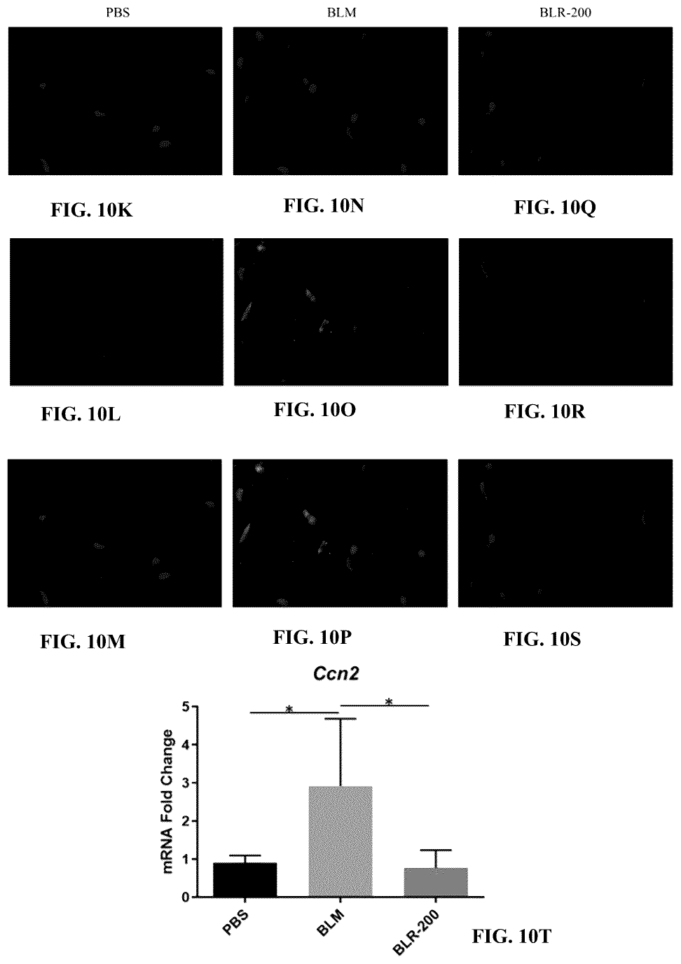

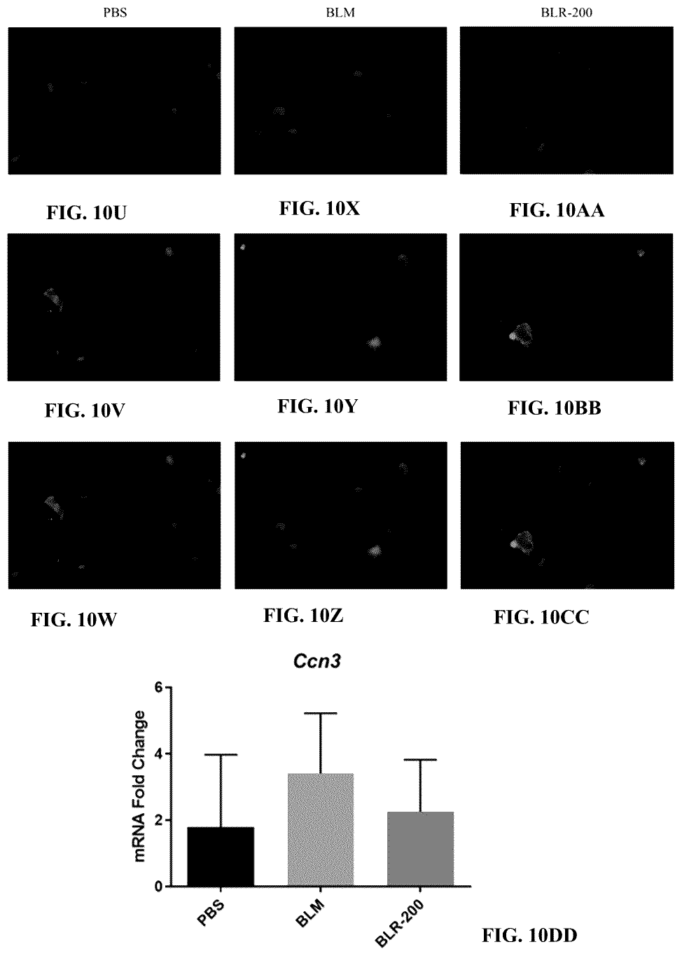

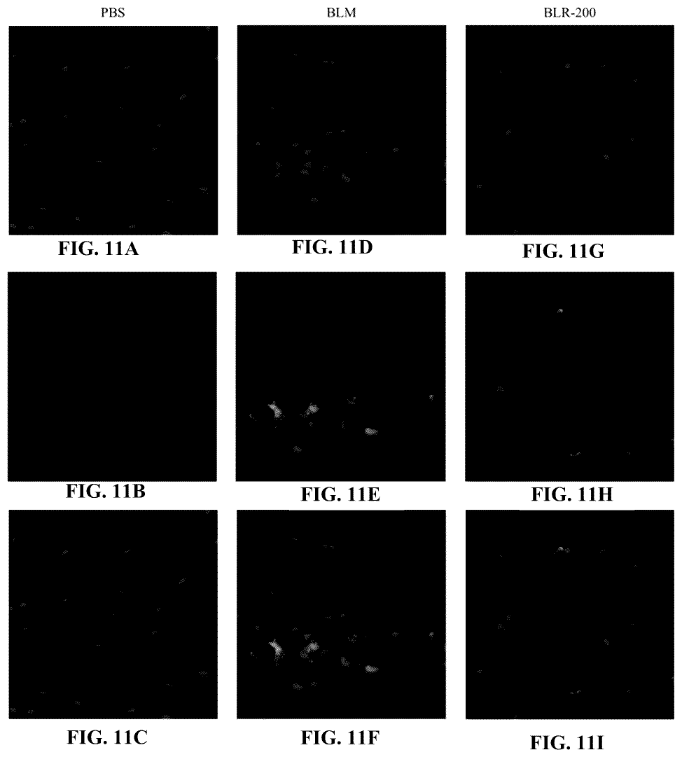

Both CCN1 and CCN2 are markers and mediators of bleomycin-induced skin fibrosis (Liu et al., 2011; Quesnel et al., 2019). Furthermore, CCN1 and CCN2 are reciprocally regulated to CCN3 in response to TGFβ1 in dermal fibroblasts. Moreover, ample evidence suggests that CCN3 may have inhibitory activity on CCN1 and CCN2 (Riser et al., 2014). Based on these observations, the CCN3-based peptide, BLR-200, would suppress the bleomycin-induced increases in CCN1 and CCN2 protein and mRNA expression. To test this notion, immunohistochemistry was used to detect protein localization within the dermis, and qPCR, to detect mRNA expression. As anticipated the detected protein levels of both CCN1 and CCN2 were significantly increased in the dermis of BLM mice; however, these increases were not observed in BLR-200 treated mice (FIG. 10A-10J and FIG. 10K-10T). Similar results were observed when gene expression of Ccn1 and Ccn2 was detected. Conversely, no significant alterations in CCN3 protein localization or Ccn3 gene expression were observed with any treatment (FIGS. 10U-10DD).

FIGS. 10A-10DD. BLR-200 treatment prevents bleomycin-induced changes in CCN1 and CCN2. Wildtype C57BL/6J mice were either treated with PBS (n=6), bleomycin+scrambled peptide (BLM; n=6), or bleomycin+BLR-200 (BLR-200; n=6). Dermal tissue was fixed, stained and visualized by immunohistochemistry with primary antibodies against CCN1, CCN2, and CCN3. Representative images are shown. Total RNA was also extracted from the skin and subjected to SYBR Green real-time PCR using primers detecting Ccn1, Ccn2 and Ccn3. Relative mRNA expression, compared to the housekeeping gene Rn18s, was determined using the ΔΔCt method (one-way ANOVA and Tukey's post-hoc test; *p<0.05). BLR-200 prevents bleomycin-induced increase in (FIGS. 10A-10J) CCN1 and (FIGS. 10K-10T) CCN2 protein and mRNA expression. (FIGS. 10U-10DD) No significant changes were observed with CCN3 expression.

BLR-200 Prevents the Bleomycin-Induced Increase of the Plasticity Marker SOX2 in the Reticular Dermis

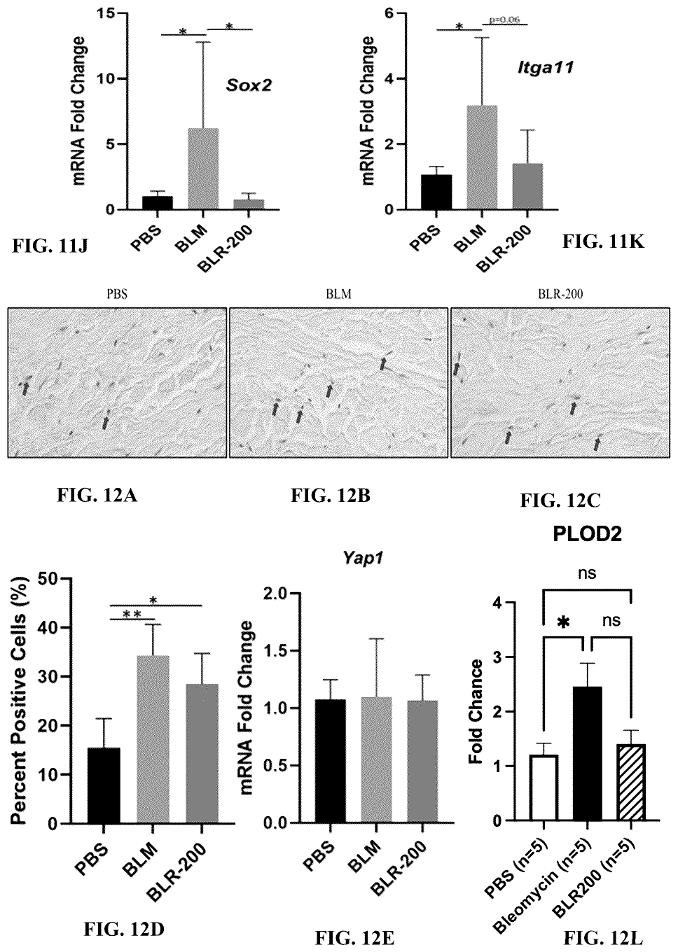

The origin of pro-fibrotic αSMA-expressing myofibroblasts is unclear, although emerging evidence suggests these cells are derived from plastic dermal fibroblasts via a progenitor cell-like intermediate (Chadli et al., 2019; Tsang et al., 2019). In bleomycin-induced skin fibrosis, a majority of myofibroblasts stain positive for the progenitor/plasticity marker SOX2 (Liu et al., 2014). Furthermore, CCN2 expression by SOX2-expressing cells has been shown to contribute to recruitment of these progenitor cells to the fibrotic lesion in bleomycin-induced fibrosis (Tsang & Leask, 2014). Tested was the hypothesis that BLR-200 could reduce the appearance/recruitment of SOX2-positive cells to the dermis. To test this notion, skin sections were stained with an anti-SOX2 antibody. SOX2-positive cells were significantly increased in the reticular dermis of BLM mice, but not in BLR-200 mice (FIG. 11A-11I). In models of melanoma metastasis, a subset of αSMA-expressing myofibroblasts (myoCAFs) express high levels of both SOX2 and al 1 integrin; this subtype of myoCAFs negatively correlates with disease-free survival (Tsang et al., 2019; Zeltz et al., 2019). Moreover, α11 integrin expression contributes to myofibroblast differentiation in multiple fibroblast types in vitro (Talior-Volodarsky et al., 2012; Tsang et al., 2019). Accordingly, whole skin tissue samples were also subjected to qPCR to detect Sox2 and Itga11 mRNA expression. Both Sox2 and Itga11 expression were increased in BLM mice; these increases were prevented by BLR-200 treatment (FIG. 11J-11K).

FIGS. 11A-11K. BLR-200 treatment prevents expression of the plasticity marker SOX2. Wildtype C57BL/6J mice were either treated with PBS (n=6), bleomycin+scrambled peptide (BLM; n=6), or bleomycin+BLR-200 (BLR-200; n=6). (FIGS. 11A-11I) Dermal tissue was fixed, stained and visualized by immunohistochemistry with primary antibodies against SOX2. Representative images are shown. (FIGS. 11J-11K) Total RNA was extracted from the skin and subjected to real-time PCR using primers detecting Sox2 and ItgA11. Relative Sox2 and Itga11 expression, compared to the housekeeping gene Rn18s, was determined using the ΔΔCt method (one-way ANOVA and Tukey's post-hoc test; *p<0.05).

The Anti-Fibrotic Activity of BLR-200 is Likely Downstream of YAP1 Mechanotransduction Signaling

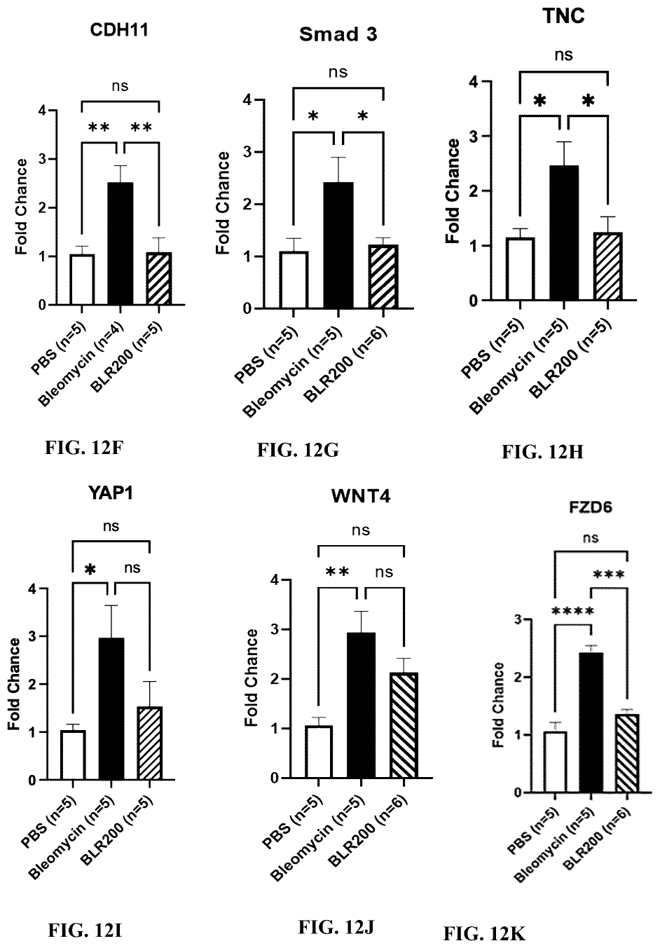

Upregulation and activation of the mechanosensitive transcriptional cofactor YAP1 promotes the perpetuation of pro-contractile myofibroblasts that are essential for fibrosis (Schulz et al., 2018; Shi-wen et al., 2021). Constitutive nuclear localization of activated YAP1 is also a feature of fibroblasts derived from the fibrotic lesions of SSc patients (Toyama et al., 2018). Since BLR-200 treatment is able to prevent multiple fibrotic changes in bleomycin-induced fibrosis, next assessed was the expression of YAP1 in the dermis of experimental mice. To assess the number of cells expressing YAP1, skin sections were examined using DAB immunostaining with an anti-YAP1 antibody. Compared to PBS-injected mice, BLM mice showed, in the presence of scrambled peptide, a significant increase in the number of YAP1-expressing cells in the dermis. Unexpectedly, the number of YAP1-expressing cells was biologically altered by BLR-200 treatment (FIG. 12A-12D). Whole skin sections were also subjected to SYBR Green qPCR analysis with primers for Yap1 and showed biological changes (FIG. 12E). Although the nuclear localization of YAP1 was not assessed, these results indicate that BLR-200 may be exhibiting its anti-fibrotic effects downstream of YAP mechanotransduction signaling.

FIGS. 12A-12E. BLR-200 treatment does biologically effect bleomycin-induced changes in YAP1 expression in the dermal layer.

Wildtype C57BL/6J mice were either treated with PBS (n=6), bleomycin+scrambled peptide (BLM; n=6), or bleomycin+BLR-200 (BLR-200; n=6). (FIGS. 12A-12D) Fixed dermal tissue sections were incubated with an anti-YAP1 antibody and antibody localization was visualized using DAB chromogen. Representative images are shown. The percentage of fibroblasts positive for YAP1 was determined using ImageJ (one-way ANOVA with Tukey's post hoc test; *p<0.002; *p<0.05). (FIG. 12E) Total RNA was extracted from the skin and subjected to SYBR Green real-time PCR using a primer for Yap1. Relative Yap1 expression, compared to the housekeeping gene Rn18s, was determined using the ΔΔCt method (one-way ANOVA and Tukey's post-hoc test; *p<0.05).

FIGS. 12F-12L are graphs showing the BLR-200 Blocks Bleomycin-induced mRNA Expression. Mice were subjected, for 21 days, to bleomycin-induced skin fibrosis in the presence or absence of BLR-200 or scrambled peptide. mRNAs were extracted and mRNA expression [cadherin 11 (FIG. 12F, CDH11), Smad3 (FIG. 12G), tenascin-C (FIG. 12H, TNC), YAP1 (FIG. 12I), Sox2 (not shown), WNT4 (FIG. 12J), frizzled 6 (FZD6, FIG. 12K) and PLOD2 (FIG. 1L) was evaluated by real-time polymerase chain (PCR), relative to b-actin control, using the DDCt method.

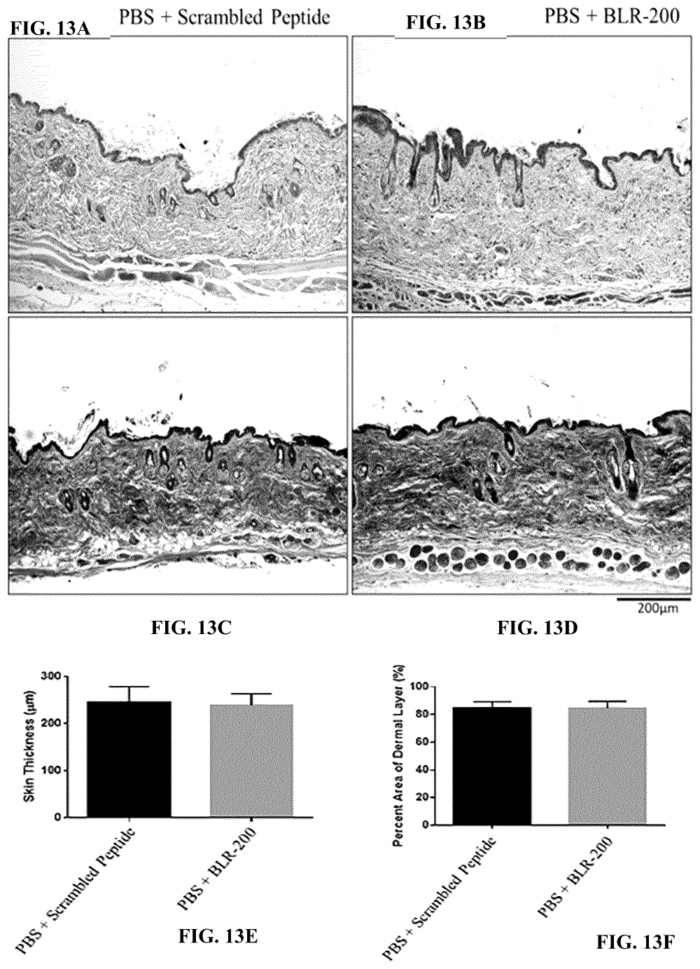

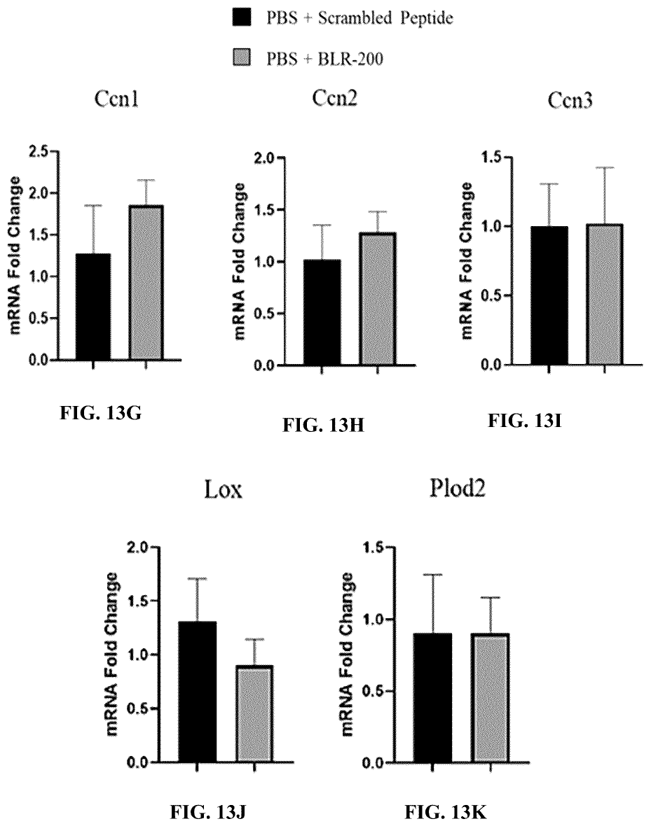

FIGS. 13A-13K, BLR-200 does not have any significant effect on normal healthy tissue.

Wild-type C57BL/6J mice were treated with PBS+scrambled peptide (n=6) or PBS+BLR-200 (n=6). (FIGS. 13A-13B) At the conclusion of the experiment, dermal tissue was fixed and stained with H&E. Representative images are shown. Dermal thickness measurements were taken at 3-6 random points in each section, averaged for each mouse, and compared (students t-test; *p<0.05). (FIGS. 13C-13D) Dermal tissue was stained with Masson's trichrome stain to examine histological changes in collagen organization. Images were taken at 3-6 random points in each section. Representative images are shown. The percent area of the dermal layer stained for trichrome was determined using ImageJ (student's t-test; *p<0.05). (FIGS. 13E-13K) Total RNA was extracted from the skin and subjected to SYBR Green real-time PCR using primers detecting Ccn1, Ccn2, Ccn3, Lox, and Plod2. Relative mRNA expression, compared to the housekeeping gene Rn18s, was determined using the ΔΔCt method (one-way ANOVA and Tukey's post-hoc test; *p<0.05).

Discussion

Currently, only limited treatment options exist for SSc patients, highlighting the need to identify novel anti-fibrotic therapies. In recent years, mechanisms underlying fibrogenesis, including in SSc, have been the target of many studies. The CCN family of matricellular proteins has emerged as an important player in the development of skin and organ fibrosis in SSc. Prominent members of this family, CCN1 and CCN2 are upregulated in the connective tissue of early onset SSc patients and likely have pro-fibrotic effects (Dziadzio et al., 2005; Gardner et al., 2006; Sonnylal et al., 2010). In contrast, CCN3 is downregulated in SSc (Xu Shiwen and Richard Stratton, unpublished data), and has anti-fibrotic effects in multiple models of fibrosis, including the kidney (Riser et al., 2014). It was shown here that targeting the regulation and activity of CCN proteins using BLR-200, a proprietary 14 amino acid peptide corresponding to an amino acid sequence within CCN3, prevents fibrosis in the bleomycin-induced model of SSc.

Mice injected subcutaneously with bleomycin exhibit vasculopathy, leading to inflammatory changes that recapitulate those which occur in the early stages of SSc (Yamamoto et al., 1999; Yamamoto & Katayama, 2011). Localized inflammation permits a fibrotic ECM, leading to activation of myofibroblasts and deposition of collagen, culminating in skin thickening due to fibrosis (King et al., 2018). A hallmark of fibrotic disease is the persistent presence of activated contractile myofibroblasts characterized by expression of αSMA (Darby et al., 1990). In this study, BLR-200 prevented the bleomycin-induced increase in skin thickness, collagen deposition and αSMA-expressing activated myofibroblasts. These are classical markers of bleomycin-induced dermal fibrosis and these data indicate that BLR-200 can prevent generalized fibrotic changes in this model. These results closely align with previous studies showing that loss of CCN2 by fibroblasts prevents bleomycin-induced fibrosis (Liu et al., 2011), supporting the idea that BLR-200 has effects mirroring CCN2 deficiency. The data also show that BLR-200 prevented the bleomycin-induced changes in collagen synthesis, organization, and mRNA expression of collagen cross-linking genes Plod2 and Lox. PLOD2 is upregulated in fibrotic conditions, including in SSc, and promotes irreversible accumulation of collagen (Nguyen et al., 2021; van der Slot et al., 2003). LOX also facilitates over-accumulation of stable collagen in fibrotic conditions (Chen et al., 2018). Stable, irreversible collagen deposition is a hallmark of fibrotic disorders and contributes to the mechanical tension and subsequent myofibroblast activation within the fibrotic microenvironment. Thus, prevention of Plod2 and Lox expression is an important finding in this study. Previous studies have also shown that CCN1 plays a pro-fibrotic role in facilitating collagen synthesis and accumulation in the skin, and loss of CCN1 has direct effects on mRNA expression of Plod2 and Lox (Quesnel et al., 2019). Collectively, these data indicate that the anti-fibrotic activity of BLR-200 operates through preventing the pro-fibrotic activity of both CCN1 and CCN2.

Providing further support of this idea, the data also indicate that BLR-200 treatment prevents the bleomycin-induced increase in Ccn1 and Ccn2 mRNA expression. Moreover, the number of cells expressing either CCN1 or CCN2 in the dermis was prevented by BLR-200 treatment. As previously mentioned, CCN3 has been proposed to be an endogenous inhibitor of CCN2 and has opposing effects to both CCN1 and CCN2 in models of diabetic nephropathy (Riser et al., 2009). Until now, the ability of CCN3 to prevent CCN1 and CCN2 expression in animal models of dermal fibrosis has not been studied. Thus, the findings provide valuable insights on the anti-fibrotic activity of CCN3. It is not known whether CCN3 has a direct inhibitory effect on the regulation of these proteins, or if this inhibition is downstream of BLR-200's effects. Further studies will be required to determine the exact mechanism of BLR-200 activity.

Unbiased proteomic analysis revealed that BLR-200 prevented the bleomycin-induced increase in expression of ECM-related proteins. It was found that bleomycin-induced fibrosis caused an increase in proteins related to ECM organization and collagen formation, including nectin-2, pecam-1, fibromodulin, and several laminins (a5, a3, b4, and c3). Treatment with BLR-200 attenuated these changes. Fibromodulin is upregulated in multiple models of fibrosis and plays a role in collagen fibril assembly, having been shown to directly interact and enhance the activity of LOX (Andenos et al., 2018; Kalamajski et al., 2016; Mormone et al., 2012). Thus, BLR-200 treatment seems to be affecting multiple facets of bleomycin-induced collagen deposition and organization. Laminins promote cell adhesion, differentiation, migration, and proliferation (Domogatskaya et al., 2012). They are upregulated in multiple fibrotic conditions including liver fibrosis and in the serum of patients with SSc (Kanaizuka et al., 1991; Santos et al., 2005). They have also been shown to facilitate the TGFβ-induced expression matrix degrading enzymes in synovial fibroblasts (Hoberg et al., 2007). Moreover, Lama5 has been shown to modulate fibroblast proliferation and adhesive signalling in epidural fibrosis (Liu et al., 2020). Collectively, these data further emphasize the ability of BLR-200 to prevent fibrosis in the bleomycin model of skin fibrosis.

Previous experiments examining the origin of myofibroblasts in the fibrotic lesions of bleomycin-induced dermal fibrosis have revealed that plastic dermal fibroblasts become induced to express the progenitor cell marker SOX2, and these cells become activated into an αSMA-expressing myofibroblast, in a CCN2-dependent manner (Liu et al., 2014; Tsang & Leask, 2014). BLR-200 treatment prevented the bleomycin-induced overexpression of Sox2 mRNA and reduced the number of SOX2-positive cells in the reticular dermis. Taken together with the finding that BLR-200 prevented the number of αSMA-expressing myofibroblasts, the data indicate that BLR-200 treatment likely impairs the CCN2-dependent activation of plastic dermal fibroblasts into myofibroblasts.

Recent evidence suggests that the persistent activated myofibroblast phenotype in fibrotic lesions occurs due to a constitutively activated, pro-adhesive and mechanotransducive autocrine signaling loop. One of the key mediators of mechanotransduction in myofibroblasts is the transcriptional cofactor YAP1. YAP1 is activated downstream of adhesive signalling and mediates the transcription of several pro-fibrotic and pro-adhesive factors, including αSMA and CCN2 (Dupont et al., 2011; Leask et al., 2003; Shi-wen et al., 2021). Increasing expression of these genes contributes to a pro-fibrotic microenvironment that further stimulates myofibroblast activation. Since YAP1 is a known mediator of this response, we investigated the effects of BLR-200 on its expression. In this first study, BLR-200 treatment did not appear to have effects on the bleomycin-induced increase in expression of YAP1 that were statistically significant. However, further data supports a biologically relevant effect of BLR-200 on YAP-1. Since BLR-200 prevents expression of downstream targets of YAP1, including Ccn1 and Ccn2, alterations in YAP1 expression. The anti-fibrotic effects of BLR-200 could also occur downstream of YAP1 signalling. Activated YAP1 translocates to the nucleus, and constitutive nuclear localization of YAP1 is a feature of fibroblasts from SSc patients (Toyama et al., 2018). Therefore, it is not known in the first experiments whether the observed YAP1-positive cells were expressing activated YAP1. Collectively, these results show the highly specific anti-fibrotic effects of BLR-200.

Overall, results show some consistency with previous studies aimed at targeting CCN2 using the monoclonal antibody FG-3019 in an Angiotensin II (Ang II)-induced model of dermal fibrosis. (Makino et al., 2017). In this study, FG-3019 treatment mitigated Ang II-induced fibrosis, as visualized by a reduction in skin thickness, collagen deposition, and the number of αSMA-expressing myofibroblasts. FG-3019 is currently in phase III clinical trials for IPF (Clinicaltrials.gov, NCT04419558), Duchene muscular dystrophy (Clinicaltrials.gov, NCT04632940), and pancreatic cancer (Clinicaltrials.gov, NCT03941093). Since BLR-200 and FG-3019 have similar anti-fibrotic effects in dermal fibrosis, this may reflect the ability to extend the therapeutic applications of BLR-200. Considering that antibody treatments often lack efficacy in fibrotic conditions due to difficulties with tissue penetration (Brenner et al., 2016; Epenetos et al., 1986; Piersma et al., 2020), BLR-200 may represent a more viable therapeutic approach.

The bleomycin-induced model is well-established in the study of SSc dermal fibrosis (Yamamoto, 2006). After 28 days, the fibrotic lesion of injected mice adequately recapitulates the fibrotic skin of SSc patients. For that reason, this model is ideal for investigating the overall fibrotic changes prevented by BLR-200 at this endpoint. However, at this point in the model, active inflammation is not usually occurring (Yamamoto & Katayama, 2011). Therefore, a limitation of this study is that the early pro-inflammatory stages of fibrogenesis cannot be assessed. To assess the effects of BLR-200 on active inflammation, future studies should focus on earlier timepoints in the model, for example after 10 days. A more comprehensive examination of BLR-200's effects at multiple timepoints would also provide valuable insights on the mechanism of action.

The mechanism of CCN protein activity, including their exact contribution to pathological conditions such as fibrosis, has remained elusive. Considering their highly context-dependent effects, evaluation of their biological activity unquestionably requires the use of animal models. Cell culture models and in vitro assays are severely limited by their inability to recapitulate the specific components of a pathological microenvironment. Thus, to truly understand the biological significance of specific CCN proteins, for example the anti-fibrotic role of CCN3, animal models should be used. To date, very few studies have investigated the anti-fibrotic activity of CCN3 in vivo. For the first time, shown here is that a CCN3-based therapy impairs the establishment of fibrosis in a murine model of SSc dermal fibrosis, including prevention of CCN1 and CCN2 expression. This emphasizes the therapeutical potential of using CCN3-based peptides to treat fibrosis and highlights the importance of studying CCN proteins in animal models of disease.

### Example 3: Single-Cell RNA Sequencing Reveals that Therapeutic Peptides Based on CCN3 Impair Early Inflammatory and Fibrotic Changes in a Mouse Model of Systemic Sclerosis

In the experiments reported in this example, unbiased scRNA-seq and proteomic analysis investigate how fibroblasts respond to the inflammatory microenvironment in bleomycin-induced dermal fibrosis at day 10, and determine if the CCN3-derived peptide, BLR-200, can modify this response. To study this fibroblast response, transgenic mice are used to isolate and sequence COL1A2-expressing dermal fibroblasts (Bou-Gharios et al., 1996; Denton et al., 2001). This collagen-expressing fibroblast population has been shown to contribute to bleomycin-induced dermal fibrosis in a CCN2-dependent manner (Liu et al., 2014, Tsang et al., 2019). The aim is to characterize the contribution of these collagen-expressing resident dermal fibroblasts to the heterogenous fibroblast subpopulation in response to the pro-inflammatory stage of bleomycin-induced fibrosis. Understanding the heterogenous fibroblast population in fibrotic disease, and how it responds to an anti-fibrotic treatment such as BLR-200, will provide invaluable insight into the overall process of fibrogenesis.

Methods

Bleomycin-Induced Model of Dermal Fibrosis

Bleomycin sulfate (0.1 units/100 ml per injection; Sigma) or vehicle (PBS, 100 ml per injection) was injected subcutaneously into a single location on the flank of genetically modified Col1a2-Cre(ER)T; Rosa26mTmG C57BL/6J mice once daily for 10 days. Bleomycin-treated mice were further divided into two treatment groups, which were injected intraperitoneally 3 times per week with either scrambled peptide (10 μg/kg) or BLR-200 (10 μg/kg). At the end of the treatment period, the mice were sacrificed via CO₂ inhalation. Skin samples were collected for proteomic analysis, and fibroblasts were isolated from the skin of mice for single-cell RNA sequencing analysis. All animal protocols were approved by the Animal Care and Veterinary Services at Western University.

Generation of Genetically Modified Col1a2-Cre(ER)T; Rosa26mTmG Mice for Fibroblast Isolation and Proteomic Analysis

Mice were hemizygous for tamoxifen-dependent Cre recombinase under the control of the fibroblast specific proa2 collagen promoter (Col1a2-Cre(ER)-T) (Denton et al., 2001) and homozygous for a double fluorescent reporter transgene (mTmG) integrated into the Gt(ROSA)26Sor locus (Jackson Laboratories) that results in the expression of membrane-targeted tdTomato (RFP) prior to Cre-mediated excision and membrane-targeted GFP after excision. This genetically modified model has been extensively described in previous publications (Liu & Leask, 2013; Tsang et al., 2019). The Col1a2-Cre(ER)-T mice possess a fibroblast-specific enhancer, initially identified by the laboratory of Benoit de Crombrugghe (Bou-Gharios et al., 1996), subcloned upstream of the Col1a2 minimal promoter. This construct permits transgene expression specifically in fibroblasts and not in other type I collagen-expressing cells (Ponticos et al., 2004).

Polymerase chain reaction and agarose gel electrophoresis was used to genotype the DNA of the experimental animals for appropriate expression of Cre and mTmG, as previously described (Liu et al., 2013; Tsang et al., 2019). To induce membrane-targeted GFP expression in Col1a2-expressing cells, 3-week-old mice were injected intraperitoneally with a tamoxifen suspension (0.1 mL of 10 mg/mL 4-hydroxitamoxifen, Sigma) over 5 days. The mice were then subjected to bleomycin-induced fibrosis as described above in Section 4.2.1.

Isolation of Fibroblasts

To isolate fibroblasts from the skin of treated mice, skin samples from the injection site were incubated in a solution of 2 mg/mL collagenase (Gibco) in DMEM (Thermo Fisher) for 3 hours at 37° C. The connective tissue layer was then scraped off, added to 5 mL DMEM and mixed until homogenous. The solution was then centrifuged for 1 minute at 500 rpm. The supernatant layer containing cells of the connective tissue layer was then collected and centrifuged for 5 minutes at 2000 rpm. The supernatant was removed, and the cell pellet was then resuspended in a solution of 3% FBS (Gibco) in PBS and filtered through a 40 μm cell strainer (Sigma). The filtered solution was centrifuged for 5 minutes at 2000 rpm, the supernatant was discarded, and the cell pellet was resuspended in ammonium-chloride-potassium (ACK) buffer (Thermo Fisher) and incubated on ice for 10 minutes, to remove red blood cells. The cells were then centrifuged for 5 minutes at 2000 rpm and washed twice with 3% FBS in PBS, before being stained with SYTOX Blue (Thermo Fisher) as per the manufacturer's instructions. 30×10⁶ cells/mL were then subjected to fluorescence activated cell sorting (FACS) using a FACSAria III cell sorter (BD Biosciences) at the London Regional Flow Cytometry Facility. Forward- and side-scatter were used to select individual cells. A 405 nm laser was then used to select cells negative for SYTOX Blue. The remaining cells were sorted into Col1a2-lineage-positive (488 nm) and Col1a2-lineage-negative (561 nm) based on expression of GFP and tdTomato, respectively. Col1a2-lineage-positive cells (GFP+) were frozen down at −80° C. in 100% DMSO (Sigma), then sent away for single-cell RNA sequencing analysis.

Single-Cell RNA-Sequencing Analysis

Col1a2 lineage-positive (GFP+) fibroblasts from our experiment were sent for single-cell RNA sequencing by the Princess Margaret Genomics Center at the University of Toronto. Samples were prepared as outlined in 10× protocol (10× Genomics). Cells were thawed, washed, counted, and suspended in PBS (Life Technologies)+0.04% BSA (Miltenyi). Sample viability and cell counting was performed using a haemocytometer (Thermo Fisher). Following counting, the appropriate volume for each sample was calculated for a target capture of 2000 cells and loaded onto a 10× single cell B chip. After droplet generation, samples were transferred onto a pre-chilled 96 well plate (Eppendorf), heat sealed and incubated overnight in a Veriti 96-well thermocycler (Thermo Fisher). The next day, sample cDNA was recovered using Recovery Agent provided by 10× and subsequently cleaned up using a Silane DynaBead (Thermo Fisher) mix as outlined by the manufacturer's instructions. Purified cDNA was amplified for 12 cycles before being cleaned up using SPRIselect beads (Beckman Coulter). Samples were diluted 9:1 (elution buffer (Qiagen):cDNA) and run on a Bioanalyzer (Agilent Technologies) to determine cDNA concentration. cDNA libraries were prepared as outlined by the Single Cell 3′ Reagent Kits v3 user guide with modifications to the PCR cycles based on the calculated cDNA concentration.

The molarity of each library was calculated based on library size as measured by the bioanalyzer and qPCR amplification data (Sigma). Samples were pooled and normalized to 10 nM, then diluted to 2 nM using elution buffer with 0.1% Tween20 (Sigma). Each 2 nM pool was denatured using 0.1 M NaOH at equal volumes for 5 minutes at room temperature. Library pools were further diluted to 20 pM using HT-1 (Illumina) before being diluted to a final loading concentration of 14 pM. 150 ul from the 14 pM pool was loaded into each well of an 8-well strip tube and loaded onto a cBot (Illumina) for cluster generation. Samples were sequenced on a HiSeq 2500 with the following run parameters: Read 1-26 cycles, read 2-98 cycles, index 1-8 cycles.

Approximately 2000 cells were captured from each sample and subjected to 30-50,000 reads per cell. Data filtering and analysis was performed using Cell Ranger (10× Genomic, v2.1.0) and Loupe Browser Software (10× Genomics, v5.1). Genes expressed in fewer than 5 cells were removed from analysis and cells were removed if they expressed fewer than 300 genes. Data were normalized to 10,000 UMI/cell and converted to log scale. Using Loupe Browser software, a set of the most variable genes were identified, and principal component analysis (PCA) was then performed to generate Uniform Manifold Approximation and Projection for dimension reduction (UMAP) plots to visualize cell populations based on similar gene expression. Gene Ontology (GO) analysis was performed on the top 100 most representative genes per cluster, which were queried into the Gene Functional Annotation Tool from DAVID Bioinformatics Database (v6.8). The top GO terms with a p-value <0.05 were chosen to analyze fibroblast subpopulations, consistent with previously published data (Ascensión et al., 2021; Deng et al., 2021; Solé-Boldo et al., 2020; Vorstandlechner et al., 2020). Loupe Browser software was also used to generate violin plots showing the Log 2 Max Count for genes in each cluster. Specific genes known to be involved in dermal fibrosis and the inflammatory response were examined.

Proteomic Analysis

Proteomic analysis was carried out in collaboration with colleagues from the Proteomic Resource Facility (PRF) in the Department of Pathology at the University of Michigan, where mass spectrometry-based Tandem Mass Tag (TMT, Thermo Fisher) was employed. Full skin protein samples were homogenized and digested with trypsin. Protein fragments were individually labeled with one of ten isobaric mass tags following the manufacturer's protocol. After labelling, equal amounts of peptide from each condition were mixed. The labelled proteins were then fractionated by 2D-liquid chromatography, using basic pH reverse-phase separation followed by acidic pH reverse-phase. The samples were analyzed on a high-resolution, tribrid mass spectrometer (Orbitrap Fusion Tribrid, Thermo Fisher) using conditions optimized by the PRF. MultiNotch MS3 approach was employed to obtain accurate quantitation of the identified proteins. Data analysis was performed using Proteome Discoverer (v 2.3, Thermo Fisher). MS2 spectra were searched against the SwissProt reviewed mouse protein database (downloaded on 2019 Jun. 29) using the following search parameters: MS1 and MS2 tolerance were set to 10 ppm and 0.6 Da, respectively; carbamidomethylation of cysteines (57.02146 Da) and TMT labeling of lysine and N-termini of peptides (229.16293 Da) were considered static modifications; oxidation of methionine (15.9949 Da) and deamidation of asparagine and glutamine (0.98401 Da) were considered variable. Identified proteins and peptides were filtered to retain only those that passed ≤2% false-discovery rate (FDR) threshold of detection. Quantitation was performed using reporter ion intensity extracted from high-quality MS3 spectra within a ±10 PPM window centered on the theoretical m/z value of each reporter ion. Reporter ion intensities were corrected for isotopic impurities of different TMT reagents as specified by the manufacturer. Only those peptide reporter ion intensities with an average signal-to-noise ratio of 9 and <40% co-isolation interference were considered for quantification. Differential protein expression between conditions, normalizing to control (PBS) for each subject's specimens separately was established using edgeR (Robinson et al., 2010). Then, results for individual proteins from six mice per treatment were pooled. Fold change ratios were produced for either bleomycin+scrambled mice or bleomycin+BLR-200 mice, relative to PBS. Differentially expressed proteins were filtered based on a ±1.8-fold cut-off. Reactome V69 (reactome.org) was used for pathway enrichment analyses.

Results

Proteomic Analysis Reveals that BLR-200 Treatment Prevents Early Fibrotic Changes in Bleomycin-Induced Fibrosis

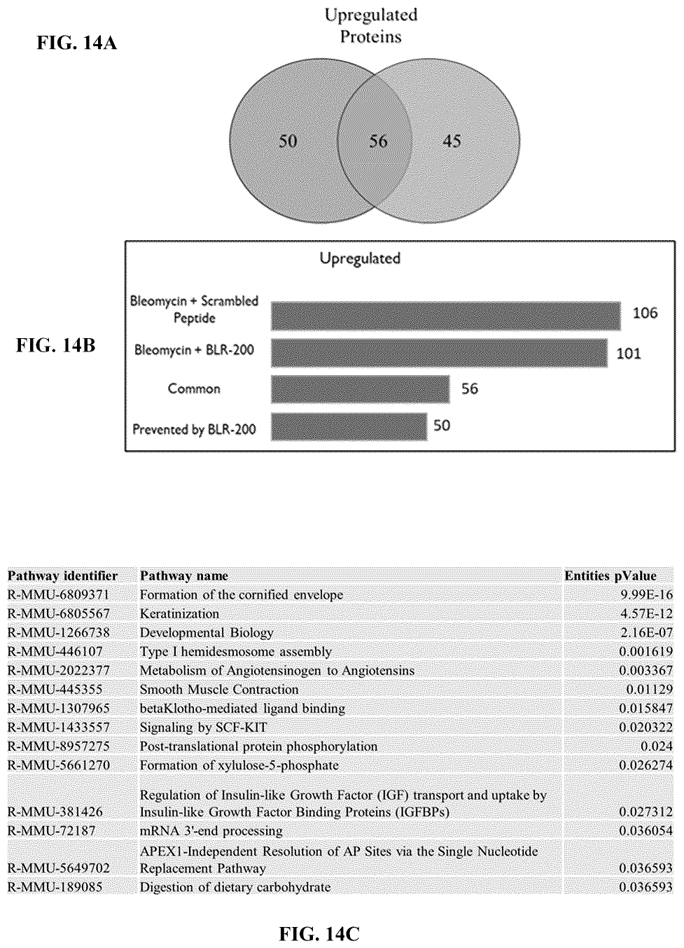

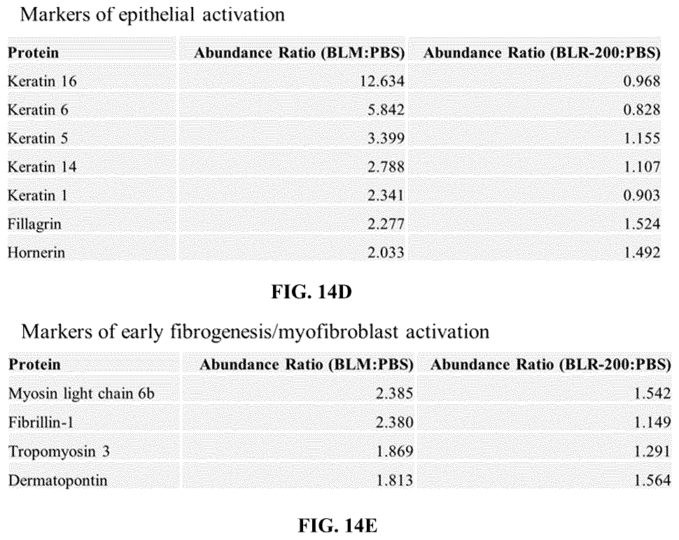

To assess the ability of BLR-200 to suppress pro-fibrotic protein expression in the initial, inflammatory stages of bleomycin-induced fibrosis, an unbiased proteomic approach was employed. C57BL/6 mice were subjected to bleomycin-induced skin fibrosis, either in the presence of a scrambled control peptide (BLM), or BLR-200 (BLR-200). Ten days after the initiation of bleomycin injection, protein was extracted from full skin and subjected to tandem mass-tag spectrometry. Protein expression for BLM mice or BLR-200 mice was normalized to protein expression in control PBS mice, producing fold change differences. A list of upregulated proteins (>1.8-fold cut-off) in each experimental group was generated. Out of the upregulated proteins in BLM mice, 50/106 were prevented by BLR-200 treatment (FIGS. 14A-14B). Proteins induced in BLM mice, but not BLR-200 mice were analyzed using the Reactome pathway database. It was found that several pathways involved in keratinocyte activation and myofibroblast contraction were prevented by BLR-200, including formation of the cornified envelope (R-MMU-6809371), keratinization (R-MMU-6805567), and smooth muscle contraction (R-MMU-445355) (FIG. 14C). Further analysis of specific proteins revealed that BLR-200 prevented several keratins known to be involved in epithelial activation (FIG. 14D). Keratinocytes are upregulated in the early stages of hyperproliferative disorders including in SSc (Aden et al., 2008; Nikitorowicz-Buniak et al., 2014) and their activation leads to production of pro-fibrotic cytokines, thereby influencing the activation of local fibroblasts (Coulombe, 1997; Lane & McLean, 2004). Keratin 1, 14, 16, and 6 are also upregulated in the epidermis of fibrotic lesions in SSc patients and keratinocyte-fibroblast interactions are abnormal in SSc (McCoy et al., 2017; Russo et al., 2021). Several protein markers of early fibrogenesis were also prevented by BLR-200 treatment (FIG. 14E). Myosin light chain 6b and tropomyosin 3 contribute to transformation of fibroblasts into myofibroblasts (Fujimura et al., 2011; Malmstrom et al., 2004). Fibrillin-1 and dermatopontin are involved in cell-matrix interactions and play important roles in ECM formation and organization (Kissin et al., 2002; Okamoto & Fujiwara, 2009). Collectively, these data suggest that BLR-200 can suppress early, inflammatory-driven changes in bleomycin-induced dermal fibrosis.

FIGS. 14A-14E. BLR-200 prevents early fibrotic changes in the proteome of bleomycin-induced dermal fibrosis.

Mice were treated with either PBS (n=3), bleomycin+scrambled peptide (BLM; n=3) or bleomycin+BLR-200 (BLR-200; n=3). Total protein was extracted from the skin and subjected to tandem mass-tag mass spectrometry. Relative protein expression for BLM mice and BLR-200 mice was normalized to the control PBS group, generating fold change differences for all detected proteins. A list of upregulated proteins in each experimental group was generated using a 1.8-fold cut-off. (FIGS. 14A-14B) Venn diagram showing that 50 out of 106 proteins induced in BLM mice are prevented by BLR-200 treatment. (FIG. 14C) Reactome pathways analysis of bleomycin-induced proteins that were prevented by BLR-200 treatment shows that the top pathways prevented involve keratinocyte activation and smooth muscle contraction. (FIG. 14D) Analysis of fold change differences for proteins that were prevented by BLR-200 treatment. Markers of epithelial activation (keratin 16, keratin 6, keratin 5, keratin 14, keratin 1, fillagrin, and hornerin), markers of early fibrogenesis (fibrillin-1 and dermatopontin) and myofibroblast activation (myosin light chain 6b and tropomyosin 3) were prevented by BLR-200 treatment.

Single-Cell RNA-Sequencing Reveals Fibroblast Heterogeneity in the Fibrotic Lesion of Experimental Mice

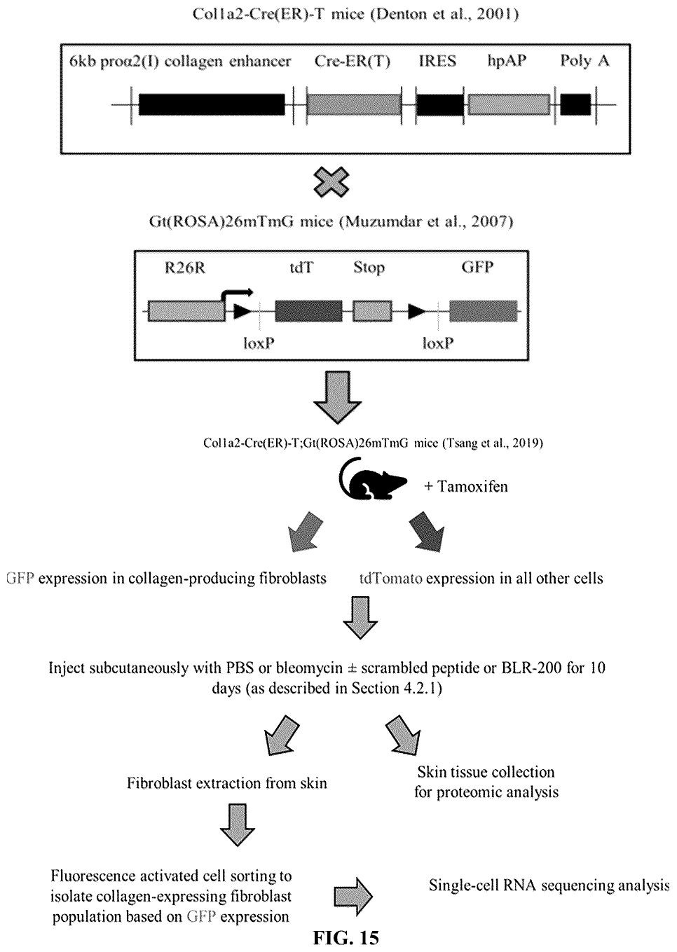

To further analyze if BLR-200 suppressed the early, inflammatory stage of bleomycin-induced fibrosis, if BLR-200 affected the ability of fibroblasts to be activated in response to the inflammatory microenvironment was assessed. Previously, it was shown that collagen-lineage fibroblasts were required for fibrogenesis (Liu, et al., 2014; Tsang et al., 2019; Tsang & Leask, 2014). To begin to address if BLR-200 could affect activation, in response to inflammation, of collagen-lineage fibroblasts, scRNA-seq was employed to first identify the collagen-linage fibroblasts subpopulations that are activated in response to inflammation. Collagen-expressing synthetic fibroblasts with GFP were labelled using a fibroblast specific Col1a2-derived promoter/enhancer (Bou-Gharios et al., 1996). Three-week-old Col1a2-Cre(ER)-T; Gt(ROSA)26mTmG mice were injected with tamoxifen to activate Cre, enabling expression of the GFP reporter in collagen-lineage fibroblasts, and mice were subsequently subjected to bleomycin-induced skin fibrosis in the presence of BLR-200 (BLR-200) or scrambled peptide (BLM) (FIG. 15). After 10 days of treatment, mice were sacrificed, and fibroblasts were extracted from the dorsal skin. Fluorescence activated cell sorting was then used to isolate GFP-expressing collagen-lineage fibroblasts. Isolated collagen-lineage fibroblasts were then subjected to scRNA-seq analysis. Quality control steps were performed by Cell Ranger to remove low quality cells (as described in Methods 4.2.5), and the transcriptomes of approximately 2000 cells from each treatment group were obtained.

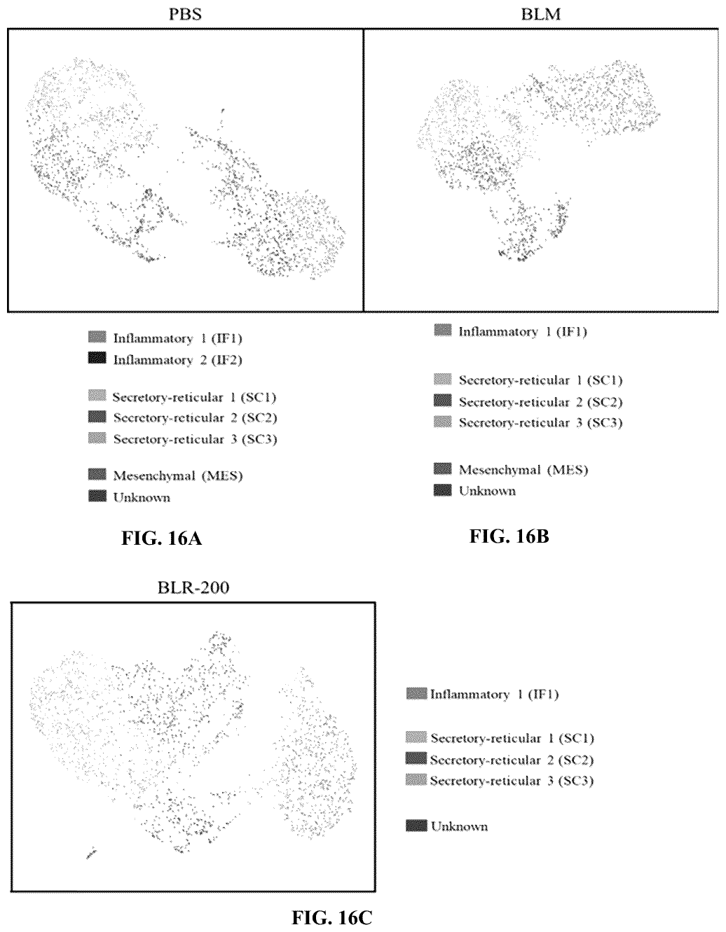

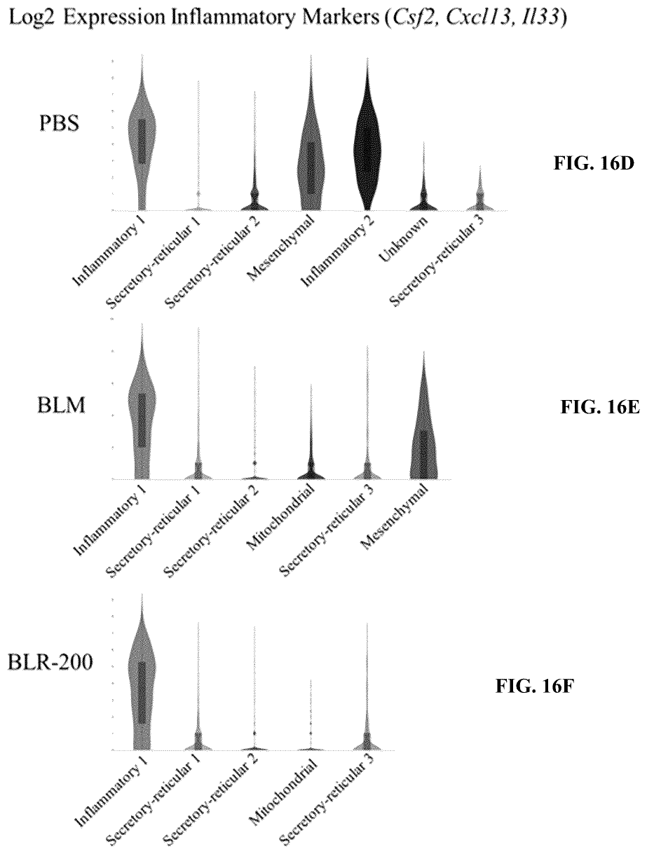

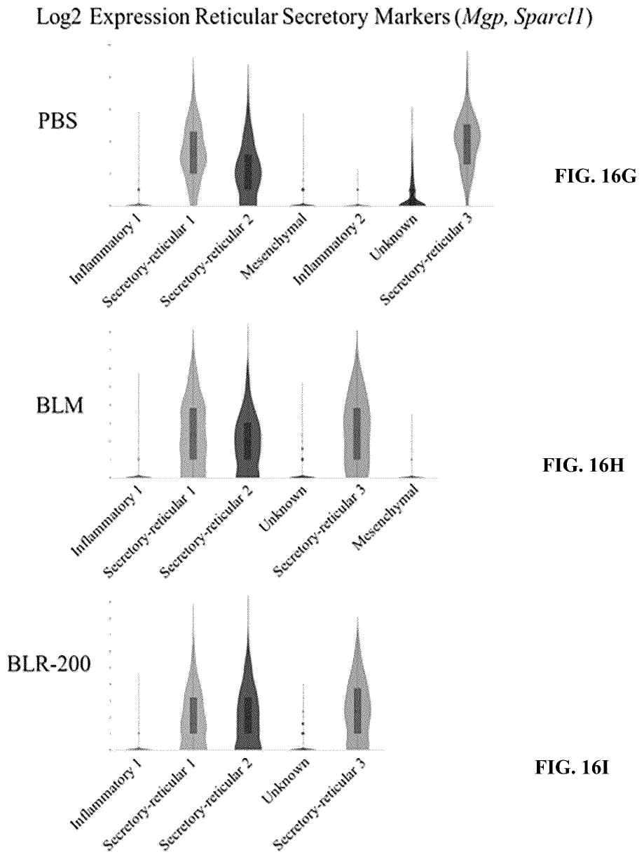

In the initial analysis, unsupervised Uniform Manifold Approximation and Projection (UMAP)-clustering revealed that collagen-lineage fibroblasts gave rise to 7 cell clusters with distinct expression profiles (FIGS. 16A-16C). It has previously been reported that, in human dermal tissue, fibroblasts can be divided into four main subpopulations based on known marker genes and functional annotations: secretory-papillary, secretory-reticular, mesenchymal, and inflammatory (Deng et al., 2021; Solé-Boldo et al., 2020). Based on these previous reports, known markers with the most representative genes in each cluster were compared and used Gene Ontology (GO) functional pathway analysis (FIG. 16A-16I) to identify the distinct fibroblast subpopulations. In the treatment groups, two inflammatory fibroblast subpopulations were identified (IF1, IF2), three secretory-reticular fibroblast subpopulations (SC1, SC2, SC3), and one mesenchymal fibroblast subpopulation (MES) (FIG. 16A-16C). One cell cluster in each treatment group had high expression of markers for mitochondrial interference and were classified as “unknown”. The inflammatory fibroblast subpopulations expressed high levels of inflammatory markers C-X-C motif chemokine ligand 13 (Cxcl13), colony stimulating factor 2 (Csf2), and interleukin-33 (Il33) (FIGS. 16D-16F). GO functional pathway analysis for these populations showed enriched cytokine- and chemokine-mediated signaling, neutrophil chemotaxis, positive regulation of chemokine production, and positive regulation of prostaglandin secretion (Supplementary FIG. 4-1A-C). The reticular secretory fibroblast subpopulations expressed high levels of secretory markers matrix Gla protein (Mgp) and sparc-like 1 (Sparcl1) (FIGS. 16G-16F). GO functional pathway analysis for these populations showed enriched extracellular matrix organization, protein folding, positive regulation of cell adhesion, and collagen fibril formation (FIGS. 16A-16f). Collectively, transcriptomic analysis of fibroblast subpopulations in the fibrotic lesions of experimental mice reveals specific fibroblast heterogeneity have been shown consistent with previously reported literature.

FIG. 15. Isolation of collagen-lineage fibroblasts from the fibrotic lesion of mice subjected to bleomycin-induced dermal fibrosis.

Illustration of workflow for labeling and isolation of collagen-lineage fibroblasts for scRNA-seq analysis. Collagen-lineage fibroblasts were labeled with GFP using a fibroblast specific collagen promoter/enhancer. Briefly, experimental mice were hemizygous for tamoxifen-dependent Cre recombinase under the control of the fibroblast specific proa2 collagen promoter (Col1a2-Cre(ER)-T; Denton et al., 2001) and homozygous for a double fluorescent reporter transgene (mT/mG) integrated into the Gt(ROSA)26Sor locus (GT(ROSA)26mTmG; Muzumdar et al., 2007). At 3 weeks of age, mice were injected with tamoxifen to induce GFP expression. After two weeks, bleomycin sulfate (0.1 units/100 μl per injection) or PBS (100 μl per injection) was injected subcutaneously into a single location on the flank of the mice once daily for 10 days. Bleomycin-treated mice were further divided into two treatment groups, which were injected intraperitoneally 3 times per week with either 10 μg/kg scrambled peptide or 10 μg/kg BLR-200. At the conclusion of the experiment, dermal tissue from the fibrotic lesion was collected. Fibroblasts were extracted from the dermis and GFP-expressing cells were isolated by fluorescence activated cell sorting before being subjected to scRNA-seq analysis.

FIGS. 16A-16. Identification of collagen-lineage fibroblast subpopulations in bleomycin-induced dermal fibrosis.

Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. (FIGS. 16A-16C) Unsupervised UMAP clustering of cells in each treatment group yielding a total of 7 distinct clusters among the different treatments, comprising 2 inflammatory fibroblast clusters (IF1, IF2), 3 reticular-secretory fibroblast clusters (SC1, SC2, SC3), 1 mesenchymal fibroblast cluster (MES), and 1 unknown cluster. (FIGS. 16D-16F) Violin plots of inflammatory fibroblast marker genes for cell type identification. Inflammatory fibroblast markers include: Cxcl13, Csf2, Il33. (FIGS. 16G-16F) Violin plots of reticular-secretory fibroblast marker genes for cell type identification. Reticular-secretory fibroblast markers include: Mgp and Sparcl1.

BLR-200 Treatment Prevents the Bleomycin-Induced Increase in Il6 and Cxcl2 Expression in the Inflammatory Fibroblast Subpopulation

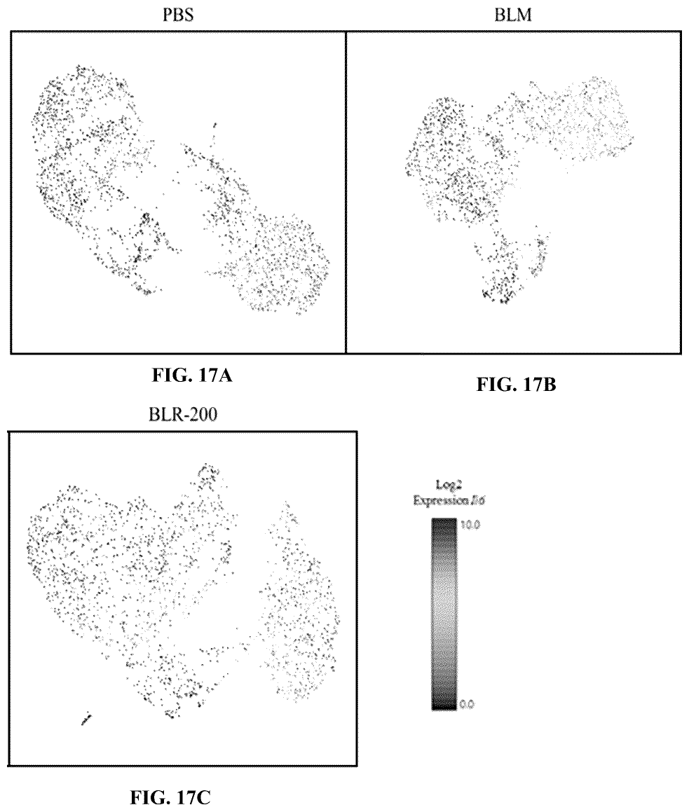

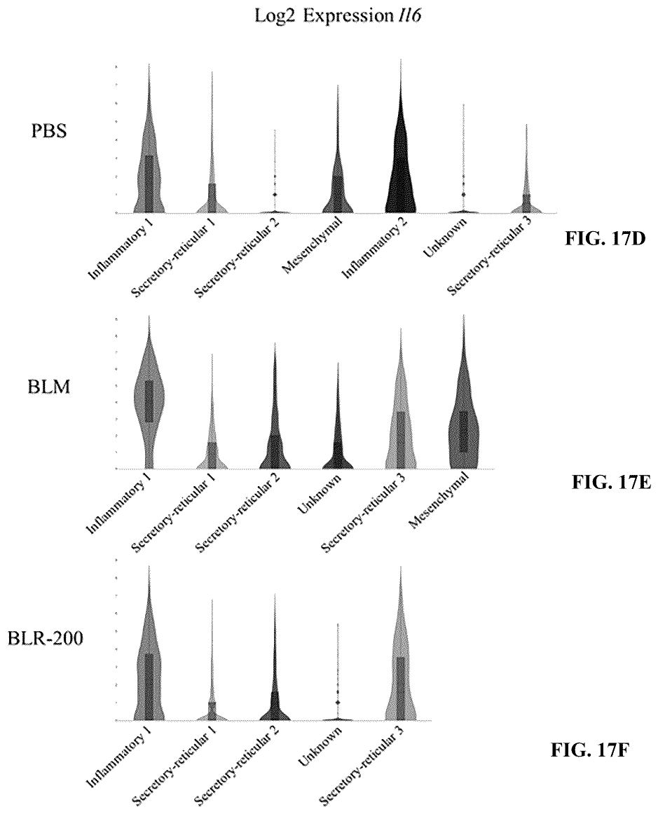

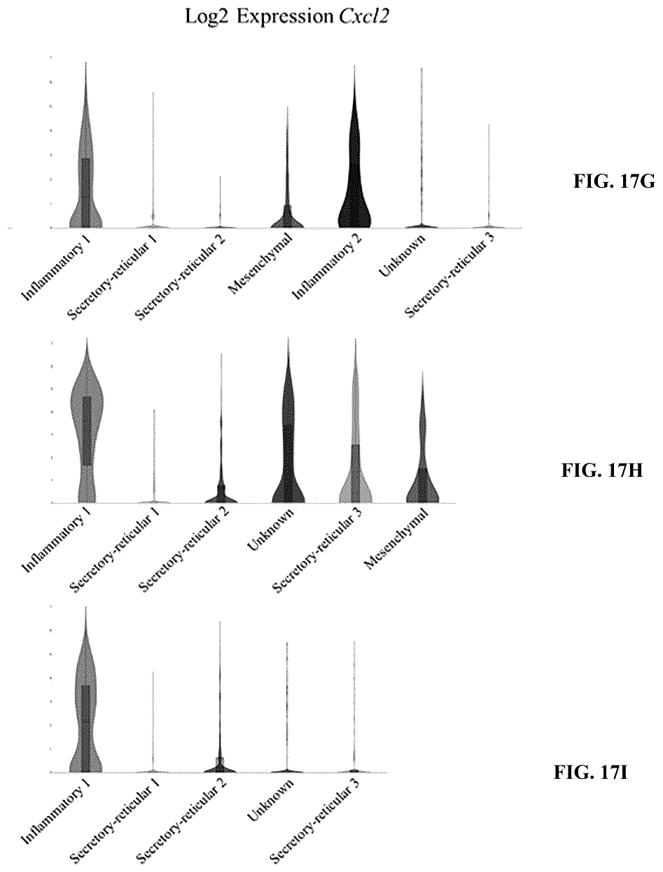

The transcriptomic changes were assessed occurring in the fibroblast subpopulations among treatment groups. It was clear from the UMAPs that there was a shift in the inflammatory fibroblast subpopulation (IF1) relative to the reticular secretory subpopulations (FIG. 16A-16C). Thus, the gene expression changes were determined that were causing this shift. The list of most variable genes and identified that interleukin-6 (Il6) and Cxcl2 were expressed at higher levels in the IF1 inflammatory subpopulation of BLM mice compared to PBS; however, this increase was impaired in the IF1 inflammatory subpopulation of BLR-200 mice (FIG. 17A-17C; FIGS. 17D-17I). IL-6 plays a pivotal role in driving acute inflammation and has also been shown to contribute to unresolved inflammation in fibrosis, including in SSc (Fielding et al., 2014; Johnson et al., 2020; Kawaguchi, 2017; Khan et al., 2012). CXCL2 is a chemokine that plays a role in many inflammatory processes and is also upregulated in the dermal fibroblasts of SSc patients (Johnson et al., 2015; Sahin & Wasmuth, 2013). Altogether, these data indicate that BLR-200 prevented pro-inflammatory transcriptomic changes in collagen-lineage dermal fibroblasts responding to bleomycin-induced dermal fibrosis.

FIG. 17A-17I. BLR-200 prevents the bleomycin-induced transcriptomic changes in the inflammatory fibroblast subpopulation.

Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. (FIGS. 17A-17C) Unsupervised UMAP clustering of cells reveals that BLM mice have a shift in the inflammatory subpopulation. The inflammatory subpopulation of BLM mice expresses higher levels of the pro-fibrotic, pro-inflammatory IL6. Cells are coloured based on Log 2(Il6 expression). (FIGS. 17D-17I) Violin plots showing Log 2 expression of Il6 and Cxcl2. BLR-200 prevents the bleomycin-induced increase in gene expression of Il6 and Cxcl2 in the inflammatory fibroblast subpopulation.

BLR-200 Prevents Expression of an NLRP3 Inflammasome-Related Gene Signature

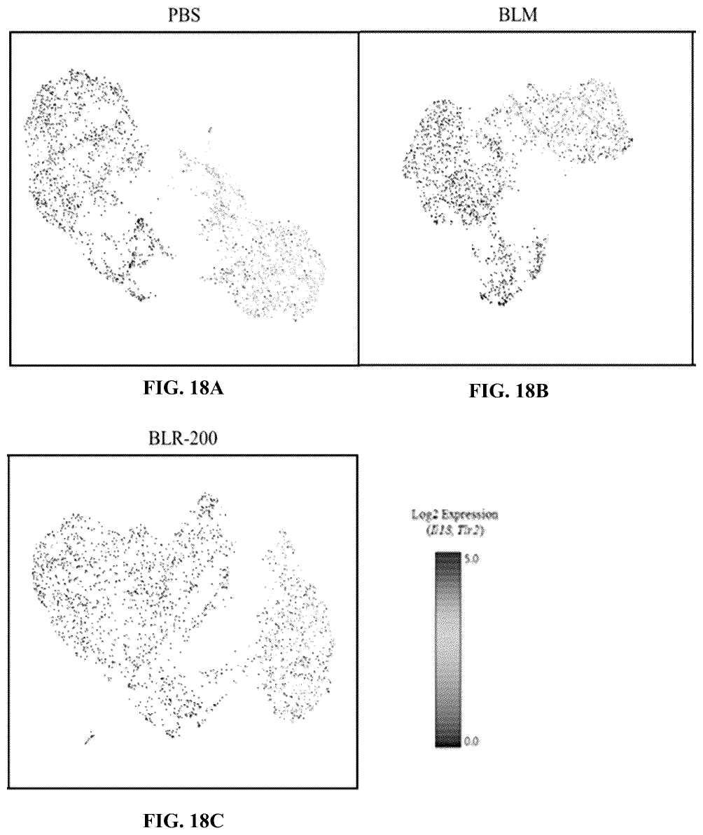

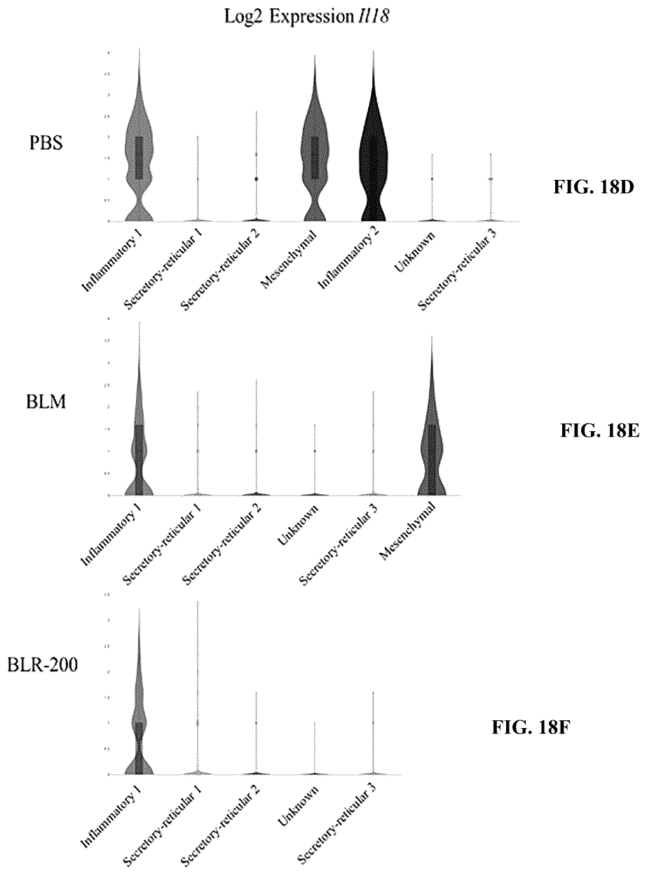

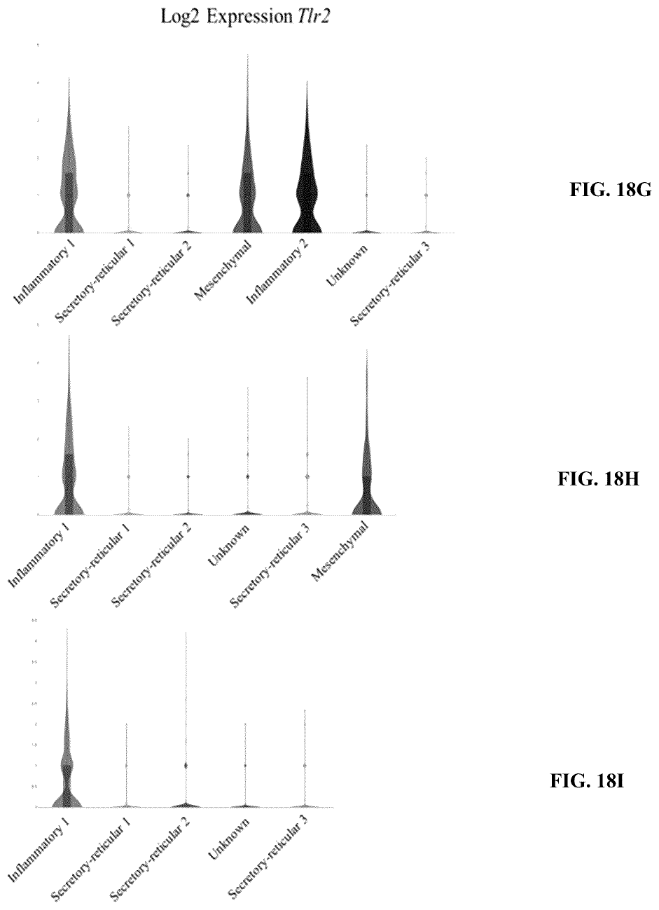

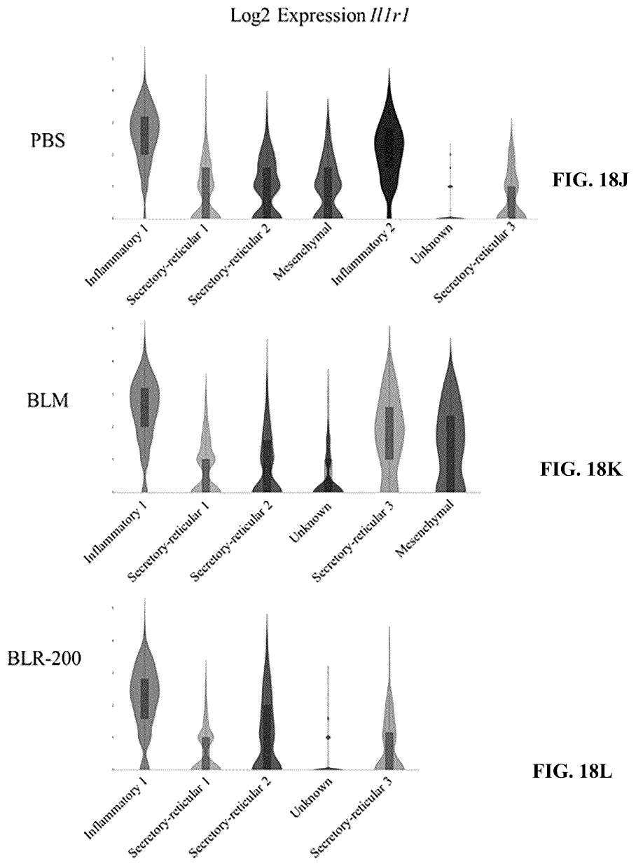

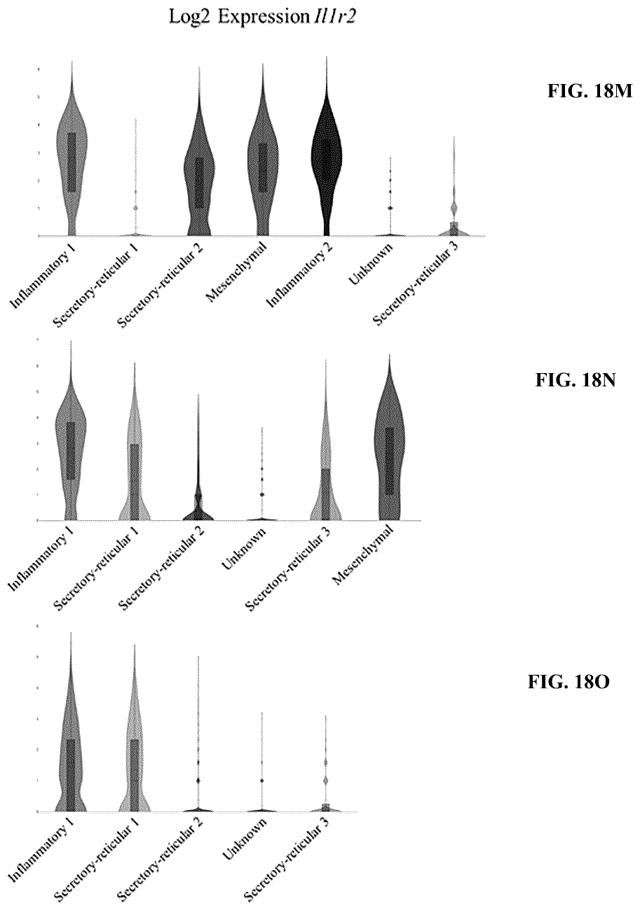

To continue the assessment of BLR-200 on bleomycin-induced pro-inflammatory changes among fibroblast subpopulations, the list of most variable genes for each cluster and used violin plots to compare expression of several genes of interest. As exhibited by UMAPs (FIG. 18A-18C) and violin plots, BLR-200-treated mice had impaired expression of interleukin-18 (Il18), toll-like receptor 2 (Tlr2), interleukin-1 receptor type 1 (Il1r1), and interleukin-1 receptor type 2 (Il1r2) (FIGS. 18D-18F; 18G-18I; 18J-18L; 18M-18O). This impairment was most obvious in the IF1 inflammatory subpopulation. This genetic signature is associated with activation of the NLRP3 inflammasome, which has been implicated in fibrogenesis in the liver, kidney, and lung (Colak et al., 2021; Hsu et al., 2021; Rastrick & Birrell, 2014). Moreover, activation of the inflammasome also mediates the early innate immune response in patients with SSc, and an inflammasome-related gene signature is associated with a more aggressive fibroblast in SSc (Artlett et al., 2011; Henderson et al., 2018; Martinez-Godinez et al., 2015). These results provide further evidence that BLR-200 can impair the early, inflammatory changes of bleomycin-induced dermal fibrosis.

FIGS. 18A-18O. BLR-200 Prevents Expression of an NLRP3 Inflammasome-Related Gene Signature.

Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. (FIGS. 18A-18C) Unsupervised UMAP clustering of cells reveals that expression of NLRP3 inflammasome markers Il18 and Tlr2 is lower in BLR-200 treated mice. Cells are coloured based on Log 2(Il18 and Tlr2 expression). Violin plots also reveal that BLR-200 impairs expression of inflammasome-related markers (FIGS. 18D-18F) Il18, (FIGS. 18G-18I) Tlr2, (FIGS. 18J-18L) Il1r1, and (FIGS. 18M-18O) Il1r2.

BLR-200 Specifically Prevents the Bleomycin-Induced Increase in the Pro-Fibrotic Transcription Factor Egr1

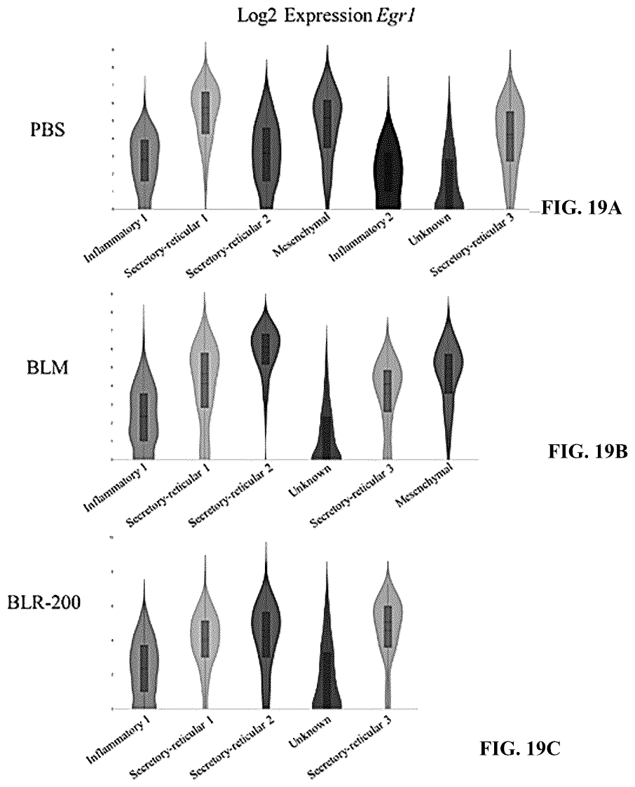

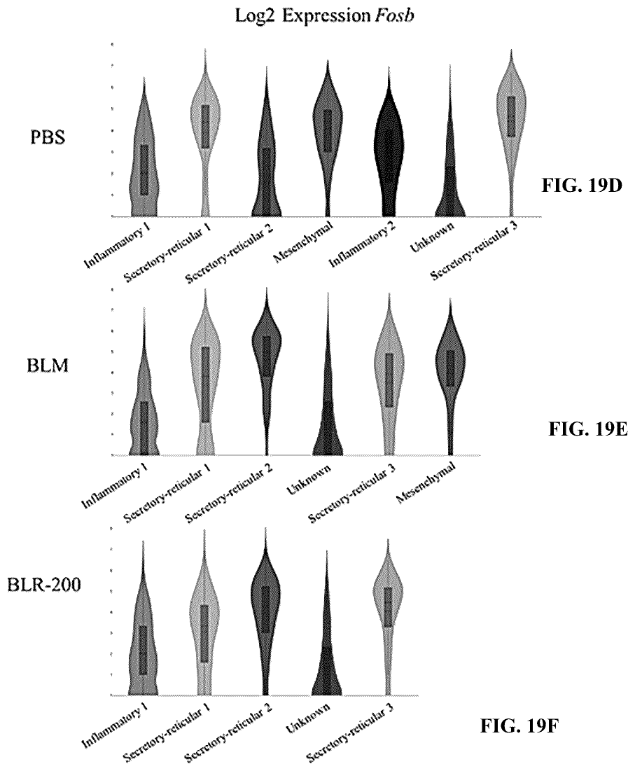

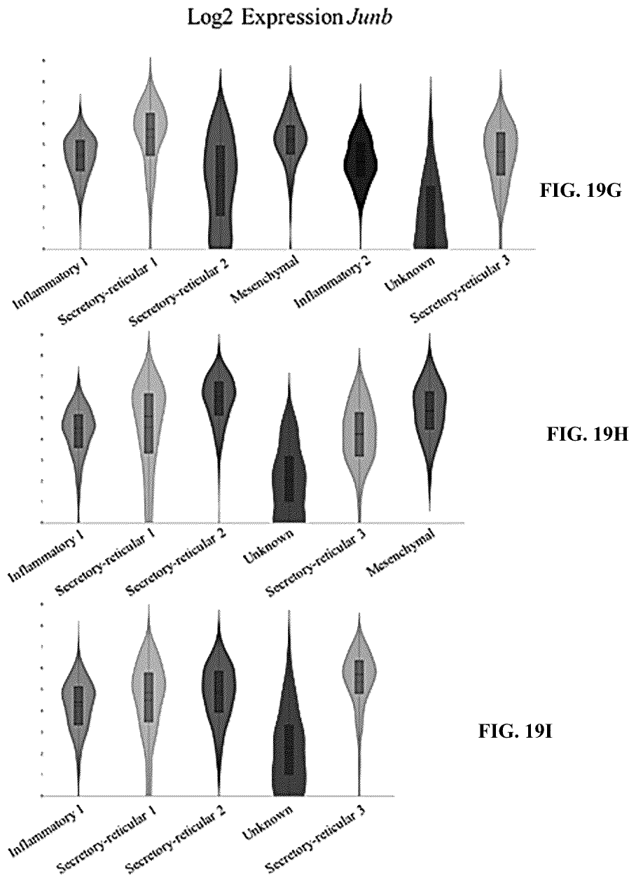

To further assess the ability of BLR-200 to prevent early, inflammatory changes in bleomycin-induced dermal fibrosis, transcriptomic changes in expression of pro-fibrotic transcription factors were investigated. Once again, the list of most variable genes were analyzed, and used violin plots to compare the expression of several genes of interest. In the secretory-reticular (SC2) fibroblast subpopulation of BLM mice, expression of early growth response-1 (Egr1) was increased; a change that was prevented by BLR-200 treatment (FIG. 19A-19C). EGR1, a multifunctional transcription factor, promotes fibroblast activation and fibrotic gene expression; an EGR1 gene expression signature is upregulated in a cohort of dcSSc patients (Bhattacharyya et al., 2013). EGR1 also mediates the early influx of inflammatory cells into fibrotic lesions and plays a role in regulating TGFβ activity in pathological matrix remodeling (Wu et al., 2009).

To assess whether BLR-200 specifically suppressed Egr1 or more generally impaired expression of transcription factors known to promote fibrosis, if addition of BLR-200 also impeded as investigated the increased expression of members of the Activator Protein 1 (AP1) transcription factor complex (Avouac et al., 2012; Ponticos et al., 2009; Wernig et al., 2017). However, BLR-200 had minimal effects on bleomycin-induced increase in Fosb and Junb (FIG. 19D-19F). These results are consistent with the notion that BLR-200 selectively affects particular aspects of the fibroblast activation in response to inflammatory stimuli, and thus would appear to represent an extremely specific anti-fibrotic drug.

FIG. 19A-19I. BLR-200 has specific effects on pro-fibrotic transcription factors.

Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. (FIGS. 19A-19C) Violin plot showing Log 2 expression of Egr1. BLR-200 prevents the bleomycin-induced increase in gene expression of Egr-1 in the Secretory-reticular (SC2) subpopulation. Violin plots of Activator protein (AP1) transcription factor complex components, showing Log 2 expression of (FIGS. 19D-19F) Fosb and (FIGS. 19G-19I) Junb.

BLR-200 Prevents the Appearance of a Mesenchymal Fibroblast Subpopulation

After identifying multiple fibroblast subpopulations in each treatment group, a mesenchymal fibroblast subpopulation (MES) was present in PBS and BLM mice, but not in BLR-200 treated mice. This mesenchymal subpopulation was characterized by expression of genes involved in development of the vasculature, skeletal system, and connective tissue (Ascensión et al., 2021; Deng et al., 2021; Solé-Boldo et al., 2020) (Supplementary FIG. 4-1A-B). This subpopulation was confirmed in the study using UMAPs (FIG. 20A-20C and violin plots (FIG. 20D-20L) looking at expression of angiopoietin-1 receptor (Tek), protein odd-skipped-related 2 (Osr2), and secreted frizzled-related protein 2 (Sfrp2), which are all involved in mesenchymal processes (Ascensión et al., 2021; Solé-Boldo et al., 2020). In BLR-200 mice, these markers did not appear at a significant level in any of the identified subpopulations. Additionally, this mesenchymal subpopulation expressed Wnt family member 10b (Wnt10b) and endothelin-1 (Edn1), both of which have been implicated in fibrogenesis in SSc (Chadli et al., 2019; Fonseca et al., 2006; Kogak et al., 2020) (FIG. 20M-20O; 20P-20R). This is consistent with recent evidence suggesting that a mesenchymal fibroblast subpopulation is crucial in fibrotic skin diseases such as SSc (Deng et al., 2021). These results therefore indicate that BLR-200 treatment may prevent the formation of a potentially pro-fibrotic mesenchymal-like fibroblast subpopulation.

FIG. 20A-20R. BLR-200 Prevents the Appearance of a Mesenchymal Fibroblast Subpopulations.

Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. (FIGS. 20A-20C) Unsupervised UMAP clustering of cells reveals that expression of mesenchymal markers Tek, Osr2, and Sfrp2 are not appreciably present in BLR-200 treated mice. Cells are coloured based on Log 2 expression of Tek, Osr2, and Sfrp2. (FIGS. 20D-20L) Violin plots showing Log 2 expression of Tek, Osr2 and Sfrp2. BLR-200 treated mice do not appreciably express these mesenchymal markers in any fibroblast subpopulations. Violin plots showing Log 2 expression of (FIG. 20M-20O) Wnt10b and (FIGS. 20P-20R) Edn1. BLR-200 treatment prevents expression of these pro-fibrotic genes.

FIG. 21A-210, Functional Pathway Assessment of Fibroblast Subpopulations Identified in scRNA-Seq Analysis

Mice were treated with either PBS (n=3), bleomycin+scrambled peptide (BLM; n=3) or bleomycin+BLR-200 (BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. Graphs showing the top enriched Gene Ontology (GO) terms in each fibroblast subpopulation, sorted by p-value. (FIGS. 21A-21F) PBS, (FIGS. 21G-21K) BLM, and (FIGS. 21L-21O) BLR-200.

Discussion

Currently, there is no approved therapy for treating the fibrosis observed in SSc. This deficiency can be attributed to multiple factors, including: (a) the etiology of SSc is relatively unknown; (b) knowledge gaps in the understanding of fibrogenesis; and (c) a lack of well-defined in vivo animal models to test potential anti-fibrotic therapies. The CCN family of matricellular proteins has emerged as an important player in fibrogenesis (Leask, 2020). CCN1 and CCN2, the most-studied members of the family, are upregulated in the connective tissue of early-onset SSc patients, and have pro-fibrotic effects (Sonnylal et al., 2010). CCN1 plays a role in collagen organization and has pro-inflammatory activity (Lau, 2011; Quesnel et al., 2019). CCN2 promotes a pro-fibrotic microenvironment by contributing to the persistent activation of myofibroblasts (Liu et al., 2011; Tsang et al., 2019). In contrast, CCN3 is downregulated in SSc (Xu Shi-wen and Richard Stratton, unpublished data), and has specific anti-fibrotic effects in kidney fibrosis (Riser et al., 2014). In this example, targeting the regulation and activity of CCN proteins using BLR-200, a proprietary CCN3-derived peptide, can suppress specific early inflammatory and pro-fibrotic responses in the bleomycin-induced model of SSc dermal fibrosis. Furthermore, scRNA-seq defines the heterogenous collagen-lineage fibroblast subpopulation in the early inflammatory stage of bleomycin-induced fibrosis.

To investigate the early inflammatory stage of bleomycin-induced fibrosis, tissue obtained 10 days post-initiation of bleomycin injection was analyzed. This stage of the model precedes major fibrotic changes in the microenvironment, such as excessive collagen deposition (Yamamoto & Katayama, 2011). Early pro-fibrotic changes, such as keratinocyte and myofibroblast activation usually occur at this stage of fibrogenesis (Gabbiani, 2003; McCoy et al., 2017). the proteomic analysis revealed that BLR-200 treatment prevented these early pro-fibrotic changes. Notably, markers of keratinocyte activation (keratin 1, 14, 15, 6) were prevented by BLR-200 treatment. These keratins are upregulated in the epidermis of fibrotic lesions in SSc patients (Aden et al., 2008; Nikitorowicz-Buniak et al., 2014). Previous studies have also shown that activated SSc keratinocytes likely play a role in early myofibroblast activation, through release of pro-fibrotic cytokines that can prime fibroblasts to become activated and to be hyper-reactive to fibrotic stimuli (Aden et al., 2010; McCoy et al., 2017). Thus, impairing keratinocyte activation is an important anti-fibrotic effect elicited by BLR-200. Protein expression of other markers of early fibrogenesis and myofibroblast activation were also prevented by BLR-200, including fibrillin-1, dermatopontin, myosin light chain 6b, and tropomyosin 3 (Fujimura et al., 2011; Kissin et al., 2002; Malmstrom et al., 2004; Okamoto & Fujiwara, 2009). Collectively, these data indicate that BLR-200 can inhibit the increased inflammation-driven pro-fibrotic protein expression observed 10 days post-initiation of bleomycin injection.

Given the fact that, in SSc, keratinocyte activation precedes fibrotic changes in the microenvironment and promotes myofibroblast differentiation (Aden et al., 2010; McCoy et al., 2017), it is likely that fibroblasts within the lesional dermis are just starting to become activated 10 days post-initiation of bleomycin injection. Investigation of the transcriptomic changes occurring in fibroblasts at this point, and how they are affected by BLR-200, would therefore provide valuable information on potential mechanisms underlying fibrogenesis.

Thus, unbiased scRNA-seq analysis to investigate the specific effects of BLR-200 on the heterogenous myofibroblast population and how it responds to early inflammatory changes in bleomycin-induced dermal fibrosis. Several recent studies have used scRNA-seq to characterize major fibroblast subpopulations in the dermis (Deng et al., 2021; Solé-Boldo et al., 2020). Solé-Boldo and colleagues sought to decipher fibroblast heterogeneity in aging human skin. The authors isolated and sequenced total cells from full-thickness skin samples. In young, healthy skin, they identified a fibroblast population that could be subdivided into four major subpopulations showing differential functional annotations. These subpopulations include an inflammatory subset, a secretory-reticular subset, a secretory-papillary subset, and a mesenchymal subset (Solé-Boldo et al., 2020). In a similar study, Deng and colleagues confirmed the presence of these four main subpopulations in human keloid scars (Deng et al., 2021). One caveat is that neither of these studies examined the role of these fibroblast subpopulations in active fibrosis. Furthermore, they were unable to make conclusions on the origin of these fibroblast subpopulations. Thus, their contributions to fibrogenesis remain to be determined. the study contributes to this gap in knowledge. A GFP reporter construct to postnatally label resident Col1a2-expressing dermal fibroblasts. This population has been shown to contribute to the activated myofibroblast population in bleomycin-induced fibrosis (Liu et al., 2014; Tsang et al., 2019). Labelling these “pre-myofibroblasts” before they become activated provides distinct advantages in studying cellular changes in the early stages of fibrogenesis. the scRNA-seq analysis revealed that, in a murine model of dermal fibrosis, collagen-expressing dermal fibroblasts can give rise to fibroblast subpopulations that closely resemble previously identified inflammatory, secretory-reticular, and mesenchymal subpopulations. Notably, a secretory-papillary subpopulation was not observed. This was not unexpected, as collagen-lineage myofibroblasts, which actively contribute to fibrosis, are not generally associated with the papillary dermis (Driskell & Watt, 2015).

To identify the fibroblast subpopulations in the experiment, functional pathway analysis was used, which provides a consistent guideline for functional annotation and subsequent fibroblast identification. In this report, the inflammatory subpopulations were enriched for pathways involved in cytokine- and chemokine-mediated signaling, neutrophil chemotaxis, positive regulation of chemokine production, and positive regulation of prostaglandin secretion. The secretory-reticular fibroblast subpopulations were enriched for pathways involved in extracellular matrix organization, protein folding, positive regulation of cell adhesion, and collagen fibril formation. Finally, the mesenchymal fibroblast subpopulations were enriched for pathways involved in skeletal system development, regulation of vasculature development, connective tissue development and cartilage development. Expression of known markers for inflammatory (Csf2, Cxcl13, Il-33), secretory-reticular (Mgp, Sparcl1) and mesenchymal (Tek, Osr2, Sfrp2) fibroblasts to provide further confirmation for this classification (Deng et al., 2021; Philippeos et al., 2018; Valenzi et al., 2019; Vorstandlechner et al., 2020). Although these markers differ slightly from previous scRNA-seq studies, this is likely due to species differences and general differences in experimental approach. Collectively, these data builds upon previous experiments investigating fibroblast heterogeneity (Deng et al., 2021; Solé-Boldo et al., 2020) and extends their classification of fibroblast subpopulations into a murine model of dermal fibrosis. Furthermore, investigation of BLR-200's specific effect on these fibroblast subpopulations will allow us to determine the exact contribution of these cell types to dermal fibrosis.

One of the most obvious changes observed in the scRNA-seq data was the bleomycin-induced shift in the inflammatory fibroblast subpopulation (IF1), which was at least partially prevented by BLR-200. A bleomycin-induced Il-6 Cxcl2 expression pattern was contributing to this shift in the inflammatory subpopulation. IL-6 is a known pro-fibrotic cytokine and has long been recognized as an early marker of dermal fibrosis in SSc (Feghali et al., 1992; Hasegawa et al., 1998). Previous studies have also shown that prevention of IL-6 signalling attenuates bleomycin-induced dermal fibrosis (Kitaba et al., 2012). CXCL2 has been shown to play a role in multiple forms of fibrosis and is also upregulated in SSc (Johnson et al., 2015; King et al., 2018; Sahin & Wasmuth, 2013). Thus, impairment of these pro-fibrotic cytokines represents an important early step in prevention of fibrogenesis. Collectively, these data are consistent with the hypothesis that BLR-200 can prevent early pro-inflammatory changes in bleomycin-induced fibrosis.

Previous experiments have revealed the critical involvement of the NLRP3 inflammasome in promoting the early inflammatory events that result in fibrosis (Colak et al., 2021; Hsu et al., 2021; Martinez-Godinez et al., 2015). The NLRP3 inflammasome is a group of multimeric protein complexes involved in the innate immune response (Kelley et al., 2019). Toll-like-receptors (TLRs) play a role in activating intracellular NLRP3 inflammasome proteins, which results in activation and secretion of IL-18 and IL-10 (Henderson et al., 2018). In SSc, NLRP3 inflammasome-related genes are overexpressed, including IL18, IL1β, and TLRs (Broen et al., 2012; Martinez-Godinez et al., 2015). Moreover, NLRP3 inflammasome activation has been shown to contribute to myofibroblast activation and collagen secretion in fibroblasts (Artlett et al., 2011, 2017). Artlett and colleagues also showed that the NLRP3 inflammasome plays a crucial role in development of bleomycin-induced dermal fibrosis (Artlett et al., 2011). the scRNA-seq data reveals that BLR-200 impaired expression of NLRP3 inflammasome markers including Il18, Tlr2, Il1r1, and Il1r2. Given that activation of the inflammasome is a crucial early inflammatory change in fibrosis, this further emphasizes the ability of BLR-200 to impair fibrogenesis. To date, no studies have examined the direct relationship between CCN proteins and inflammasome activity in pathological fibrosis. Therefore, it is not known whether the effects of BLR-200 on inflammasome activation are direct or indirect. However, CCN2 and inflammasome-related genes have been shown to be co-expressed in cardiac fibrosis, suggesting a potential relationship (Chandra et al., 2021).

Upon further assessment of scRNA-seq data, BLR-200 also prevents the bleomycin-induced increase in Egr1 expression. This was most apparent in the secretory-reticular fibroblast subpopulation (SC2). EGR1 is a transcription factor that responds to cellular stress, integrating extracellular signals and orchestrating early-immediate cellular responses to injury (Bhattacharyya, Wu, et al., 2011). EGR1 is overexpressed in the fibrotic tissue of SSc patients and induces an “EGR1 responsive gene signature” that is enriched in an inflammatory subset of dcSSc patients, thus likely plays a role in mediating the early inflammatory stage of fibrosis (Bhattacharyya, Sargent, et al., 2011). Moreover, Wu and colleagues showed that Egr1-null mice had an impaired inflammatory response and attenuated dermal fibrosis in response to bleomycin (Wu et al., 2009). Expression levels of other transcription factors known to be involved in fibrosis, including the AP1 transcription factor complex (Avouac et al., 2012). scRNA-seq analysis revealed increased expression of Fosb and Junb in response to bleomycin, however BLR had no observable effects on this change. Although unexpected, these results are consistent with the notion that BLR-200 selectively affects particular aspects of fibroblast activation, thus would appear to represent an extremely specific anti-fibrotic drug. The fact that EGR1 likely plays a role in mediating the early inflammatory stage of fibrogenesis is also significant in this case.

Another intriguing observation from the scRNA-seq data was the absence of the mesenchymal fibroblast subpopulation in BLR-200 treated mice. This subpopulation was characterized based on the expression of Tek, Osr2, and Sfrp2, and enrichment of functional pathways involved in development and differentiation (Deng et al., 2021; Solé-Boldo et al., 2020). The significance of this subpopulation in the context of fibrosis is not exactly clear. Although this mesenchymal subpopulation does not appear to display “the traditional” pro-fibrotic expression patterns, it does express Wnt10b and Edn1, both of which have been implicated in SSc fibrogenesis (Fonseca et al., 2006; Kogak et al., 2020). Furthermore, other scRNA-seq studies have reported that a mesenchymal fibroblast subpopulation is implicated in the fibrogenesis (Deng et al., 2021). Moreover, mesenchymal-like fibroblasts may be “primed” to induce a myofibroblast signature in response to the fibrotic microenvironment in SSc (Taki et al., 2020). Thus, the ability of BLR-200 to prevent this mesenchymal fibroblast subpopulation may have important implications on fibrogenesis in SSc.

In this example, unbiased scRNA-seq analysis to identify multiple fibroblast subpopulations in a model of dermal fibrosis that are consistent with previous observations (Ascensión et al., 2021; Solé-Boldo et al., 2020). These findings contribute to the overall understanding of fibroblast heterogeneity in bleomycin-induced dermal fibrosis. Furthermore, this study has built a base for further, more detailed analyses aimed at elucidating the exact roles of the diverse fibroblast populations in dermal fibrosis.

This example also reveals that that BLR-200, a CCN3-derived peptide, effectively prevented early pro-inflammatory changes in collagen-lineage fibroblasts and had specific inhibitory effects on the pro-fibrotic transcription factor EGR1. Preventing these changes likely impairs the formation of a pro-fibrotic microenvironment that permits activation and perpetuation of myofibroblasts. Thus, for the first time, that CCN3 has anti-fibrotic effects, through impairment of the early inflammatory response in collagen-lineage fibroblasts, in an in vivo model of dermal fibrosis. This has widespread implications for the study of CCN proteins in fibrosis and further emphasizes the importance of studying this family of matricellular proteins in animal models.

Nevertheless, BLR-200 prevents early pro-inflammatory and pro-fibrotic changes in a murine model of SSc dermal fibrosis, emphasizing the therapeutic and diagnostic potential of using CCN3-based peptides and drugs to treat fibrosis.

### Example: BLR-200 Impairs Activation of Collagen-Lineage Fibroblasts by Bleomycin

FIGS. 22A-22O show that BLR-200 impairs activation of collagen lineage fibroblasts. Mice were treated with either PBS (FIGS. 22A, 22D, 22G, 22J, n=3), bleomycin+scrambled peptide (FIGS. 22B, 22E, 22H, 22K, BLM; n=3) or bleomycin+BLR-200 (FIGS. 22C, 22F, 22I, 22L, BLR-200; n=3) for 10 days. At the conclusion of the experiment, collagen-lineage fibroblasts were extracted and isolated by FACS. Isolated cells were subjected to scRNA-seq analysis and approximately 2000 cells from each treatment were sequenced. FIGS. 22A-22C Unsupervised UMAP clustering of cells reveals that expression of collagen lineage fibroblasts COL15A1 and PI16 is impaired in BLR-200 treated mice. Cells are colored based on Log 2(COL15A1 and PI16 expression). Violin plots also reveal that BLR-200 impairs activation of collagen-lineage fibroblasts COL15A1, (FIG. 22F) PI16, (FIG. 22I) C3, (FIG. 22L). Collagen-lineage fibroblasts are universal fibroblasts. 10-day bleomycin/BLR200 collagen 1A2 (GFP)-lineage fibroblast scRNAseq. All 3 clusters are “universal fibroblast”. N=3, 2000 cells (pooled), 30-50K reads/cell.

FIGS. 23A-23C show Unsupervised UMAP clustering of cells reveals of 10-day bleomycin/BLR200 GFP-lineage fibroblast scRNAseq tSNE plot Fibrotic markers TGFB1, ITGAM, ITGB2, ADAM8, SDC3, CCL3/CCL4/CCL6/CCL9, CD14/CD74/IL1b

FIGS. 24A-24B are UMAP graphs of PBS+Veh.Srb+BLR200 (8 integrated samples).

FIGS. 25A-25K are UMAP graphs of the Cluster Identity detected by established markers, Reticular markers Nexn (FIG. 25A), Trim63 (FIG. 25B), Actn2 (FIG. 25C), Hspb7 (FIG. 25D); Papillary markers: Crabp1 (FIG. 25E), Defb8 (FIG. 25F); Universal markers C3 (FIG. 25G), Pi16 (FIG. 25KH), Col15ai (FIG. 25I); and Epithelial markers: Krt16 (FIG. 25J), Cdh1 (FIG. 25K), Tfap2c (FIG. 25L)

FIG. 26 is a graph showing the Expansion of epithelial and reticular cell populations that are blocked by BLR200 (8 samples).

TABLE 3
Spatial transcriptomics: Signaling Pathways Sensitive to BLR200 (8 samples)

| | Fold | |
|---|---|---|
| | Enrichment | Genes |
| Focal Adhesion | 1.72 | LAMA5, MYLK2, SHC1, LAMA4, PTEN, PIK3R2, |
| | | PIK3R1, MYL12A, MYLK, RAP1B, PPP1CB, |
| | | PAK1, ERBB2, AKT1, PAK6, PIP5K1A, FLNB, |
| | | ITGAV, RAC1, FLNC, PAK2, MAPK3, JUN, |
| | | PPP1R12A, CAV2, ACTN1, CAV1, VEGFB, EMP2, |
| | | PPP1CA, MYLPF, RAPGEF1, TLN1, RAF1, |
| | | PPP1R12B, PPP1R12C |
| WNT Signaling | 1.58 | CTBP1, LRP5, CUL1, PRICKLE3, LRP6, PPP3CA, |
| Pathway | | MYC, DVL3, EP300, RAC1, CAMK2G, PRKACB, |
| | | WNT4, TLE3, WNT10A, JUN, CREBBP, FZD4, |
| | | CSNK1A1, FZD7, CSNK2A2, FZD6, NFATC3, |
| | | RBX1, PLCB3, DAAM1, CSNK2B, PPARD |
| Proteoglycans | 1.99 | PIK3R2, PIK3R1, IQGAP1, SLC9A1, PPP1CB, |
| in Cancer | | NRAS, PAK1, ERBB3, MYC, GPC1, ERBB2, |
| | | PLCG2, AKT1, FLNB, ITGAV, RAC1, FLNC, |
| | | CAMK2G, PRKACB, EIF4B, WNT4, MAPK3, |
| | | WNT10A, PPP1R12A, FZD4, CAV2, FZD7, CAV1, |
| | | FZD6, STAT3, GAB1, ANK3, NUDT16L1, |
| | | MAPK14, ANK1, PPP1CA, SDC1, ARHGEF1, |
| | | RAF1, EZR, PPP1R12B, PPP1R12C |
| Oxidative | 2.59 | NDUFB9, NDUFB8, NDUFA13, NDUFA11, |
| Phosphorylation | | NDUFB10, NDUFB5, NDUFA10, COX7A2, |
| | | UQCR10, COX7A1, COX6A2, UQCRH, UQCRFS1, |
| | | CYC1, NDUFV2, NDUFV1, ATP6V1D, |
| | | ATP6V1C1, ATP6V1C2, NDUFA9, NDUFA8, |
| | | ATP6V0B, NDUFA7, COX8B, NDUFA6, SDHC, |
| | | SDHD, COX6C, SDHA, COX7A2L, NDUFS8, |
| | | NDUFS7, UQCRC1, NDUFS2, NDUFS1, |
| | | ATP6V0D1 |
| Cellular | 1.90 | RB1, H2-T22, H2-Q6, PTEN, CALML3, PIK3R2, |
| Senescence | | PIK3R1, FOXO3, FOXO1, PPP1CB, PPP3CA, |
| | | NRAS, MYC, EIF4EBP1, AKT1, E2F4, MAPK3, |
| | | MAP2K3, GADD45B, NFATC3, HIPK1, MAPK14, |
| | | HIPK3, GADD45G, HIPK2, PPP1CA, RBL2, CDK4, |
| | | RHEB, MAPKAPK2, VDAC2, CALM4, RAF1, |
| | | CALM1, SLC25A5, SLC25A4 |
| HIF-1 Signaling | 2.13 | PIK3R2, PIK3R1, ENO3, HK2, HK1, MKNK2, |
| Pathway | | ERBB2, PLCG2, PGK1, EIF4EBP1, AKT1, EP300, |
| | | CAMK2G, MAPK3, EGLN1, EGLN3, CREBBP, |
| | | PDHA1, IFNGR2, STAT3, RBX1, ALDOA, EIF4E2, |
| | | PFKM, PFKP |
| FoxO Signaling | 1.78 | USP7, CREBBP, GADD45B, STAT3, PTEN, |
| Pathway | | PIK3R2, FOXO4, SLC2A4, PIK3R1, FOXO3, |
| | | MAPK14, FBXO32, FOXO1, GADD45G, RBL2, |
| | | IKBKB, NRAS, STK11, CAT, AKT1, EP300, SGK1, |
| | | RAF1, MAPK3 |
| Regulation of | 1.68 | CYFIP1, MYLK2, ARPC5L, BRK1, PIK3R2, |
| Actin | | PIK3R1, IQGAP1, MYL12A, SLC9A1, MYLK, |
| Cytoskeleton | | GNA13, PPP1CB, NRAS, PAK1, CFL2, AKT1, |
| | | PAK6, PIP5K1A, ITGAV, RAC1, PIP4K2C, PAK2, |
| | | WASF2, MAPK3, PPPIR12A, ACTN1, LIMK2, |
| | | BAIAP2, GNG12, PPP1CA, MYLPF, RGCC, |
| | | ARPC2, ARHGEF1, RAF1, EZR, PPP1R12B, |
| | | ARHGEF7, PPP1R12C, FGFR3 |
| Notch Signaling | 2.31 | PSENEN, TLE3, HDAC2, CREBBP, JAG1, |
| Pathway | | NOTCH1, CTBP1, NRARP, DTX2, NCOR2, |
| | | ATXN1, SNW1, NUMB, DVL3, EP300 |
| Hippo Signaling | 1.54 | YAP1, PARD6G, LIMD1, PPP1CB, PPP2CA, |
| Signaling | | PPP2CB, CDH1, MYC, DVL3, CCN2, WNT4, |
| | | YWHAH, WNT10A, FZD4, FZD7, FZD6, YWHAZ, |
| | | PPP1CA, LATS1, MOB1B, MOB1A, DLG1, ID1, |
| | | SNAI2, BMPR1A |

FIG. 27A is a graph showing the Pharmanest digital analysis of trichrome (study of the structure of collagen in the animal model.

FIG. 27B is a graph showing the Fib Morphometric Composite Score—ASBLD. Collagen structure tends to be more uniform in scrambled; reversed in BLR-200 (kurtosity).

FIG. 27C is a graph showing the qFT Trajectory (Collagen Content).

FIG. 27D is a graph showing the Assembled collagen in PBS, Bleomycin+scrambled, and Bleomycin+BLR-200.

### Example: Intra-tracheal Bleomycin Study to Determine the Effect of BLR-200 on Lung Fibrosis

Intra-tracheal bleomycin injections on Animals—C57 BL/6 J mice (Male), Age—6 weeks were conducted. BLR 200 and Scrambled, negative control peptide injections (intraperitoneal) were conducted on day 1 through day 20 in three- or two-day intervals. On day 22, Blood, BAL fluid and Lung collection and were Trichrome stained. FIGS. 28A-28C show the Effect of BLR-200 on Tissue Histology, PBS (FIG. 28A), Bleomycin+Scrambled (FIG. 28B), Bleomycin+BLR 200 (FIG. 28C), showing C: Collagen deposition. FIGS. 28D-28F show the Effect of BLR-200 on Tissue H&E staining in PBS (FIG. 28D), Bleomycin+Scrambled (FIG. 28E) and Bleomycin+BLR200 (FIG. 28F) showing A: Air spaces of alveolus, B: Respiratory Bronchiole, BV: Blood vessel. These FIGS. 28A-28F show that in the lung disease that not all of the same genes and proteins that are associated with fibrosis that are increased in the skin disease are also increased in the lung.

FIGS. 29A-29D are graphs showing the Effects of BLR-200 Treatment on Biomarkers of IPF/ILD, Collagen Lung (FIG. 29A, hydroxyproline levels), Plasma TGF-B1 levels (FIG. 29B), plasma MMP 7 levels (FIG. 29C), and BAL fluid MMP 7 levels (FIG. 29D, Bronchoalveolar Lavage).

FIGS. 30A-30J are graphs showing the lung Tissue Changes in IPF Biomarkers by qPCR (mRNA Levels): Effect of BLR200. Relative expression of TNC, PAI-1, IL-33, CCN-1, CCN4, FN1, THBS1, ACAT2, IL 1 beta, TNF-alpha, COL3A1, CXCL2 (FIGS. 30A-30J). FIGS. 31A-31F are graphs showing the Gene Expression Unaltered by Disease or by BLR-200.

### PUBLICATIONS

Abd El Kader, T., Kubota, S., Janune, D., Nishida, T., Hattori, T., Aoyama, E., Perbal, B., Kuboki, T., & Takigawa, M. (2012). Anti-fibrotic effect of CCN3 accompanied by altered gene expression profile of the CCN family. Journal of Cell Communication and Signaling, 7(1), 11-18. https://doi.org/10.1007/S12079-012-0180-4
Allanore, Y., Simms, R., Distler, O., Trojanowska, M., Pope, J., Denton, C. P., & Varga, J. (2015). Systemic sclerosis. Nature Reviews Disease Primers, 1(15002), 1-21. https://doi.org/10.1038/nrdp.2015.2
Andenos, K., Lunde, I. G., Mohammadzadeh, N., Dahl, C. P., Aronsen, J. M., Strand, M. E., Palmero, S., Sjaastad, I., Christensen, G., Engebretsen, K. V. T., & Tonnessen, T. (2018). The extracellular matrix proteoglycan fibromodulin is upregulated in clinical and experimental heart failure and affects cardiac remodeling. PLoS ONE, 13(7), e0201422. https://doi.org/10.1371/JOURNAL.PONE.0201422
Armstrong, J. J. (2019). Modulation of Inflammation Driven Wound Healing after Glaucoma Surgery. Electronic Thesis and Dissertation Repository, 6534.
Belloli, L., Carlo-Stella, N., Ciocia, G., Chiti, A., Massarotti, M., & Marasini, B. (2008). Myocardial involvement in systemic sclerosis. Rheumatology. https://doi.org/10.1093/rheumatology/ken186
Brenner, M. C., Krzyzanski, W., Chou, J. Z., Signore, P. E., Fung, C. K., Guzman, D., Li, D., Zhang, W., Olsen, D. R., Nguyen, V.-T. L., Koo, C. W., Sternlicht, M. D., & Lipson, K. E. (2016). FG-3019, a Human Monoclonal Antibody Recognizing Connective Tissue Growth Factor, is Subject to Target-Mediated Drug Disposition. Pharmaceutical Research, 33(8), 1849. https://doi.org/10.1007/511095-016-1918-0
Chadli, L., Sotthewes, B., Li, K., Andersen, S. N., Cahir-McFarland, E., Cheung, M., Cullen, P., Dorjée, A., de Vries-Bouwstra, J. K., Huizinga, T. W. J., Fischer, D. F., DeGroot, J., Viney, J. L., Zheng, T. S., Aarbiou, J., & Gardet, A. (2019). Identification of regulators of the myofibroblast phenotype of primary dermal fibroblasts from early diffuse systemic sclerosis patients. Scientific Reports, 9(1), 1-12. https://doi.org/10.1038/s41598-019-41153-w
Chen, C. C., Chen, N., & Lau, L. F. (2001). The Angiogenic Factors Cyr61 and Connective Tissue Growth Factor Induce Adhesive Signaling in Primary Human Skin Fibroblasts. Journal of Biological Chemistry. https://doi.org/10.1074/jbc.M008087200
Chen, C. C., & Lau, L. F. (2009). Functions and mechanisms of action of CCN matricellular proteins. International Journal of Biochemistry and Cell Biology. https://doi.org/10.1016/j.biocel.2008.07.025
Chen, L., Li, S., & Li, W. (2018). LOX/LOXL in pulmonary fibrosis: potential therapeutic targets. Journal of Drug Targeting, 27(7), 790-796. https://doi.org/10.1080/1061186X.2018.1550649
Czirják, L., Foeldvari, I., & Müller-Ladner, U. (2008). Skin involvement in systemic sclerosis. Rheumatology. https://doi.org/10.1093/rheumatology/ken309
Darby, I., Skalli, O., & Gabbiani, G. (1990). Alpha-smooth muscle actin is transiently expressed by myofibroblasts during experimental wound healing. Laboratory Investigation; a Journal of Technical Methods and Pathology, 63(1), 21-29. https://europepmc.org/article/med/2197503
di Carlo, S. E., & Peduto, L. (2018). The perivascular origin of pathological fibroblasts. Journal of Clinical Investigation, 128(1), 54-63. https://doi.org/10.1172/JCI93558
Domogatskaya, A., Rodin, S., & Tryggvason, K. (2012). Functional diversity of laminins. Annual Review of Cell and Developmental Biology, 28, 523-553. https://doi.org/10.1146/ANNUREV-CELLBIO-101011-155750
Dorn, L. E., Petrosino, J. M., Wright, P., & Accornero, F. (2018). CTGF/CCN2 is an autocrine regulator of cardiac fibrosis. Journal of Molecular and Cellular Cardiology, 121, 205-211. https://doi.org/10.1016/J.YJMCC.2018.07.130
Dupont, S., Morsut, L., Aragona, M., Enzo, E., Giulitti, S., Cordenonsi, M., Zanconato, F., le Digabel, J., Forcato, M., Bicciato, S., Elvassore, N., & Piccolo, S. (2011). Role of YAP/TAZ in mechanotransduction. Nature, 474(7350), 179-183. https://doi.org/10.1038/nature10137
Dziadzio, M., Usinger, W., Leask, A., Abraham, D., Black, C. M., Denton, C., & Stratton, R. (2005). N-terminal connective tissue growth factor is a marker of the fibrotic phenotype in scleroderma. Monthly Journal of the Association of Physicians. https://doi.org/10.1093/qjmed/hci078
Gabbiani, G. (2003). The myofibroblast in wound healing and fibrocontractive diseases. In Journal of Pathology. https://doi.org/10.1002/path.1427
Gardner, H., Shearstone, J. R., Bandaru, R., Crowell, T., Lynes, M., Trojanowska, M., Pannu, J., Smith, E., Jablonska, S., Blaszczyk, M., Tan, F. K., & Mayes, M. D. (2006). Gene profiling of scleroderma skin reveals robust signatures of disease that are imperfectly reflected in the transcript profiles of explanted fibroblasts. Arthritis & Rheumatology, 54(6), 1961-1973. https://doi.org/10.1002/ART.21894
Gjaltema, R. A. F., de Rond, S., Rots, M. G., & Bank, R. A. (2015). Procollagen Lysyl Hydroxylase 2 Expression Is Regulated by an Alternative Downstream Transforming Growth Factor-1 Activation Mechanism *. Journal of Biological Chemistry, 290(47), 28465-28476. https://doi.org/10.1074/jbc.M114.634311
Hinz, B. (2010). The myofibroblast: Paradigm for a mechanically active cell. Journal of Biomechanics. https://doi.org/10.1016/j.jbiomech.2009.09.020
Hinz, B. (2015). Myofibroblasts. In Experimental Eye Research. https://doi.org/10.1016/j.exer.2015.07.009
Hoberg, M., Rudert, M., Pap, T., Klein, G., Gay, S., & Aicher, W. K. (2007). Attachment to laminin-111 facilitates transforming growth factor β-induced expression of matrix metalloproteinase-3 in synovial fibroblasts. Annals of the Rheumatic Diseases, 66(4), 451. https://doi.org/10.1136/ARD.2006.060228
Holmes, A. M., Ponticos, M., Shi-wen, X., Denton, C. P., & Abraham, D. J. (2011). Elevated CCN2 expression in scleroderma: a putative role for the TGFβ accessory receptors TGFβRIII and endoglin. Journal of Cell Communication and Signaling, 5(3), 177. https://doi.org/10.1007/S12079-011-0140-4
Hung, C., Linn, G., Chow, Y.-H., Kobayashi, A., Mittelsteadt, K., Altemeier, W. A., Gharib, S. A., Schnapp, L. M., & Duffield, J. S. (2013). Role of Lung Pericytes and Resident Fibroblasts in the Pathogenesis of Pulmonary Fibrosis. American Journal of Respiratory and Critical Care Medicine, 188(7), 820-830. https://doi.org/10.1164/RCCM.201212-2297OC
Jun, J.-I., & Lau, L. F. (2011). Taking aim at the extracellular matrix: CCN proteins as emerging therapeutic targets. Nature Reviews Drug Discovery, 10(12), 945-963. https://doi.org/10.1038/nrd3599
Kalamajski, S., Bihan, D., Bonna, A., Rubin, K., & Farndale, R. W. (2016). Fibromodulin Interacts with Collagen Cross-linking Sites and Activates Lysyl Oxidase. Journal of Biological Chemistry, 291(15), 7960. https://doi.org/10.1074/JBC.M115.693408
Kanaizuka, I., Sasaki, T., & Nakajima, H. (1991). Type IV collagen and laminin levels in the sera from patients with systemic scleroderma (PSS). Japanese Journal of Dermatology, 101(8), 819-823.
King, J., Abraham, D., & Stratton, R. (2018). Chemokines in systemic sclerosis. Immunology Letters, 195, 68-75. https://doi.org/10.1016/J.IMLET.2017.12.001
Kubota, S., & Takigawa, M. (2007). CCN family proteins and angiogenesis: from embryo to adulthood. Angiogenesis, 10(1), 1-11. https://doi.org/10.1007/S10456-006-9058-5
Leask, A. (2013). CCN2: a novel, specific and valid target for anti-fibrotic drug intervention. Expert Opinion on Therapeutic Targets, 17(9), 1067-1071. https://doi.org/10.1517/14728222.2013.812074
Leask, A. (2015). Matrix remodeling in systemic sclerosis. In Seminars in Immunopathology. https://doi.org/10.1007/s00281-015-0508-2
Leask, A., & Abraham, D. (2004). TGF-β signaling and the fibrotic response. FASEB Journal, 18(7), 816-827. https://doi.org/10.1096/fj.03-1273rev
Leask, A., & Abraham, D. J. (2006). All in the CCN family: essential matricellular signaling modulators emerge from the bunker. Journal of Cell Science. https://doi.org/10.1242/jcs.03270
Leask, A., Holmes, A., Black, C. M., & Abraham, D. J. (2003). Connective tissue growth factor gene regulation: Requirements for its induction by transforming growth factor-02 in fibroblasts. Journal of Biological Chemistry. https://doi.org/10.1074/jbc.M210366200
Lemaire, R., Farina, G., Bayle, J., Dimarzio, M., Pendergrass, S. A., Milano, A., Whitfield, M. L., & Lafyatis, R. (2010). Antagonistic Effect of the Matricellular Signaling Protein CCN3 on TGF-β- and Wnt-Mediated Fibrillinogenesis in Systemic Sclerosis and Marfan Syndrome. Journal of Investigative Dermatology, 130(6), 1514-1523. https://doi.org/10.1038/JID.2010.15
Liu, P., Chen, H., Yan, L., & Sun, Y. (2020). Laminin a5 modulates fibroblast proliferation in epidural fibrosis through the PI3K/AKT/mTOR signaling pathway. Molecular Medicine Reports, 21(3), 1500. https://doi.org/10.3892/MMR.2020.10967
Liu, S., Herault, Y., Pavlovic, G., & Leask, A. (2014). Skin progenitor cells contribute to bleomycin-induced skin fibrosis. Arthritis & Rheumatology. https://doi.org/10.1002/art.38276
Liu, S., Shi-Wen, X., Abraham, D. J., & Leask, A. (2011). CCN2 is required for bleomycin-induced skin fibrosis in mice. Arthritis & Rheumatology, 63(1), 239-246. https://doi.org/10.1002/art.30074
Lóránd, V., Czirják, L., & Minier, T. (2014). Musculoskeletal involvement in systemic sclerosis. La Presse Médicale, 43(10), 315-328. https://doi.org/10.1016/j.lpm.2014.03.027
Makino, K., Makino, T., Stawski, L., Lipson, K. E., Leask, A., & Trojanowska, M. (2017). Anti-connective tissue growth factor (CTGF/CCN2) monoclonal antibody attenuates skin fibrosis in mice models of systemic sclerosis. Arthritis Research & Therapy, 19(1), 134. https://doi.org/10.1186/s13075-017-1356-3
Meyringer, R., Neumann, E., Judex, M., Landthaler, M., Kullmann, F., Scholmerich, J., Gay, S., Tarner, I. H., Distler, O., & Müller-Ladner, U. (2007). Analysis of gene expression patterns in systemic sclerosis fibroblasts using RNA arbitrarily primed-polymerase chain reaction for differential display. Journal of Rheumatology, 34(4), 747-753.
Mormone, E., Lu, Y., Ge, X., Fiel, M. I., & Nieto, N. (2012). Fibromodulin, an Oxidative Stress-Sensitive Proteoglycan, Regulates the Fibrogenic Response to Liver Injury in Mice. Gastroenterology, 142(3), 621. https://doi.org/10.1053/J.GASTRO.2011.11.029
Nguyen, X.-X., Nishimoto, T., Takihara, T., Mlakar, L., Bradshaw, A. D., & Feghali-Bostwick, C. (2021). Lysyl oxidase directly contributes to extracellular matrix production and fibrosis in systemic sclerosis. Lung Cellular and Molecular Physiology, 320(1), L29-L40. https://doi.org/10.1152/AJPLUNG.00173.2020
Parapuram, S. K., Thompson, K., Tsang, M., Hutchenreuther, J., Bekking, C., Liu, S., & Leask, A. (2015). Loss of PTEN expression by mouse fibroblasts results in lung fibrosis through a CCN2-dependent mechanism. Matrix Biology. https://doi.org/10.1016/j.matbio.2015.01.017
Perbal, B. (2004). CCN proteins: Multifunctional signalling regulators. In Lancet. https://doi.org/10.1016/S0140-6736(03)15172-0
Perbal, B. (2013). CCN proteins: A centralized communication network. Journal of Cell Communication and Signaling. https://doi.org/10.1007/s12079-013-0193-7
Perbal, B. (2018). The concept of the CCN protein family revisited: a centralized coordination network. Journal of Cell Communication and Signaling, 12(1), 3. https://doi.org/10.1007/S12079-018-0455-5
Petz, M., Them, N., Huber, H., Beug, H., & Mikulits, W. (2012). La enhances IRES-mediated translation of laminin B1 during malignant epithelial to mesenchymal transition. Nucleic Acids Research, 40(1), 302. https://doi.org/10.1093/NAR/GKR717
Phanish, M. K., Winn, S. K., & Dockrell, M. E. C. (2010). Connective Tissue Growth Factor-(CTGF, CCN2)—A Marker, Mediator and Therapeutic Target for Renal Fibrosis. Nephron Experimental Nephrology, 114(3), e83-e92. https://doi.org/10.1159/000262316
Quesnel, K., Shi-wen, X., Hutchenreuther, J., Xiao, Y., Liu, S., Peidl, A., Naskar, D., Siqueira, W. L., O'Gorman, D. B., Hinz, B., Stratton, R. J., & Leask, A. (2019). CCN1 expression by fibroblasts is required for bleomycin-induced skin fibrosis. Matrix Biology Plus, 3, 100009. https://doi.org/10.1016/J.MBPLUS.2019.100009
Rachfal, A. W., & Brigstock, D. R. (2003). Connective tissue growth factor (CTGF/CCN2) in hepatic fibrosis. Hepatology Research, 26(1), 1-9. https://doi.org/10.1016/S1386-6346(03)00115-3
Rajkumar, V. S., Howell, K., Csiszar, K., Denton, C. P., Black, C. M., & Abraham, D. J. (2005). Shared expression of phenotypic markers in systemic sclerosis indicates a convergence of pericytes and fibroblasts to a myofibroblast lineage in fibrosis. Arthritis Research & Therapy, 7(5), R1123. https://doi.org/10.1186/AR1790
Rigau, A. R., Luber, M., & Distler, J. H. (2021). Mouse Models of Skin Fibrosis. Methods in Molecular Biology, 2299, 371-383. https://doi.org/10.1007/978-1-0716-1382-5_25
Riser, B. L., Najmabadi, F., Garchow, K., Barnes, J. L., Peterson, D. R., & Sukowski, E. J. (2014). Treatment with the Matricellular Protein CCN3 Blocks and/or Reverses Fibrosis Development in Obesity with Diabetic Nephropathy. American Journal of Pathology, 184(11), 2908-2921. https://doi.org/10.1016/j.ajpath.2014.07.009
Riser, B. L., Najmabadi, F., Perbal, B., Peterson, D. R., Rambow, J. A., Riser, M. L., Sukowski, E., Yeger, H., & Riser, S. C. (2009). CCN3 (NOV) is a negative regulator of CCN2 (CTGF) and a novel endogenous inhibitor of the fibrotic pathway in an in vitro model of renal disease. American Journal of Pathology, 174(5), 1725-1734. https://doi.org/10.2353/ajpath.2009.080241
Riser, B. L., Najmabadi, F., Perbal, B., Rambow, J. A., Riser, M. L., Sukowski, E., Yeger, H., Riser, S. C., & Peterson, D. R. (2010). CCN3/CCN2 regulation and the fibrosis of diabetic renal disease. Journal of Cell Communication and Signaling, 4(1), 39. https://doi.org/10.1007/s12079-010-0085-z
Robinson, M. D., McCarthy, D. J., Smyth, G. K. (2010). edgeR: a Bioconductor package for differential expression analysis of digital gene expression data. Bioinformatics, 26(1), 139-140. https://doi.org/10.1093/bioinformatics/btp616
Santos, V. N. dos, Leite-Mór, M. M. B., Kondo, M., Martins, J. R., Nader, H., Lanzoni, V. P., & Parise, E. R. (2005). Serum laminin, type IV collagen and hyaluronan as fibrosis markers in non-alcoholic fatty liver disease. Brazilian Journal of Medical and Biological Research, 38(5), 747-753. https://doi.org/10.1590/S0100-879X2005000500012
Schulz, J. N., Plomann, M., Sengle, G., Gullberg, D., Krieg, T., & Eckes, B. (2018). New developments on skin fibrosis—Essential signals emanating from the extracellular matrix for the control of myofibroblasts. Matrix Biology, 68-69, 522-532. https://doi.org/10.1016/J.MATBIO.2018.01.025
Shi-wen, X., Racanelli, M., Ali, A., Simon, A., Quesnel, K., Stratton, R. J., & Leask, A. (2021). Verteporfin inhibits the persistent fibrotic phenotype of lesional scleroderma dermal fibroblasts. Journal of Cell Communication and Signaling, 15(1), 71. https://doi.org/10.1007/S12079-020-00596-X
Sonnylal, S., Shi-Wen, X., Leoni, P., Naff, K., van Pelt, C. S., Nakamura, H., Leask, A., Abraham, D., Bou-Gharios, G., & de Crombrugghe, B. (2010). Selective Expression of Connective Tissue Growth Factor in Fibroblasts In Vivo Promotes Systemic Tissue Fibrosis. Arthritis & Rheumatology, 62(5), 1-17. https://doi.org/10.1002/art.27382
Sun, Y., Wang, T. L., Toh, W. S., & Pei, M. (2017). The role of laminins in cartilaginous tissues: from development to regeneration. European Cells & Materials, 34, 54. https://doi.org/10.22203/ECM.V034A03
Talior-Volodarsky, I., Connelly, K. A., Arora, P. D., Gullberg, D., & McCulloch, C. A. (2012). α11 integrin stimulates myofibroblast differentiation in diabetic cardiomyopathy. Cardiovascular Research, 96(2), 265-275. https://doi.org/10.1093/CVR/CVS259
Thompson, K., Murphy-Marshman, H., & Leask, A. (2014). ALK5 inhibition blocks TGFβ-induced CCN1 expression in human foreskin fibroblasts. Journal of Cell Communication and Signaling, 8(1), 59. https://doi.org/10.1007/s12079-014-0229-7
Toyama, T., Looney, A. P., Baker, B. M., Stawski, L., Haines, P., Simms, R., Szymaniak, A. D., Varelas, X., & Trojanowska, M. (2018). Therapeutic targeting of TAZ and YAP by dimethyl fumarate in systemic sclerosis fibrosis. Journal of Investigative Dermatology, 138(1), 88. https://doi.org/10.1016/J.JID.2017.08.024
Tsang, M., & Leask, A. (2014). CCN2 is required for recruitment of Sox2-expressing cells during cutaneous tissue repair. Journal of Cell Communication and Signaling, 9(4), 341-346. https://doi.org/10.1007/S12079-014-0245-7
Tsang, M., Quesnel, K., Vincent, K., Hutchenreuther, J., Postovit, L.-M., & Leask, A. (2019). Insights into Fibroblast Plasticity: Cellular Communication Network 2 Is Required for Activation of Cancer-Associated Fibroblasts in a Murine Model of Melanoma. American Journal of Pathology, 190(1), 206-221. https://doi.org/10.1016/J.AJPATH.2019.09.006
van der Slot, A. J., Zuurmond, A.-M., Bardoel, A. F. J., Wijmenga, C., Pruijs, H. E. H., Sillence, D. O., Brinckmann, J., Abraham, D. J., Black, C. M., Verzijl, N., DeGroot, J., Hanemaaijer, R., TeKoppele, J. M., Huizinga, T. W. J., & Bank, R. A. (2003). Identification of PLOD2 as Telopeptide Lysyl Hydroxylase, an Important Enzyme in Fibrosis *. Journal of Biological Chemistry, 278(42), 40967-40972. https://doi.org/10.1074/JBC.M307380200
Varga, J., & Abraham, D. (2007). Systemic sclerosis: a prototypic multisystem fibrotic disorder. Journal of Clinical Investigation, 117(3), 557-567. https://doi.org/10.1172/JCI31139
Woodfin, A., Voisin, M.-B., & Nourshargh, S. (2007). PECAM-1: A Multi-Functional Molecule in Inflammation and Vascular Biology. Arteriosclerosis, Thrombosis, and Vascular Biology, 27(12), 2514-2523. https://doi.org/10.1161/ATVBAHA.107.151456
Yamada, M., Hirabayashi, K., Kawanishi, A., Hadano, A., Takanashi, Y., Izumi, H., Kawaguchi, Y., Mine, T., Nakamura, N., & Nakagohri, T. (2017). Nectin-1 expression in cancer-associated fibroblasts is a predictor of poor prognosis for pancreatic ductal adenocarcinoma. Surgery Today, 48(5), 510-516. https://doi.org/10.1007/S00595-017-1618-3
Yamamoto, T. (2006). The bleomycin-induced scleroderma model: what have we learned for scleroderma pathogenesis? Archives of Dermatological Research, 297(8), 333-344. https://doi.org/10.1007/500403-005-0635-Z
Yamamoto, T., & Katayama, I. (2011). Vascular Changes in Bleomycin-Induced Scleroderma. International Journal of Rheumatology, 2011, 1-5. https://doi.org/10.1155/2011/270938
Yamamoto, T., Takagawa, S., Katayama, I., Yamazaki, K., Hamazaki, Y., Shinkai, H., & Nishioka, K. (1999). Animal model of sclerotic skin. I: Local injections of bleomycin induce sclerotic skin mimicking scleroderma. Journal of Investigative Dermatology. https://doi.org/10.1046/j.1523-1747.1999.00528.x
Zeltz, C., Alam, J., Liu, H., Erusappan, P. M., Hoschuetzky, H., Molven, A., Parajuli, H., Cukierman, E., Costea, D. E., Lu, N., & Gullberg, D. (2019). α11β1 integrin is induced in a subset of cancer-associated fibroblasts in desmoplastic tumor stroma and mediates in vitro cell migration. Cancers, 11(6), 765. https://doi.org/10.3390/CANCERS11060765

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of diagnosing a disease state or condition comprising:
using a gene expression pattern or a protein expression pattern in a fibroblast cell, a progenitor cell, a stem cell, a myofibroblast cell, or a specific myofibroblast population with specific markers to determine early onset of a diseased state, a progression of the disease state, or a regression of the disease state in response to a therapeutic treatment;
a. whereby the disease state is cancer, fibrosis, autoimmune, scleroderma, or systemic fibrosis.

2. The method of claim 1, wherein the gene expression or protein expression pattern is in cxcl13, Csf2, Il33, IL6, Cxcl2 and the inflammasome NLRP-3 including IL-18, tLr-2, Il1r1, Il1r2, wnt10b or Edn1.

3. A method of screening a drug or a therapeutic for a disease, comprising:
using a gene expression pattern or a protein expression pattern in a fibroblast cell, a progenitor cell, a stem cell, a myofibroblast cell, or a specific defined population of myofibroblasts carrying specific markers;
examining the drug or the therapeutic on the protein expression changes and the gene expression changes over a period of time; and
selecting the drug or therapeutic to treat the disease based upon a threshold change in the protein expression or gene expression.

4. The method of claim 3, wherein the drug or therapeutic suppresses myofibroblast differentiation.

5. The method of claim 3, wherein the drug or therapeutic reverses excessive collagen deposition.

6. The method of claim 3, wherein the drug or therapeutic prevents the gene or protein expression increase in Plod2 and Lox.

7. The method of claim 3, wherein the drug or therapeutic decreases the number of αSMA-expressing myofibroblasts.

8. The method of claim 3, wherein the drug or therapeutic prevents activation of highly contractile myofibroblasts in the fibrotic lesion.

9. The method of claim 3, wherein the drug or therapeutic prevents protein expression of extracellular matrix organization (R-MMU-1474244), assembly of collagen fibrils and other multimeric structures (nectin-2, pecam-1, fibromodulin), laminin interactions (a3, a5, b3, c2) and collagen formation (R-MMU-1474290), ECM-associated proteins, including laminin (lam)a5, lama3, lamb3, lamc2, nectin-2, pecam-1, and fibromodulin.

10. The method of claim 3, wherein the drug or therapeutic decreases the protein expression and gene expression of either or all in combination CCN1, CCN2, CCN3, and CCN4.

11. The method of claim 3, wherein the drug or therapeutic prevents Sox2 and Itga11 expression.

12. The method of claim 3, wherein the drug or therapeutic biologically alters YAP1-expression.

13. The method of claim 3, wherein the drug or therapeutic decreases expression of cadherin 11 (CDH11), Smad3, tenascin-C (TNC), YAP1, Sox2, WNT4, frizzled 6 (FZD6) and PLOD2.

14. The method of claim 3, wherein the drug or therapeutic decreases the expression of keratinocyte activation, myofibroblast contraction, keratin 16, keratin 6, keratin 5, keratin 14, keratin 1, fillagrin, and hornerin, Myosin light chain 6b and tropomyosin 3, Fibrillin-1, and dermatopontin.

15. The method of claim 3, wherein the drug or therapeutic prevents the increase in IL6 and Cxcl2 expression in the inflammatory fibroblast subpopulation.

16. The method of claim 3, wherein the drug or therapeutic impairs the expression of interleukin-18 (Il8), toll-like receptor 2 (Tlr2), interleukin-1 receptor type 1 (Il1r1), and interleukin-1 receptor type 2 (Il1r2).

17. The method of claim 3, wherein the drug or therapeutic prevents the increase in the pro-fibrotic transcription factor Egr1 expression.

18. The method of claim 3, wherein the drug or therapeutic prevents the expression of mesenchymal markers Tek, Osr2, and Sfrp2.

19. The method of claim 3, wherein the disease is a fibrotic disease, scleroderma and systemic sclerosis, interstitial lung fibrosis, idiopathic pulmonary fibrosis.

20. A method of determining the stage of a disease, comprising: delivering a therapeutic effective amount of BLR-200 to regulate the expression of a biological pathway to the disease; and predetermining a drug response in the disease where the biological pathways are selected from the group of: NLRP3, inflammasome, and the like.


SEQUENCE LISTING (20 sequences)

SEQ ID NO: 1 - DNA, 19 bp, Mus musculus
  source 1..19: mol_type = genomic DNA
gtaacccgttgaacccatt

SEQ ID NO: 2 - DNA, 20 bp, Mus musculus
  source 1..20: mol_type = genomic DNA
ccatccaatcggtagtagcg

SEQ ID NO: 3 - DNA, 18 bp, Mus musculus
  source 1..18: mol_type = genomic DNA
catccgacactgctgaca

SEQ ID NO: 4 - DNA, 24 bp, Mus musculus
  source 1..24: mol_type = genomic DNA
aggtctcaaacataatctgggtca

SEQ ID NO: 5 - DNA, 22 bp, Mus musculus
  source 1..22: mol_type = genomic DNA
tctgcgctaaacaactcaacga

SEQ ID NO: 6 - DNA, 20 bp, Mus musculus
  source 1..20: mol_type = genomic DNA
gcagatccctttcagagcgg

SEQ ID NO: 7 - DNA, 20 bp, Mus musculus
  source 1..20: mol_type = genomic DNA
tgctgtgcatcctcctaccg

SEQ ID NO: 8 - DNA, 20 bp, Mus musculus
  source 1..20: mol_type = genomic DNA
cagagagcgaggagcaccaa

SEQ ID NO: 9 - DNA, 20 bp, Mus musculus
  source 1..20: mol_type = genomic DNA
gcggggagagttgttctgag

SEQ ID NO: 10 - DNA, 20 bp, Mus musculus
  source 1..20: mol_type = genomic DNA
gtctccctctggaaccatgc

SEQ ID NO: 11 - DNA, 20 bp, Mus musculus
  source 1..20: mol_type = genomic DNA
cgctgtgaatcagacgaggt

SEQ ID NO: 12 - DNA, 19 bp, Mus musculus
  source 1..19: mol_type = genomic DNA
cccacagggctattcttgt

SEQ ID NO: 13 - DNA, 19 bp, Mus musculus
  source 1..19: mol_type = genomic DNA
ctccgcagcgaaacgacag

SEQ ID NO: 14 - DNA, 20 bp, Mus musculus
  source 1..20: mol_type = genomic DNA
agtcggcatcacggtttttg

SEQ ID NO: 15 - DNA, 20 bp, Mus musculus
  source 1..20: mol_type = genomic DNA
gaactcggcttcaggtcctc

SEQ ID NO: 16 - DNA, 20 bp, Mus musculus
  source 1..20: mol_type = genomic DNA
agggtcaagccttgggtcta

SEQ ID NO: 17 - DNA, 22 bp, Mus musculus
  source 1..22: mol_type = genomic DNA
caggaacatgggcatggatttc

SEQ ID NO: 18 - DNA, 21 bp, Mus musculus
  source 1..21: mol_type = genomic DNA
gacgtgtcacaagaggagcaa

SEQ ID NO: 19 - DNA, 22 bp, Mus musculus
  source 1..22: mol_type = genomic DNA
aaatgggtatccaacaaatggc

SEQ ID NO: 20 - DNA, 21 bp, Mus musculus
  source 1..21: mol_type = genomic DNA
gtgcgttagaggacaacagga